(19) United States
(12) Patent Application Publication
(10) Pub. No.: US 2024/0424002 A1
(43) Pub. Date: Dec. 26, 2024

(54) METHODS AND COMPOSITIONS FOR TREATING MELANOMA

(71) Applicant: THE REGENTS OF THE UNIVERSITY OF CALIFORNIA, Oakland, CA (US)

(72) Inventors: Thomas GRAEBER, Pacific Palisades, CA (US); Kai SONG, Los Angeles, CA (US)

(73) Assignee: THE REGENTS OF THE UNIVERSITY OF CALIFORNIA, Oakland, CA (US)

(21) Appl. No.: 18/570,569

(22) PCT Filed: Jun. 15, 2022
(86) PCT No.: PCT/US2022/072960

§ 371 Date: Dec. 14, 2023

Related U.S. Application Data
(60) Provisional application No. 63/210,918, filed on Jun. 15, 2021.

(51) Int. Cl. A61K 31/635; A61K 31/4184; A61K 31/437; A61K 31/506; A61K 31/517; A61K 31/519; A61K 39/00; A61K 39/395; A61P 35/00; C12Q 1/6851; C12Q 1/6886; G01N 33/574

(52) U.S. Cl. CPC A61K 31/635; A61K 31/4184; A61K 31/437; A61K 31/506; A61K 31/517; A61K 31/519; A61K 39/3955; A61K 39/4611; A61P 35/00; C12Q 1/6851; C12Q 1/6886; G01N 33/57492; C12Q 2600/106; C12Q 2600/158

(57) ABSTRACT

The current methods and compositions provide for a novel therapeutic method for treating patients diagnosed with melanoma, especially those that have become resistant to certain other therapies. Accordingly, certain aspects of the disclosure relate to a method for treating melanoma in a subject, the method comprising administering a composition comprising a ferroptosis-inducing agent to the subject.

This application is claims benefit of priority of U.S. Provisional Application No. 63/210,918, filed Jun. 15, 2021, which is hereby incorporated by reference in its entirety.

The application contains a Sequence Listing in compliance with ST.25 format and is hereby incorporated by reference in its entirety. Said Sequence Listing, created on May 23, 2024 is named UCLAP0140US_ST25.txt and is 6,472 bytes in size.

This invention was made with government support under Grant Number CA168585, awarded by National Institutes of Health. The government has certain rights in the invention.

### BACKGROUND OF THE INVENTION

### 1. Field of the Invention

Embodiments are directed generally to biology and medicine. In certain aspects methods involve treating cancer patients and determining an optimal therapeutic regimen for the cancer patient. In additional embodiments there are therapeutic compositions and the use of such compositions for the treatment of melanoma.

### 2. Background

Melanoma is a highly aggressive type of skin cancer that arises from melanocytes, the pigment producing cells in the body. The discovery that approximately half of melanomas are driven through BRAF_{V600} mutations and advances in tumor immunology have translated to new targeted and immune therapies with impressive response rates and significantly improved survival (Luke et al., 2017). However, for all these treatment modalities there remain patients that do not respond or ultimately relapse. There is a need in the art for therapies to treat melanoma, particularly those that have acquired resistance mechanisms in response to available therapies.

### SUMMARY OF THE DISCLOSURE

The current methods and compositions provide for a novel therapeutic method for treating patients diagnosed with melanoma, including those that have become resistant to certain other therapies. Accordingly, certain aspects of the disclosure relate to a method for treating melanoma in a subject having amplified BRAF gene, the method comprising administering a composition comprising a ferroptosis-inducing agent to the subject. Also provided is a method for treating melanoma in a subject having one or more of increased MITF expression, increased PGC-1α gene expression, increased mitochondrial respiration programs, increased expression of lipid oxidation pathways, insufficient upregulation of ROS detoxification pathways, decreased levels of reduced glutathione (GSH), reduced glutathione to oxidized glutathione (GSH/GSSG) ratios, decreased levels of total glutathione, and increased expression of NCOA4 the method comprising administering a composition comprising a ferroptosis-inducing agent to the subject. Increased mitochondrial repiration programs may comprises an increase in one or more of tricarboxylic acid (TCA) cycle, electron transport chain (ETC), oxidative phosphorylation, and mitochondrial biogenesis. Increased expression of lipid oxidation pathways comprises increased expression or activity of PPARα and ACOX1 genes or proteins. Insufficient upregulation of ROS detoxification pathways is determined by evaluating glutathione synthetase (GSS) and/or glutathione peroxidase 4 (GPX4). Also provided is a method for treating melanoma in a subject after determining the relative level of lipid oxidation pathways compared to the level of ROS detoxification pathways (via single sample gene set enrichment analysis (ssGSEA) in a biological sample from the subject. Further aspects relate to a method for treating melanoma in a subject comprising administering a composition comprising a ferroptosis-inducing agent to the subject; wherein the subject has been evaluated for one or more of peroxisome proliferator-activated receptor-7 coactivator (PGC-1α), PPARGC1A, PPARα, ACOX, GSS, GPX4, GSH, and glutathione.

Further aspects of the disclosure relate to a method for classifying a subject diagnosed with melanoma, the method comprising: a. obtaining a biological sample from the subject; and b. detecting the expression or activity level of one or more of MITF, PGC-1α, TCA cycle, ETC, oxidative phosphorylation, mitochondrial biogenesis, PPARα, ACOX1, GSS, GPX4; and/or the amplification of the BRAF gene in the biological sample from the subject.

Further aspects of the disclosure relate to a method of predicting sensitivity of melanoma cancer cells to ferroptosis inducers in a subject having melanoma, said method comprising: a. obtaining a biological sample from the subject; b. analyzing, evaluating, or measuring one or more biomarkers comprising one or more of MITF, PGC-1α, TCA cycle, ETC, oxidative phosphorylation, mitochondrial biogenesis, PPARα, ACOX1, GSS, GPX4; and/or the amplification of the BRAF gene in the biological sample from the subject; or the relative level of lipid oxidation pathways compared to the level of ROS detoxification pathways (via single sample gene set enrichment analysis (ssGSEA); c. determining that the subject will be sensitive to ferroptosis inducing agents when MITF activity or expression is increased, PGC-1α activity or expression is increased, one or more of TCA cycle, ETC, oxidative phosphorylation, mitochondrial biogenesis are increased, PPARα and/or ACOX1 expression or activity is increased, GSS and/or GPX4 is insufficiently upregulated, and/or BRAF gene amplification is detected.

The methods may further comprise administration of an additional therapy. The additional therapy may comprise an immunotherapy. In some aspects, the immunotherapy comprises adoptive T cell transfer. In some aspects, the additional therapy comprises an immune checkpoint inhibitor. The immune checkpoint inhibitor may comprise one or both of an anti-PD-1 antibody and an anti-CTLA4 antibody. The additional therapy may comprise a MAPK inhibitor. In some aspects, the MAPK inhibitor comprises a B-Raf inhibitor. In some aspects, the MAPK inhibitor comprises a dual combination of a B-Raf inhibitor and a MEK inhibitor. The additional therapy may also comprise one or more therapies described herein.

The ferroptosis-inducing agent may comprise a GPX4 inhibitor. In some aspects, the ferroptosis-inducing agent comprises one or more of erastin, sulfazine, and RSL3. In some aspects, one or more of these ferroptosis-inducing agents are excluded.

The melanoma cells may be further defined as dedifferentiated or as having a neural crest phenotype. The melanoma cells may have an undifferentiated phenotype. In some aspects, the subject has been previously treated for melanoma with a prior treatment. In some aspects, the prior treatment comprises a MAPK inhibitor. The MAPK inhibitor may comprise a B-Raf inhibitor. The MAPK inhibitor may comprise a dual combination of a B-Raf inhibitor and a MEK inhibitor. The B-Raf inhibitor may comprise vemurafenib or dabrafenib. The MEK inhibitor may comprise selumetinib or trametinib. The MAPK inhibitor may also be one described herein. In some aspects, the prior treatment comprises an immunotherapy. In some aspects, the immunotherapy is one described herein. In some aspects, the prior treatment comprises an additional agent described herein. In some aspects, the subject has been determined to be resistant to the prior treatment.

In some aspects, the melanoma comprises dedifferentiated melanoma or amelanotic melanoma. The subject may be one that has been diagnosed with melanoma. In some aspects, the patient has been diagnosed with dedifferentiated melanoma or amelanotic melanoma. The subject may be one that has been determined to have or has been evaluated as having amplified BRAF gene; increased MITF expression; and/or increased PGC-1α activity or expression, increased TCA cycle, ETC, oxidative phosphorylation, and/or mitochondrial biogenesis; increased PPARα and/or ACOX1 expression or activity; and/or insufficient upregulation of GSS and/or GPX4. The methods may further comprise determining one or more of BRAF gene amplification, MITF expression or activity, PGC-1α activity or expression, and combinations thereof.

In some aspects, the biological sample comprises cancerous cells. In some aspects, the biological sample comprises cancerous skin cells. In some aspects, the level of a biomarker is differentially expressed compared to a control. The control may comprise a non-cancerous sample, a MAPK inhibitor-sensitive cancerous sample, or an immunotherapy-resistant sample. In some aspects, the compositions of the disclosure excludes iron chelators and/or antioxidants.

The methods may comprise or further comprise comparing the expression level of the biomarker to a control. The methods may comprise or further comprise classifying the subject as having ferroptosis-inducer sensitive melanoma when BRAF gene is amplified; MITF expression or activity is increased, and/or PGC-1α expression or activity is increased, one or more of TCA cycle, ETC, oxidative phosphorylation, mitochondrial biogenesis are increased; PPARα and/or ACOX1 expression or activity is increased; and/or GSS and/or GPX4 is insufficiently upregulated. The methods may comprise or further comprise treating the subject classified as ferroptosis-inducer sensitive with a composition comprising a ferroptosis-inducing agent

Detecting the expression level in the biological sample from the subject may comprise determining the mRNA or protein expression of the one or more biomarkers. Determining the level of expression or determining gene amplification may comprise performing fluorescence in situ hybridization (FISH), enzyme-linked immunosorbent assay (ELISA), comparative genomic hybridization (CGH), real time PCR, southern blot, western blot analysis, microarray analysis, or immunohistochemistry. In some aspects, the method further comprises treating the subject diagnosed with melanoma with a composition comprising a ferroptosis-inducing agent.

The administration may be intra-tumoral, intravenous, peri-tumoral, oral, intra-lesional, or sub-cutaneous. The mode of administration may also be a mode described herein.

Methods for determining expression levels, parsing patient populations, and determining cut-off values are known in the art and may include, for example, a Receiver Operating Characteristic (ROC) curve analysis.

The methods may comprise or further comprise recording the expression level or the prognosis score in a tangible medium. The methods may comprise or further comprise reporting the expression level or the prognosis score to the patient, a health care payer, a physician, an insurance agent, or an electronic system. The methods may comprise or further comprise monitoring the patient for cancer recurrence or metastasis or prescribing a treatment that excludes the previously prescribed treatment. The treatment may be any treatment described herein.

Certain methods may involve the use of a normalized sample or control that is based on one or more cancer samples that are not from the patient being tested. Methods may also involve obtaining a biological sample comprising cancer cells from the patient or obtaining a cancer sample.

In some aspects, the expression level is elevated or reduced relative to a control level of expression. In some aspects, the control level is a mean, an average, a normalized value, or a cut-off value. One skilled in the art would understand that a patient would be predicted to respond to a ferroptosis-inducing agent when the expression level of the measured biomarker(s) in the patient sample is the same, or not significantly different, or within 1 or 2 standard deviations from a control that represents a level in a sample that represents ferroptosis-sensitive cells.

In some aspects, the expression or activity level of a protein is determined or has been determined from a biological sample from a patient or a control. The sample may be obtained from a biopsy from the tissue by any of the biopsy methods described herein or known in the art. The sample may be obtained from any of the tissues provided herein that include but are not limited to gall bladder, skin, heart, lung, pancreas, liver, muscle, kidney, smooth muscle, bladder, intestine, brain, prostate, esophagus, or thyroid tissue. In some aspects, the sample may include but not be limited to blood, serum, sweat, hair follicle, buccal tissue, tears, menses, urine, feces, or saliva. In some aspects, the sample may be a tissue sample, a cell free DNA sample, a whole blood sample, a urine sample, a saliva sample, a serum sample, a plasma sample, a skin sample or a fecal sample. In some aspects, the sample comprises cell free DNA.

The methods may further involve isolating nucleic acids such as ribonucleic or RNA or DNA from a biological sample or in a sample of the patient. Other steps may or may not include amplifying a nucleic acid in a sample and/or hybridizing one or more probes to an amplified or non-amplified nucleic acid. The methods may further comprise assaying nucleic acids in a sample. Further aspects include isolating or analyzing protein expression in a biological sample for the expression of polypeptides and biomarkers described herein.

In certain aspects, a microarray may be used to measure or assay the level of protein expression in a sample. The methods may further comprise recording the expression or activity level in a tangible medium or reporting the expression or activity level to the patient, a health care payer, a physician, an insurance agent, or an electronic system.

In some aspects, methods will involve determining or calculating a prognosis score based on data concerning the expression or activity level of one or more genes, meaning that the expression or activity level of a gene is at least one of the factors on which the score is based. A prognosis score will provide information about the patient, such as the general probability whether the patient is sensitive to a particular therapy or has poor survival or high chances of recurrence. In certain aspects, a prognosis value is expressed as a numerical integer or number that represents a probability of 0% likelihood to 100% likelihood that a patient has a chance of poor survival or cancer recurrence or poor response to a particular treatment.

In some aspects, the prognosis score is expressed as a number that represents a probability of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% likelihood (or any range derivable therein) that a patient has a chance of poor survival or cancer recurrence or poor or favorable response to a particular treatment. Alternatively, the probability may be expressed generally in percentiles, quartiles, or deciles.

A difference between or among weighted coefficients or expression or activity levels or between or among the weighted comparisons may be, be at least or be at most about 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000 times or -fold (or any range derivable therein).

In some aspects, the GSH/GSSG ratio is determined to be, is evaluated as, or is measured as or as less than 20, 15, 10, 9, 8, 7, 6, 5, 4, 3, 2, or 1, or any derivable range therein.

In some aspects, determination of calculation of a diagnostic, prognostic, or risk score is performed by applying classification algorithms based on the expression values of biomarkers with differential expression p values of about, between about, or at most about 0.005, 0.006, 0.007, 0.008, 0.009, 0.01, 0.011, 0.012, 0.013, 0.014, 0.015, 0.016, 0.017, 0.018, 0.019, 0.020, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.040, 0.041, 0.042, 0.043, 0.044, 0.045, 0.046, 0.047, 0.048, 0.049, 0.050, 0.051, 0.052, 0.053, 0.054, 0.055, 0.056, 0.057, 0.058, 0.059, 0.060, 0.061, 0.062, 0.063, 0.064, 0.065, 0.066, 0.067, 0.068, 0.069, 0.070, 0.071, 0.072, 0.073, 0.074, 0.075, 0.076, 0.077, 0.078, 0.079, 0.080, 0.081, 0.082, 0.083, 0.084, 0.085, 0.086, 0.087, 0.088, 0.089, 0.090, 0.091, 0.092, 0.093, 0.094, 0.095, 0.096, 0.097, 0.098, 0.099, 0.1, 0.2, 0.3, 0.4, 0.5, 0.6, 0.7, 0.8, 0.9 or higher (or any range derivable therein). In certain aspects, the prognosis score is calculated using one or more statistically significantly differentially expressed (increased or decrease) biomarkers (either individually or as difference pairs), including expression or activity levels in a gene or protein.

Any of the methods described herein may be implemented on tangible computer-readable medium comprising computer-readable code that, when executed by a computer, causes the computer to perform one or more operations. In some aspects, there is a tangible computer-readable medium comprising computer-readable code that, when executed by a computer, causes the computer to perform operations comprising: a) receiving information corresponding to an expression or activity level of a gene or protein in a sample from a patient; and b) determining a difference value in the expression or activity levels using the information corresponding to the expression or activity levels in the sample compared to a control or reference expression or activity level for the gene.

In other aspects, tangible computer-readable medium further comprise computer-readable code that, when executed by a computer, causes the computer to perform one or more additional operations comprising making recommendations comprising: wherein the patient in the step a) is under or after a first treatment for cancer, administering the same treatment as the first treatment to the patient if the patient does not have increased expression or activity level; administering a different treatment from the first treatment to the patient if the patient has increased expression or activity level.

In some aspects, receiving information comprises receiving from a tangible data storage device information corresponding to the expression or activity levels from a tangible storage device. In additional aspects the medium further comprises computer-readable code that, when executed by a computer, causes the computer to perform one or more additional operations comprising: sending information corresponding to the difference value to a tangible data storage device, calculating a prognosis score for the patient, treating the patient with a traditional therapy if the patient does not have expression or activity levels, and/or or treating the patient with an alternative therapy if the patient has increased expression or activity levels.

The tangible, computer-readable medium further comprise computer-readable code that, when executed by a computer, causes the computer to perform one or more additional operations comprising calculating a prognosis score for the patient. The operations may further comprise making recommendations comprising: administering a treatment comprising a ferroptosis or other cell death-inducing agent to a patient that is determined to have a particular phenotype or biomarkers expression level.

The subject may be a human, mouse, pig, cow, sheep, rabbit, or rat. In some aspects, the subject is a non-human primate. In some aspects, the subject is a human or a mammal.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments are discussed throughout this application. Any embodiment discussed with respect to one aspect of the disclosure applies to other aspects of the disclosure as well and vice versa.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-J. Focal amplifications in the form of DMs and HSRs mediate resistance to BRAF+MEK inhibition. A, BRAFi+MEKi treatment history for M249 cells. Dots on the line represents rough sample collection points at three stages. B, FISH images show three different karyotypes coming from corresponding time points in (A) with number of observations labeled below. Red: BRAF. Green: centromere 7. Blue: DAPI. C, qPCR results of relative BRAF copy number in the samples from three time points in (A). Error bars represent t-distribution based 95% confidence intervals (see Method). CN: copy number. RQ: relative quantity. n=3. D, Immunoblot of BRAF for all three corresponding samples in (A). E-F, Whole genome sequencing results show that the most significant copy number increase in M249-VSR-DM and -HSR takes place at 7q34. Gene annotations within the amplicon were obtained from UCSC genome browser. G, mRNA level of genes that are on the amplicon of M249-VSR, measured by RNAseq. TPM: transcript per million. H, Frequencies of c.1799T>A (V600E) in M249-P, -VSR-DM and -VSR-HSR cells, inferred by aligning RNA-seq reads to the genome. MAF: major allele frequency. Green: thymine. Red: adenine. I, Bionano optical mapping results of BRAF regions in M249-P and M249-VSR-DM show the latter sample has closed circular structure for BRAF amplicon. J, G-banding for M249-VSR-HSR bulk cells shows HSRs are located on three different chromosomes. The frequency of each category is in parenthesis. M: acrocentric marker chromosome.

FIG. 2A-G. Single-cell-derived clones reveal de novo integrations of DMs into chromosomes as HSRs. A, The timeline of deriving M249-VSR SCs with sampling points for BRAF FISH assays indicated. B-x: M249 bulk cells at different time points. SCx-B: freshly derived SCs before three-month culture. SCx-A: derived SCs after three-month culture. B, FISH images of sampling points for both bulk and SC samples in (A). C-D, Karyotype percentages for sampling points in (A). E, SC4 DM+ cells were maintained in either DMSO (90 days) or DNA-PK inhibitor 5 μM NU7026 (107 days). FISH was then performed to assess presence of HSRs in each cell. 100 cells were examined in each replicate. P-value is based on one-tailed Welch's t-test (n=3). F, G-banding of subclone SC2 shows HSR located on Chr3. Ratio represents the number of metaphases of such HSR chromosomal location divided by the number of all metaphases examined. G. SC2 cells were maintained in either DMSO or 5 μM NU7026 for three months. Then FISH was carried out to detect number of HSRs in each cell. Values on the top of bars are numbers of cells examined. P-value is calculated by one-tailed Welch's t-test (n=3).

FIG. 3A-L. A variety of focal amplifications modes and secondary resistance mechanisms mediate dynamic plasticity to BRAF and MEK inhibition. A, The treatment history of various experiments on bulk M249 VSR cells with labels of time points for when cells were fixed (FIX) for FISH and their genomic DNA (gDNA) were extracted. Top bar shows the estimated duration of each stage, inferred from FIG. 2C. P-DEV: resistance developmental stage from M249 parental cells. DM: the stage when the karyotype is predominantly DM+ & HSR−. HSR: the stage when the karyotype is predominantly DM− & HSR+. Grey dots represent common time points between different experiments. B, Representative FISH images of fixation points in (A). Images are only shown if the corresponding karyotypes occurred at high frequencies. C, Full karyotype percentages of samples in (A). DM− & HSR+(M): multiple HSRs; DM− & HSR+(S): short HSRs. D, Representative qPCR results of BRAF copy number for some gDNA extraction points in (A). n=3. E, FISH images of M249-VSR-DM bulk cells cultured for one month with single or both drugs withdrawn. F, Per cell DM counts of samples in (E). P-values were calculated by two-tailed t test, G, FISH images of parental stage, resistance stage and the stage after a long-term culture of resistance cells at reduced dose for A375 and Me1888 cell lines. P: parental. DTR: dabrafenib (DAB)+trametinib (TRA) resistance. LC: long-term culture. H, The ratios of BRAF and DAPI stain areas of samples in (G) were measured as a semi-quantification method for BRAF HSR sizes. P-values are based on one-tailed Wilcox tests. I-L, FFPE FISH and statistics of PDX models PDX1 (NRAS^{Q61R}) and PDX13 (BRAF^{S365L}) as well as FISH and statistics of their derived cell lines for the stages after acquiring resistance to Trametinib or after drug withdrawal. PDX samples were fixed when tumor relapsed from perturbations. Number of metaphases analyzed are labeled on the right side of the bars in (J). P values in (L) were calculated using two-tailed t test. TRA: trametinib. V: vehicle. TR: trametinib resistance. DW: drug withdrawal. Amp: amplification. CL: cell line. FFPE: formalin-fixed paraffin-embedded.

FIG. 4A-K: The plasticity of BRAF amplification is reproducible at single cell level, supporting de novo genomic changes in addition to selection. A, Representative FISH images of three SCs that were treated either with 2 μM (original dose) or 0.1 μM VEM+SEL for roughly three months. LS: long and short HSR in one cell. L: long HSR. S: short HSR. B, qPCR of samples in (A). n=3. B-: before three-month culture. A-: after three-month culture. C, The full percentage of each karyotype for samples in (A). D, Cell number measurements after VEM+SEL was withdrawn from M249-VSR bulk cells and SCs. Error bars are standard deviations from three technical replicates. Predominant BRAF FA modes are denoted in parenthesis. E, FISH images of M249 SC2 before and after VEM+SEL dose reduced from 2 μM to 0.1 μM or kept at 2 μM using BRAF and chromosome 3 centromere probes. F, A summary of what size of HSR is on chromosome 3 in each cell before and after VEM+SEL dose reduction. Number of metaphases analyzed are on top of the bars. G, A summary of whether long and short HSRs are on chromosome 3 or other chromosomes before and after VEM+SEL dose reduction. H, Model of BRAF amplicon HSR integration structure inferred by optical mapping data before and after VEM+SEL dose reduction. Letters represent distinct junctions, as summarized in Supplementary Table S1 and FIG. 17. Purple boxes represent probable repeat units. Note that variations of both the before and after models are possible, such as an additional S junction plus the 139,518K-141,069K amplicon segment being inserted anywhere that an S junction is located. C1-I and C2-C3-I repeats are interchangeable. The C2 junction need not be present in some of the C2-C3-I repeats. I, FISH images with BRAF and PAK2 probes supporting the structure in (H). J, RAF1 or NRAS FISH images of two M245 (NRAS^{Q61K}) SCs upon becoming resistant to trametinib, and upon recovery from trametinib withdrawal. P: parental. TRA: trametinib. TR: Trametinib resistance. K, Frequencies of karyotypes for samples in (J). Number of metaphases analyzed are on the top of the bars.

FIG. 5A-L. HSR to DM karyotypic switching and BRAF kinase domain duplications mediate resistance to MAPK inhibitor dose increase. A, The relationship between samples examined during the processes of M249 VSR development and VEM+SEL 2 μM to 5 μM dose increases. B, Representative FISH images of all samples in (A). C, The frequencies of karyotypes for samples in (A). D, Immunoblot of samples in (A), using an antibody that targets the N-terminus of BRAF (12-156aa). The 140 kD band is the KDD form, and the 62 kD band is the alternatively spliced form of BRAF. E-F, qPCR and RT-PCR for samples in (A) with primer sets that target BRAF exon 18-10 and exon 9-10 junctions. For RT-qPCR, all values of exon junction 18-10 were normalized to that of exon junction 9-10 of corresponding samples. Error bars represent SEMs around ΔCt values derived by Satterthwaite approximation. G, M249-VSR-HSR bulk cells were sorted into single cells on day 322 of the timeline in panel A and seeded in 96-well plates. Cells were next treated with either original 2 μM (n=3) or 5 μM VEM+SEL (n=10) for 12 days. The sizes of the resulting colonies were classified into three categories (Small, Medium and Large) by eye. H, second replica screen for M249-VSR-HSR single-cell-derived clones that tolerate VEM+SEL 2 to 5 μM dose increase. Rows of the heatmap represent different clones ranked by relative growth rate (RGR), calculated by dividing the mean viability at 5 μM by that at 2 μM after a six-day culture. Boxplot shows mean and standard deviations of CellTiter-Glo viability (×1000) for each clone on the sixth day (see method). I, representative FISH images of selected clones in (H) with frequency of each FA mode. J, Immunoblot of BRAF in bulk cells and single-cell-derived clones treated with the indicated dose regiments. K, Design of the barcode-based clone tracing experiments. Cells were transduced with the lentivirus ClonTracer library on day 318 based on the timeline in (A). L, Comparison between barcode fractions on Day 14 and Day 35 as depicted in (K). Top 10 barcodes by fraction from each sampling time point are highlighted.

FIG. 6A-C. BRAF amplicon boundaries are mostly preserved among switching DM, HSR, short HSR and KDD-DM. A, Treatment history of M249 samples that have been profiled by WGS. B, Amplicon Architect results of BRAF amplicon for M249 samples in (A). C, A summary of amplification frequencies of regions around BRAF in MAPKi-treated post-progression melanoma samples from previous reports. Solid line represents percentage of samples that pass a BRAF CN log 2(post/normal) threshold. Dashed line represents expected frequencies for a single locus of selection (see Methods). Heatmap shows CNA data of all samples analyzed at the same Chr 7 region.

FIG. 7A-C. Melanoma cell lines with acquired dual BRAFi+MEKi resistance through BRAF amplification mechanism show sensitivity to ferroptosis inducing agent. A, Dose-response curves showing increased sensitivity to RSL3 in 3 cases of dual BRAFi+MEKi resistance mediated by BRAF amplification (M249-VSR (both DM and HSR modes of amplification), 888mel-DTR, and A375-DTR) compared to parental sublines. Upon drug withdrawal (DW), the sensitivity of M249-VSR revert to be closer to the original parental case. Three or six replicates. 72 hr treatment. B-C, Measurements of percent viable cells with DMSO, RSL3 alone or in combination with the antioxidant reduced glutathione (GSH) (n=6), the lipophilic antioxidant Trolox (n=3) and the iron chelator DFO (n=3). Two-tailed t-test: ns: p>0.05 *: p≤0.05, **: p≤0.01, ***: p≤0.001, ****: p≤0.0001.

FIG. 8A-C. BRAF FA karyotype categories and subcategories. We divided karyotypes into four primary categories: DM− & HSR−, DM+ & HSR−, DM− & HSR+, and DM+ & HSR+. Some categories have distinguishable sub-categories. A-C, Shown are representative FISH images of each BRAF FA category and sub-category. Some less-frequent sub-categories are not shown here. Red: BRAF. Green: centromere 7. Blue: DAPI.

FIG. 9A-C. BRAF DNA copy number amplification results confirmed by additional methods. Related to FIG. 1. A, Low-pass whole genome sequencing (WGS)-based BRAF and genome-wide copy number results of M249-P and M249-VSR-DM cells. Plotted is the whole genome CNA overview generated by the Ginkgo software. Below are the zoomed-in plots at the BRAF locus. Copy number values at the positions indicated by the green dots are shown in the inset boxes. B, Comparative genome hybridization (CGH) results of M249-P and M249-VSR-DM cells. The circled region highlights the BRAF focal amplicon on chromosome 7q in M249-VSR cells. C, CNA of chr7 in M249-P and M249-VSR-DM cells inferred by Bionano optical mapping (OM). X axis: genomic coordinates. Y axis: absolute copy number.

FIG. 10A-D. Single-cell-derived clone SC401 displays DM amplicon with circular structure, with subsequent chromosomal integration as an HSR. Related to FIG. 2. The SC401 clone derived from M249-VSR bulk cells was contaminated by mycoplasma during long term culture. However, it was mycoplasma negative at the time it was freshly derived. Due to this contamination, we only display data from this clone in a minimal number of supplemental items (this figure, FIG. 11B, and Supplementary Table S1). All conclusions made in the manuscript stand independent of this clone. Nevertheless, this clone remains highly consistent with the other findings, including amplicon structure and integration properties, and thus we present its data. A-B, Example images and karyotype frequencies of SC401 before and after 3-month culture at the constant VEM+SEL dose 2 μM. -B: before. -A: after. C-D, BRAF circular amplicon structure in SC401 inferred by optical mapping data, with a similar inferred ecDNA structure to that in bulk M249-VSR-DM cells. S is the circle closing junction.

FIG. 11A-B. BRAF amplification in DM mode decreased its copy number in single-cell-derived clones (SCs) before and after three-month culture. Related to FIG. 2. A-B, Relative quantity (RQ) of BRAF copy number (CN) before and after long term culture at constant dose, calculated by averaging multiple independent qPCR runs (n represents number of replicates). Error bars were calculated using propagation of errors. See notes on SC401 in FIG. 10 legend.

FIG. 12A-D. Bulk MAPK inhibitor resistant melanoma cells displayed an increase in growth rate over time, while SCs showed varying degrees of change in growth rate. Related to FIG. 2. The M249 VSR bulk population increased their proliferation rate over the three-month culture. Two DM only (SC3, SC4) and one DM plus HSR (SC5) clones also displayed continuously increased proliferation rates (decreased doubling times whereas the HSR only clone (SC2) did not increase its proliferation rate. 0.05 million cells were plated in each well of 12-well plates, and cell numbers were monitored for a maximum of 12 days. Data points were fitted to the exponential growth curve y=y0·ekx, where y0 is the initial cell number, i.e. 0.05 million, y is the cell number at time x, and k is the rate constant. Three technical replicates for each time point. A, Bulk M249-VSR cells. The days since establishment as a resistant subculture is indicated in the legend as −xxxD. B, SCs shortly after single cell clone establishment. C, SCs after 3 months of culture. D, The summary of changes on doubling times for M249-VSR bulk and SC cells over time. Error bars represents standard error of means (SEMs) of doubling times, n=3 (see Method).

FIG. 13A-C. Treating DM+ cells with oscillating doses of BRAF and MEK inhibitors conferred a selection advantage for the DM+ & HSR− subpopulation. Related to FIG. 3. A, Oscillating (OSCI) and steady dose (CTRL) treatment schemes of M249-VSR-DM cells using VEM+SEL. CTRL is a DM to HSR transition control similar to FIG. 3A EXP1 FIX3. B, Representative FISH images for the sampling points indicated in (A). In the steady dose case, most observed cells were HSR positive on day 246, but in the oscillating dose case there were no detected HSR positive cells even approximately two months later on day 308. C, Western blot results for M249 Parental sample and M249-VSR with oscillating dose (labeled in A).

FIG. 14A-C. Double drug withdrawal eliminated BRAF-carrying DMs in about 15 days. Related to FIG. 3. A, Treatment scheme of M249 cells with VEM+SEL. Points shown represent when cells were fixed (FIX) and collected for genomic DNA (gDNA). B, qPCR results of relative BRAF copy number for the time points in (A). CN: copy number. RQ: relative quantity. C, Representative metaphase spread images and FISH images for the time points in (A).

FIG. 15A-B. M249-VSR DM+ cells tolerate single-drug withdrawal better than HSR+ cells, but there is no difference on recovery rate between DM and HSR cells for double-drug withdrawal. Related to FIG. 3. A, Short term viability and growth rates for M249-VSR-DM and HSR bulk cells upon acute withdraw of one of or both MAPK inhibitors. Viability was measured by the CellTiter-Glo (CTG) Luminescent assay. B, Long term growth rate measurement for the same treatments in (A). Expected cells counts were calculated by multiplying together all cell number fold changes (measured upon each passage).

FIG. 16A-C. VEM+SEL dose reduction caused BRAF HSR length to shrink in SCs. Related to FIG. 4. A, Normalized BRAF probe area in FISH images before and after dose reduction for quantifying HSR lengths. P-values are based on one-tailed Wilcox test. B, Representative FISH images of the DM− & HSR+ clone SC302 before and after dose reduction. C, Karyotype frequencies of clone SC302 before and after dose reduction. S: short HSR. L: long HSR. -B-: Before, -A-: After.

FIG. 17A-D. DM− & HSR+ subclone SC2 show alternative BRAF amplicon structure, and its integration on chr3 is supported by PAK2 amplifications. The integration junctions stayed unchanged upon the VEM+SEL dose reduction. Related to FIG. 4. A, Optical mapping-inferred junctions used to build the model of the SC2 HSR genomic structure in FIG. 4, as well as the S junction shown in FIG. 10D. The number of observed optical mapping supports for these junctions are summarized in Supplementary Table S1. B, CNA callings by WGS for multiple M249-VSR variants in this article show DM− & HSR+ subclone SC2 has PAK2 amplification. Its dose reduced version (SC2-2-0.1) and bulk DM− & HSR+ population (M249-VSR-HSR) have weaker and heterogenous PAK2 amplifications. C, CNA calling by optical mapping for SC2 before and after dose reduction show decrease of BRAF copy number and PAK2 amplification around chr3 telomere prior to the dose reduction. D, BRAF HSR integration junction between chr7 and chr3 before and after dose reduction, revealed by SVABA analysis using WGS data. FIG. 17 shows SEQ ID NOS:5, 5, 6, 7, 8, 9, 10, 11, 12, 12, 13, 14, 15, 16, 17, 18, 5, 5, and 5, respectively (left) and SEQ ID NOS:5, 5, 5, 19, 20, 21, 22, 23, 24, 18, 5, and 19, respectively (right)

FIG. 18A-C. Treatment of M395 melanoma cells with MAPK inhibitors led to BRAF amplification on HSRs co-occurring with BRAF kinase domain duplication. HSR length did not decrease upon drug withdrawal in this case. Related to FIG. 4. A, VEM+SEL treatment scheme starting from 0.05 μM on M395-P (parental) cells. The points when cells were collected for genomic DNA (gDNA), fixation (FIX) and protein lysates (LYSATE) are labeled. B, Representative FISH images for fixation time points in (A). C, qPCR results of relative BRAF copy number for gDNA collection points in (A). CN: copy number. RQ: relative quantity. D, western blot for lysate collection time points in (A).

FIG. 19A-C. Drug dose challenge characterization of single-cell-derived clones. Related to FIG. 5. A, Experimental design to generate single-cell-derived clones (SC1XXs) by sorting M249-VSR-HSR bulk cells on day 322, followed by two rounds of replica screens. B, As depicted in (A), acute 2 to 5 μM VEM+SEL treatment on 41 SC1XXs was used to screen for clones that adapt to 5 μM rapidly. The rows of the heatmap represent different SC1XXs ordered by relative growth rate (RGR), calculated by dividing the mean at 5 μM by that at 2 μM, in descending order. Viability was measured by CellTiter-Glo, and the readings were divided by 1000 followed by capping at 50. C, Representative FISH images of two SC1XXs at the lower tail of the heatmap in (B).

FIG. 20. The DM+ and KDD+ single-cell-derived clones SC101 and SC137 demonstrate the best ability to tolerate MAPK inhibitor dose increases, compared to other SC1XXs. Related to FIG. 5. The indicated M249-VSR subclones and M249-VSR-HSR bulk cells initially cultured at 2 μM of VEM+SEL were treated with various subsequent inhibitor doses for 4 days, and then their viabilities were measured. All numbers are normalized to the corresponding viabilities at 2 μM. p-values are based on one-tailed t test (n=6).

FIG. 21A-B. MAPK inhibitor dose escalation applied to HSR-positive SCs did not result in the DM+ & KDD+ genomic configuration. Related to FIG. 5. A, Representative FISH pictures of the DM− & HSR+M249-VSR SCs, SC2 and SC208, with VEM+SEL dose escalated from 2 μM to 5 μM until they became resistant. B, Immunoblot of BRAF samples in (A) showing no 140 kDa KDD band after the VEM+SEL dose increase.

FIG. 22. The pre-treatment BRAF copy number does not predict the increase of BRAF copy number upon resistance to MAPKi. Correlation between BRAF copy number before MAPKi treatment and the BRAF copy number increase after relapsing from the treatment in melanoma. R2 is the squared sample Pearson correlation coefficient, and the correlation P-value is based on the t-distribution.

FIG. 23A-I. The ferroptosis sensitivity of melanoma cells with BRAF amplification as dual MAPKi resistance mechanism is not due to dedifferentiation. Related to FIG. 7. A, Lipid ROS in M249-P and M249-VSR-DM measured by flow cytometry using lipophilic ROS-sensitive BODIPY™ 581/591 C11 dye upon treatment with or without 1 μM RSL3 and 150 μM Trolox for 24 hr, demonstrating that the lipophilic antioxidant Trolox protects against RSL3-induced lipid ROS. B, Dose-response curve showing increased sensitivity to ferroptocide in in BRAFi+MEKi resistance mediated by BRAF amplification (M249-VSR-DM and -HSR) but no differential sensitivity to Erastin compared to parental cells. Cell viabilities were measured by CellTiter-Glo. Three or six replicates. 72 hr treatment. Each experiment was repeated twice. C, Projections of the M249-P and M249-VSR variant samples from the current manuscript onto the differentiation trajectory (transcriptomic principal component analysis (PCA)) of the M series of melanoma cell lines from Tsoi et al(1). The four melanoma differentiation stages are indicated. All M249-P and M249-VSR variants start and remain in the differentiated (melanocytic) cluster upon acquisition of MAPKi resistance. In our past studies, melanoma cells that develop MAPKi resistance through genomic changes that reactivate the MAPK signaling pathway do not dedifferentiate, e.g. M249P/R (NRAS mutation-mediated resistance in this version of single agent BRAFi resistance), do not show different sensitivity to ferroptosis inducing agents, while other cases of resistance due to dedifferentiation (also featured by receptor tyrosine kinase upregulation) are observed, e.g. M229P/R and M238P/R(1,2). Note that our M249-P and M249-VSR BRAF amplification lines are projected at the same location as the independently derived case of resistance (M249R) and its parental (M249P) pair. In this case resistance is to single agent BRAFi (vemurafenib), with resistance mediated by NRAS mutation(1,2). Notably, the BRAF-amplified M249-VSR cells are sensitive to RSL3 (FIG. 7A), unlike the NRAS-mutant M249R case(1). D, The same reference PCA-based differentiation state spectrum as in (A), with projections of Mel888-P/-DTR (BRAF amplification), A375-P/-DTR (BRAF amplification) and SKMEL28P/R (dedifferentiation) cell lines. BRAF amplification mediated resistant sublines do not demonstrate gene expression-based signatures of dedifferentiation as compared to their parental pairs. Data was downloaded from the corresponding papers(3-8). E-F, mRNA expression and single sample GSEA (ssGSEA)(9) of selected genes and gene sets in the melanoma cell lines before and after establishment of resistance to MAPK inhibitors. Y: YES. N: NO. Amp: amplification. Mut: mutation: RTK Up: receptor tyrosine kinase upregulation. HSRR: higher sensitivity to RSL3 in resistance line. Dediff: dedifferentiation upon resistance. Log 10 counts per million (CPM) and ssGSEA z scores were calculated by standardizing within each gene, and for the visualization the values were capped from −2 to +2. G-H, selected gene mRNA levels and mRNA-based ssGSEA scores for cell lines in the M series. I, Glutathione levels, reduced (GSH), oxidized (GSSG), and ratio (GSH/GSSG), in M249 sublines measured by mass spectrometry. p-values were calculated using one-tailed t test.

FIG. 24A-D. Melanoma cell lines with acquired dual BRAFi+MEKi resistance through BRAF amplification mechanism show sensitivity to ferroptosis inducing agent. A, Dose-response curves showing increased sensitivity to RSL3 in 3 cases of dual BRAFi+MEKi resistance mediated by BRAF amplification (M249-VSR (both DM and HSR modes of amplification), 888mel-DTR, and A375-DTR) compared to parental sublines. Upon drug withdrawal (DW), the sensitivity of M249-VSR revert to be closer to the original parental case. Three or six replicates. 24 hr treatment. Cell viabilities were measured by CellTiter-Glo. B-C, Measurements of percent viable cells with DMSO, RSL3 alone or in combination with the antioxidant reduced glutathione (GSH) (n=6), the lipophilic antioxidant Trolox (n=3) and the iron chelator DFO (n=3). T-test: ns: p>0.05 *: p≤0.05, **: p≤0.01, ***: p≤0.001, ****: p≤0.0001. D, Dose-response curves showing no substantial change in sensitivity to Erastin in BRAFi+MEKi resistance mediated by BRAF amplification (M249-VSR (both DM and HSR modes of amplification)) compared to parental sublines. Six replicates.

FIG. 25A-H. The ferroptosis sensitivity of melanoma cells with BRAF amplification as dual MAPKi resistance mechanism is not due to dedifferentiation. A, Lipid ROS in M249-P and M249-VSR-DM measured by flow cytometry using lipophilic ROS-sensitive BODIPY™ 581/591 C11 dye upon treatment with or without 1 μM RSL3 and 150 μM Trolox for 24 hr, demonstrating that the lipophilic antioxidant Trolox protects against RSL3-induced lipid ROS. B, Projections of the M249-P and M249-VSR variant samples from the current manuscript onto the differentiation trajectory (transcriptomic principal component analysis (PCA)) of the M series of melanoma cell lines from Tsoi et al (1). The four melanoma differentiation stages are indicated. All M249-P and M249-VSR variants start and remain in the differentiated (melanocytic) cluster upon acquisition of MAPKi resistance. In past studies, melanoma cells that develop MAPKi resistance through genomic changes that reactivate the MAPK signaling pathway do not dedifferentiate, e.g. M249P/R (NRAS mutation-mediated resistance in this version of single agent BRAFi resistance), do not show different sensitivity to ferroptosis inducing agents, while other cases of resistance due to dedifferentiation (also featured by receptor tyrosine kinase upregulation) are observed, e.g. M229P/R and M238P/R (31). Note that the M249-P and M249-VSR BRAF amplification lines are projected at the same location as the independently derived case of resistance (M249R) and its parental (M249P) pair. In this case resistance is to single agent BRAFi (vemurafenib), with resistance mediated by NRAS mutation (31). Notably, the BRAF-amplified M249-VSR cells are sensitive to RSL3 (FIG. 24A), unlike the NRAS-mutant M249R case (1). C, The same reference PCA-based differentiation state spectrum as in (A), with projections of Mel888-P/-DTR (BRAF amplification), A375-P/-DTR (BRAF amplification) and SKMEL28P/R (dedifferentiation) cell lines. BRAF amplification mediated resistant sublines do not demonstrate gene expression-based signatures of dedifferentiation as compared to their parental pairs. Data was downloaded from the corresponding papers (2, 22-26). D-E, mRNA expression and single sample GSEA (ssGSEA) (27) of selected genes and gene sets in the melanoma cell lines before and after establishment of resistance to MAPK inhibitors. Y: YES. N: NO. OE: overexpression. Amp: amplification. Mut: mutation: RTK Up: receptor tyrosine kinase upregulation. HSRR: Higher sensitivity to RSL3 in resistance line. Dediff: dedifferentiation upon resistance. Log 10 counts per million (CPM) and ssGSEA z scores were calculated by standardizing within each gene, and for the visualization the values were capped from −2 to +2. F-G, selected gene levels and gene set ssGSEA scores for cell lines in the M series of panel B. H, Glutathione levels, reduced (GSH), oxidized (GSSG), and ratio (GSH/GSSG), in M249 sublines measured by mass spectrometry. P values were calculated using one-tailed t test.

FIG. 26. Evidence for increased ferroptosis sensitivity in BRAF amplified cells form knockout of GPX4. CRISPR knockout of the oxidized lipid repair enzyme glutathione peroxidase 4 (GPX4) results in decreased cell viability in BRAF amplified cells. This effect was more emphasized in the cells with the homogeneously staining region (HSR) mode of BRAF amplification. Results shown in the other figures confirmed that ferroptosis sensitivity is also present in BRAF amplified cells with the extrachromosomal DNA (ecDNA) mode of BRAF amplification. Vulnerability to knockout or knockdown of GPX4 is highly correlated to sensitivity to pro-ferroptotic drugs, including drugs that target GPX4. Gene effect was calculated by summarizing both log fold change of barcode abundance and its p value before and after 14 or 21 days of cell culture post lentivirus infection and selection in a CRISPR screen.

### DESCRIPTION OF ILLUSTRATIVE EMBODIMENTS

Ferroptosis occurs through an iron-dependent accumulation of lethal lipid reactive oxygen species (ROS) and regulated by GPX4, a glutathione-dependent enzyme that catalyzes the reduction of lipid ROS to lipid alcohols (Dixon et al., 2012; Yang et al., 2014). Ferroptosis is a relatively recent discovery of programmed cell death distinct from apoptosis and it was unexpectedly discovered that inducing ferroptosis in patients with certain genetic changes can enhance signaling inhibition and immune therapies by synthetic lethal induction of ferroptosis.

### I. Definitions

As used herein, the term “antibody” encompasses antibodies and antibody fragments thereof, derived from any antibody-producing mammal (e.g., mouse, rat, rabbit, and primate including human), that specifically bind to an antigenic polypeptide. Exemplary antibodies include polyclonal, monoclonal and recombinant antibodies; multispecific antibodies (e.g., bispecific antibodies); humanized antibodies; murine antibodies; chimeric, mouse-human, mouse-primate, primate-human monoclonal antibodies; and anti-idiotype antibodies, and may be any intact molecule or fragment thereof.

The term “sensitivity” or “sensitive” in the context of an agent, such as a ferroptosis inducing agent, acting on a cell, such as a cancer cell, refers to the agent's ability to lyse or kill the cell. For example, cells sensitive to ferroptosis-inducing agents refers to cells that are killed and/or lysed by said ferroptosis-inducing agent.

The term substantially the same or not significantly different refers to a level of expression that is not significantly different than what it is compared to. Alternatively, or in conjunction, the term substantially the same refers to a level of expression that is less than 2, 1.5, or 1.25 fold different than the expression or activity level it is compared to.

A “subject,” “individual” or “patient” is used interchangeably herein and refers to a vertebrate, for example a primate, a mammal or a human. Mammals include, but are not limited to equines, canines, bovines, ovines, murines, rats, simians, humans, farm animals, sport animals and pets. Also intended to be included as a subject are any subjects involved in clinical research trials not showing any clinical sign of disease, or subjects involved in epidemiological studies, or subjects used as controls.

The term “primer” or “probe” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

As used herein, “increased expression,” “increased level of expression,” “elevated expression,” “decreased expression,” or “decreased level of expression” refers to an expression level of a biomarker in the subject's sample as compared to a reference level representing the same biomarker or a different biomarker. In certain aspects, the reference level may be a reference level of expression from a non-cancerous tissue from the same subject. Alternatively, the reference level may be a reference level of expression from a different subject or group of subjects. For example, the reference level of expression may be an expression level obtained from a sample (e.g., a tissue, fluid or cell sample) of a subject or group of subjects without cancer, or an expression level obtained from a non-cancerous tissue of a subject or group of subjects with cancer. The reference level may be a single value or may be a range of values. The reference level of expression can be determined using any method known to those of ordinary skill in the art. In some aspects, the reference level is an average level of expression determined from a cohort of subjects with cancer or without cancer. The reference level may also be depicted graphically as an area on a graph. In certain aspects, a reference level is a normalized level.

“About” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. In some aspects it is contemplated that an numerical value discussed herein may be used with the term “about” or “approximately.”

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. “Consisting essentially of” in the context of pharmaceutical compositions of the disclosure is intended to include all the recited active agents and excludes any additional non-recited active agents, but does not exclude other components of the composition that are not active ingredients. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Aspects defined by each of these transition terms are within the scope of this invention.

The terms “protein”, “polypeptide” and “peptide” are used interchangeably herein when referring to a gene product or functional protein.

The terms “ameliorating,” “inhibiting,” or “reducing,” or any variation of these terms, when used in the claims and/or the specification includes any measurable decrease or complete inhibition to achieve a desired result.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

The term “inhibitor” refers to a therapeutic agent that indirectly or directly inhibits the activity or expression of a protein, process (e.g. metabolic process), or biochemical pathway.

A person of ordinary skill in the art understands that an expression level from a test subject may be determined to have an elevated level of expression, a similar level of expression or a decreased level of expression compared to a reference level.

As used herein, “treating,” “treatment” or “therapy” is an approach for obtaining beneficial or desired clinical results. This includes: reduce the alleviation of symptoms, the reduction of inflammation, the inhibition of cancer cell growth, and/or the reduction of tumor size. In some aspects, the term treatment refers to the inhibition or reduction of cancer cell proliferation in a subject having cancer. Furthermore, these terms are intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs. See Johnson et al. (2003) J. Clin. Oncol. 21(7):1404-1411.

The term “therapeutically effective amount” refers to an amount of the drug that treats or inhibits cancer in the subject. In some aspects, the therapeutically effective amount inhibits at least or at most or exactly 100, 99, 98, 96, 94, 92, 90, 85, 80, 75, 70, 65, 60, 55, 50, 40, 30, 20, or 10%, or any derivable range therein, of a protein's activity or expression.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

### II. Therapeutic Agents

### A. Ferroptosis-Inducing Agents

Ferroptosis occurs through an iron-dependent accumulation of lethal lipid reactive oxygen species (ROS) and regulated by GPX4, a glutathione-dependent enzyme that catalyzes the reduction of lipid ROS to lipid alcohols (Dixon et al., 2012; Yang et al., 2014). Ferroptosis is a relatively recent discovery of programmed cell death distinct from apoptosis and the methods and compositions of the current application provides a differentiated-guided approach that can be harnessed to counter a melanoma therapy escape route.

Exemplary ferroptosis-inducing agents include glutathione synthesis inhibitors such as erastin, sulfalazine, buthioninesulfoximine (BSO), sorafenib, and DPI2; GPX4 inhibitors such as RSL3, RSL5, ML162, ML210, DPI7, DPI10, DPI12, DPI13, DPI17, DPI18, DPI19, CIL56, and FIN56; and other agents such as DPI3, DPI4, DPI6, CIL41, CIL69, CIL70, CIL75, and CIL79. Further examples include analogs of the disclosed ferroptosis-inducing agents such as erastin-A, erastin-B, or desmethyl-erastin, and sorafenib analogs, such as those described in WO 2015051149.

### B. Immunotherapies

In some aspects, the methods include the administration of an immunotherapy. Exemplary immunotherapies are described below.

### 1. Checkpoint Inhibitors

An “immune checkpoint inhibitor” is any molecule that directly or indirectly inhibits, partially or completely, an immune checkpoint pathway. Without wishing to be bound by any particular theory, it is generally thought that immune checkpoint pathways function to turn on or off aspects of the immune system, particularly T cells. Following activation of a T cell, a number of inhibitory receptors can be upregulated and present on the surface of the T cell in order to suppress the immune response at the appropriate time. In the case of persistent immune stimulation, such as with chronic viral infection, for example, immune checkpoint pathways can suppress the immune response and lead to immune exhaustion. Examples of immune checkpoint pathways include, without limitation, PD-1/PD-L1, CTLA4/B7-1, TIM-3, LAG3, By-He, H4, HAVCR2, ID01, CD276 and VTCN1. In the instance of the PD-1/PD-L1 immune checkpoint pathway, an inhibitor may bind to PD-1 or to PD-L1 and prevent interaction between the receptor and ligand. Therefore, the inhibitor may be an anti-PD-1 antibody or anti-PD-L1 antibody. Similarly, in the instance of the CTLA4/B7-1 immune checkpoint pathway, an inhibitor may bind to CTLA4 or to B7-1 and prevent interaction between the receptor and ligand. Further examples of immune checkpoint inhibitors can be found, for example, in WO2014/144885. Such immune checkpoint inhibitors are incorporated by reference herein. In some aspects of any one of the methods, compositions or kits provided, the immune checkpoint inhibitor is a small molecule inhibitor of an immune checkpoint pathway. In some aspects of any one of the methods, compositions or kits provided, the immune checkpoint inhibitor is a polypeptide that inhibits an immune checkpoint pathway. In some aspects of any one of the methods, compositions or kits provided, the inhibitor is a fusion protein. In some aspects of any one of the methods, compositions or kits provided, the immune checkpoint inhibitor is an antibody. In some aspects of any one of the methods, compositions or kits provided, the antibody is a monoclonal antibody.

Non-limiting examples of immune checkpoint inhibitors include fully human monoclonal antibodies, such as RG7446, BMS-936558/MDX-1106, BMS-936559 (anti-PDL1 antibody), Yervoy/ipilimumab (anti-CTLA-4 checkpoint inhibitor), and Tremelimumab (CTLA-4 blocking antibody); humanized antibodies, such as pidilizumab (CT-011, CureTech Ltd.) and lambrolizumab (MK-3475, Merck, PD-1 blocker); and fusion proteins, such as AMP-224 (Merck). Other examples of checkpoint inhibitors include anti-OX40, PD-L1 monoclonal Antibody (Anti-B7-H1; MEDI4736), Nivolumab (BMS-936558, Bristol-Myers Squibb, anti-PD1 antibody), CT-011 (anti-PD1 antibody), BY55 monoclonal antibody, MPLDL3280A (anti-PDL1 antibody), and MSB0010718C (anti-PDL1 antibody), MDX-1105 (Medarex), MPDL3280A (Genentech), Anti-KIR antibodies such as lirlumab (Innate Pharma) and IPH2101 (Innate Pharma) may perform similar functions in NK cells. Further examples of checkpoint inhibitors include agonistic anti-4-lbb antibody; agonistic anti-CD27 antibody; agonistic anti-GTIR antibody; agonistic anti-OX40 antibody; and antagonistic anti-TIM3 antibody.

### 2. Additional Immunotherapies and Agents

In some aspects, the method further comprises administration of an immunotherapy or an additional agent described herein. In some aspects, the additional agent is an immunostimulator. The term “immunostimulator” as used herein refers to a compound that can stimulate an immune response in a subject, and may include an adjuvant. In some aspects, an immunostimulator is an agent that does not constitute a specific antigen, but can boost the strength and longevity of an immune response to an antigen. Such immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherihia coli, Salmonella minnesota, Salmonella typhimurium, or Shigella flexneri or specifically with MPL® (ASO4), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalene+MPL.), liposomes and liposomal formulations such as ASO1, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments.

In some aspects, the additional agent comprises an agonist for pattern recognition receptors (PRR), including, but not limited to Toll-Like Receptors (TLRs), specifically TLRs 2, 3, 4, 5, 7, 8, 9 and/or combinations thereof. In some aspects, additional agents comprise agonists for Toll-Like Receptors 3, agonists for Toll-Like Receptors 7 and 8, or agonists for Toll-Like Receptor 9; preferably the recited immunostimulators comprise imidazoquinolines; such as R848; adenine derivatives, such as those disclosed in U.S. Pat. No. 6,329,381, U.S. Published Patent Application 2010/0075995, or WO 2010/018132; immunostimulatory DNA; or immunostimulatory RNA. In some aspects, the additional agents also may comprise immunostimulatory RNA molecules, such as but not limited to dsRNA, poly I:C or poly I:poly C12U (available as Ampligen®, both poly I:C and poly I:polyC12U being known as TLR3 stimulants), and/or those disclosed in F. Heil et al., “Species-Specific Recognition of Single-Stranded RNA via Toll-like Receptor 7 and 8” Science 303(5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US 2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. In some aspects, an additional agent may be a TLR-4 agonist, such as bacterial lipopolysaccharide (LPS), VSV-G, and/or HMGB-1. In some aspects, additional agents may comprise TLR-5 agonists, such as flagellin, or portions or derivatives thereof, including but not limited to those disclosed in U.S. Pat. Nos. 6,130,082, 6,585,980, and 7,192,725.

In some aspects, additional agents may be proinflammatory stimuli released from necrotic cells (e.g., urate crystals). In some aspects, additional agents may be activated components of the complement cascade (e.g., CD21, CD35, etc.). In some aspects, additional agents may be activated components of immune complexes. Additional agents also include complement receptor agonists, such as a molecule that binds to CD21 or CD35. In some aspects, the complement receptor agonist induces endogenous complement opsonization of the synthetic nanocarrier. In some aspects, immunostimulators are cytokines, which are small proteins or biological factors (in the range of 5 kD-20 kD) that are released by cells and have specific effects on cell-cell interaction, communication and behavior of other cells. In some aspects, the cytokine receptor agonist is a small molecule, antibody, fusion protein, or aptamer.

In some aspects, the additional agent is a chimeric antigen receptor (CAR). CARs are artificial T cell receptors which graft a specificity onto an immune effector cell. The most common form of these molecules are fusions of single-chain variable fragments (scFv) derived from monoclonal antibodies, fused to CD3-zeta transmembrane and endodomain. Such molecules result in the transmission of a zeta signal in response to recognition by the scFv of its target. An example of such a construct is 14g2a-Zeta, which is a fusion of a scFv derived from hybridoma 14g2a (which recognizes disialoganglioside GD2). When T cells express this molecule (usually achieved by oncoretroviral vector transduction), they recognize and kill target cells that express GD2 (e.g. neuroblastoma cells). The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows the scFv to orient in different directions to enable antigen binding. The transmembrane domain is a typical hydrophobic alpha helix usually derived from the original molecule of the signalling endodomain which protrudes into the cell and transmits the desired signal.

Additional agents that can act as immunostimulators include STING agonists. The STING pathway is a pathway that is involved in the detection of cytosolic DNA. Stimulator of interferon genes (STING), also known as transmembrane protein 173 (TMEM173) and MPYS/MITA/ERIS, is a protein that in humans is encoded by the TMEM173 gene. STING plays an important role in innate immunity. STING induces type I interferon production when cells are infected with intracellular pathogens, such as viruses, mycobacteria and intracellular parasites. Type I interferon, mediated by STING, protects infected cells and nearby cells from local infection in an autocrine and paracrine manner.

STING is encoded by the TMEM173 gene. It works as both a direct cytosolic DNA sensor (CDS) and an adaptor protein in Type I interferon signaling through different molecular mechanisms. It has been shown to activate downstream transcription factors STAT6 and IRF3 through TBK1, which are responsible for antiviral response and innate immune response against intracellular pathogen.

STING resides in the endoplasmic reticulum, but in the presence of cytosolic DNA, the sensor cGAS binds to the DNA and forms cyclic dinucleotides. This di-nucleotide binds to STING and promotes its aggregation and translocation from the ER through the Golgi to perinuclear sites. There, STING complexes with TBK1 and promotes its phosphorylation. Once TBK1 is phosphorylated, it phosphorylates the transcription factor IRF3 that dimerices and traslocates to the nucleus, where it activates the transcription of type I IFN and other innate immune genes.

STING agonsists can include 3′3′-cGAMP fluorinated, fluorinated cyclic diadenylate monophosphate, ZDHHC1, 2′3′-c-di-AM(PS)2 (Rp,Rp), 2′2′-cGAMP, c-di-IMP, 2′3′-cGAM(PS)2 (Rp/Sp), 3′3′-cGAMP, DMXAA, 2′3′-cGAMP, c-di-GMP, c-di-GMP, 2′3′-c-di-GMP, 2′3′-c-di-AMP, c-di-GMP Fluorinated, and c-di-AMP.

In some aspects, the immunotherapy includes cytolytic viral therapy, such administration of an onocolytic virus or modified version thereof. Oncolytic viruses include oncolytic herpes simplex virus, adenovirus, reovirus, measles, Newcastle disease virus, and vaccinia virus.

### 3. Vaccine Immunotherapies

The methods of the disclosure may also include the administration of vaccines. As used herein, the term in vitro administration refers to manipulations performed on cells removed from or outside of a subject, including, but not limited to cells in culture. The term ex vivo administration refers to cells which have been manipulated in vitro, and are subsequently administered to a subject. The term in vivo administration includes all manipulations performed within a subject, including administrations.

In certain aspects of the present disclosure, the compositions may be administered either in vitro, ex vivo, or in vivo. In certain in vitro aspects, autologous T cells are incubated with compositions of this disclosure. The cells can then be used for in vitro analysis, or alternatively for ex vivo administration.

Method aspects of the disclosure include vaccinating a subject with a variety of different immunotherapeutic compositions. In some aspects, the methods further comprise administration of immune cells to the subject. In some aspects, the immune cells are autologous. In some aspects, the immune cells has been contacted with an antigen. In some aspects, the antigen is an antigen expressed by the subject's cancer cells. In some aspects, the antigen is cell free. The term “cell free” refers to a composition that does not have any cellular components. In some aspects, the antigen is an extract from the patient's tumor. In some aspects, the antigen is a polypeptide. In some aspects, the antigen comprises one or more of of tumor cell lysate, apoptotic tumor cell, tumor-associated antigen, and tumor-derived mRNA. In some aspects, the immune cell has been contacted with a maturation agent. In some aspects, the maturation agent is one or more of GM-CSF, IL-1β, TNF-α, and PGE2. In some aspects, the immune cell comprises a chimeric antigen receptor.

In some aspects, the immune cell is an antigen presenting cells. Antigen-presenting cells can be used as a cancer vaccine. Examples of the antigen-presenting cells include dendritic cells, macrophages, B cells, and tumor cells (false antigen-presenting cells) in which a T cell stimulation factor (e.g., B7 or 4-1 BBL) and the like is forcibly expressed by, for example, gene transfer. In some aspects, the antigen presenting cell is a dendritic cell.

The route of administration of the immune cell may be, for example, intratumoral, intracutaneous, subcutaneous, intravenous, intralymphatic, and intraperitoneal administrations. In some aspects, the administration is intratumoral or intrapymphatic. In some aspects, the immune cells are administered directly into a cancer tissue or a lymph node.

In some aspects, the immune cell is a T cell. T cells can also be used as a cancer vaccine. The T cells may be ones that have been contacted with an antigen or with antigen-presenting cells. For example, APCs may be cultured with tumor antigen specific to the patient's cancer to differentiate them, into, for example, CD8-positive cytotoxic T lymphocytes (CTLs) or CD4-positive helper T cells. The T cells thus established may be administered to an individual with cancer.

The origin of the naive T cells is not specifically limited and it may be derived from, for example, peripheral blood of a vertebrate animal. The naive T cell used may be CD8-positive cells or CD4-positive cells isolated from a PBMC fraction. In some aspects, the naive T cells are CD8-positive cells or CD4-positive cells mixed with other cells and components without being isolated from the PBMC fraction in terms of the efficiency of inducing CTLs. For example, when cells of a PBMC fraction are cultured in a medium supplemented with serum and tumor antigen, the PBMCs differentiate into dendritic cell precursors. The dendritic cell precursors then bind to the peptide and differentiate into dendritic cells as the antigen-presenting cells presenting this peptide/tumor antigen. The antigen-presenting cells stimulate the CD8-positive T cells in the PBMCs to differentiate them into CTLs. Thus, the CTLs capable of recognizing the added peptide can be obtained. The CTLs thus obtained may be isolated and used as the cancer vaccine as they are. Alternatively, they may be cultured further in the presence of interleukin such as IL-2, the antigen-presenting cell, and tumor antigen before used as the cancer vaccine. The route of their administration is not specifically limited and examples include intracutaneous, subcutaneous, intravenous, and intratumoral administrations.

In further aspects, the immunotherapy comprises ex vivo administration of dendritic cells, such as dendritic cells that have been contacted with antigens, such as autologous or allogeneic tumor lysate pulsed DCs, DC/tumor cell fusion productions, mRNA transduced DCs and virus-transduced DCs.

### 4. Tumor Cell Vaccines

Melanoma tumor cells may also be used as immunogens using a range of vaccination regimes. Tumor cell vaccines can be designed either as whole melanoma cells from fresh or cryopreserved tumor samples irradiated prior to treatment to halt propagation in the recipient or derived from subcellular components of melanoma cell lysates. Vaccines can either be derived from autologous or allogeneic tumor cells.

Tumar cell vaccines may be combined with other nonspecific adjuvants such as Bacillus Calmette-Gudrin (BCG) or proinflammatory cytokines, such as GM-CSF. In other aspects, autologous tumor cells may be conjugated to haptens such as 2,4-dinitrophenol (DNP; e.g., M-Vax). Allogeneic tumor cell vaccines can be prepared from multiple cell lines and are not derived from the recipient's own cells. This allows for manipulation of tumor cells to express a range of tumor-associated antigens that may induce a wide range of immune responses. Allogeneic tumor cell vaccines are also easier to prepare, standardize and produce, and may have wider clinical applicability. Exemplary allogenic tumor cell vaccines useful as an immunotherapy according to the methods of the disclosure include Canvaxin™ (CancerVax Corp, CA, USA) and Melacine® (Corixa-Montana, MT, USA), which may be used alone or with other agents, such as adjuvants, for example.

In some aspects, the vaccine comprises a peptide vaccine. Numerous melanoma antigens have been identified, and a variety of vaccination strategies have been examined aimed at activating immune responses to recognize and destroy melanoma cells expressing these antigens using vaccines that can direct immune responses against a single HLA-restricted antigen (univalent) or polyvalent vaccines, using multiple antigens or antigenic epitopes. Polyvalent vaccines may increase the probability of eradicating tumors by: circumventing antigenic heterogeneity and loss of antigen expression by cancer cells in progressing tumors; and overcoming HLA restriction.

For antigenic vaccine approaches to therapy, selection of immunodominant MHC-presented epitopes of known tumor-associated antigens is aimed at generating CTL responses against tumor cells expressing these antigens. Antigenic peptides are generally derived from one or more melanoma-associated antigens, such as tyrosinase, tyrosinase-related proteins (TRP-1 and TRP-2), melanoma-associated glycoprotein antigen family (gp100/pmel17) and MART/Melan-A, and also cancer-testis antigens such as NY-ESO-1, melanoma antigen E (MAGE) and B melanoma antigen. Various approaches have aimed to enhance the immunogenic capacity of peptide vaccines by administering these in combination with cytokines (e.g., IL-2, IFN-α2b and GM-CSF), Toll-like receptor (TLR) agonists (e.g., CpG oligodeoxynucleotides and imiquimod) or emulsified with adjuvants (e.g., incomplete Freud's adjuvant, ASO2B and Alum).

In some aspects, the vaccine is a DNA or a viral vaccine. Nucleic acid vaccines, either as naked plasmid DNA or as recombinant attenuated viruses or viral vectors (e.g., retroviruses, adenoviruses, poxviruses and alphaviruses), encode one or more specific epitopes of one or more tumor-associated antigens (e.g., tyrosinase and gp100) that can be recognized by cytotoxic CD8+ T cells. Vaccination administered by intramuscular or intradermal injections should trigger nucleic acid uptake by somatic cells such as keratinocytes or myocytes or by APCs such as DCs with subsequent antigen expression at the site of inoculation. APCs, either directly inoculated or through release of antigen by somatic cells (cross-priming) can then become activated to present antigens to T cells either in situ or upon migration to lymph nodes leading to T-cell maturation and expansion.

### 5. Cytokines

Exemplary cytokine treatments include decarbazine, INF-α2β, IL-2, high-dose IL-2, pegylated IFN-α2β, IFN-α, IFN-7, GM-CSF and IL-2, IL-4, IL-6, IL-12, IL-18 and IL-21.

C. MAPK inhibitors

In some aspects, the compositions comprise a MAPK inhibitor. MAPK inhibitors include those that inhibit MAPK/ERK pathway. Exemplary MAPK inhibitors include vemurafenib, dabrafenib, trametinib, cobimetinib, selumetinib, and combinations thereof. Specific combinations include 1) dabrafenib and cobimetinib and 2) vemurafenib and trametinib. In some aspects, the MAPK inhibitor is a MEK inhibitor. MEK inhibitors include cobimetinib, CI-1040, PD035901, Binimetinib (MEK162), selumetinib, and Trametinib(GSK1120212). In some aspects, the MAPK inihibitor is a Raf inhibitor. Raf inhibitors include, for example, SB590885, PLX4720, XL281, RAF265, encorafenib, dabrafenib, vemurafenib. In some aspects, the Raf inhibitor is an inhibitor of B-Raf. Exemplary B-Raf inhibitors include sorafenib, PLX4032, regorafenib (BAY 73-4506), NVP-BHG712, vemurafenib, and dabarefenib.

Further examples include VX-702 (Vertex), Pamapimod (Roche Pharmaceuticals), Iosmapimod (GW856553; GlaxoSmithKline), Dilmapimod (SB681323; GlaxoSmithKline), Doramapimod (BIRB 796; Boehringer Ingelheim Pharmaceutical), BMS-582949 (Bristol-Myers Squibb), ARRY-797 (Array BioPharma), PH797804 (Pfizer), PF-3644022 (Pfizer), MSC2032964A (Merck Serono), CI-1040 (PD184352; Pfizer), PD0325901 (Pfizer), Selumetinib (AZD6244; Array BioPharma/AstraZeneca), Trametinib (GSK1120212; GlaxoSmithKline), ARRY-438162 (Array BioPharma), ralimetinib, SB203580, and SCIO-469 (Scios).

### D. Additional Agents

In some aspects, the methods and compositions of the disclosure comprises the administration of an additional agent or includes an additional agent in a therapeutic composition. In some aspects, the additional agent is a VEGF-targeted agent. Targeting the tumor vascular microenvironment and preventing growth of metastases by inhibiting new blood vessel formation and supply of vital nutrients may help restrict tumor growth and progression. Melanoma metastases have a prominent vascular component and tumor-induced sentinel lymph-node lymphangiogenesis promotes melanoma metastasis to distant sites, lending merits to anti-angiogenic therapies. In some aspects, the additional agent is a neutralizing or inhibitor antibody directed to VEGF-A, VEGFR, and/or VEGFR-2. One exemplary additional agent that is a VEGF-targeting agent is the bevacizumab (Avastin®, Genentech/Roche; San Francisco, CA, USA). The antibody recognizes an epitope expressed on all VEGF-A isoforms with high affinity and blocks VEGF interaction with both receptors.

In some aspects, the additional agent comprises an antibody that targets Tregs. Tregs are thought to suppress antitumor responses in vivo and may, in part, be responsible for the limited efficacy of strategies aimed at boosting immunity, such as IL-2 and tumor vaccines. An exemplary agent in this category is a CD25 antibody. In some aspects, the CD25 antibody comprises daclizumab.

In some aspects, the additional agent is an agent that targets costimulatory molecules. Other strategies entail activating T cells with agonist mAbs such as those against costimulatory cell surface molecules OX40 and CD137. OX40, expressed on antigen-primed T cells, recognizes its cognate ligand on APCs (DCs, activated B cells and macrophages) mediating the survival and activation of T cells. CD137, also known as 4-1BB, a membrane glycoprotein belonging to the tumor necrosis factor receptor family is expressed on primed T cells and other immune cells (e.g., NKs, monocytes, macrophages, neutrophils, mast cells and DCs). CD137 recognizes a ligand on the surface of APCs and this interaction is thought to induce T-cell proliferation and maturation. Agonistic antibodies to CD137 have been shown to induce antitumoral immune responses associated with increased T-cell activation and infiltration in tumor lesions.

In some aspects, the additional agent is an anti-CD40 antibody. CD40 is expressed on solid tumors including melanoma. CD40 represents a potential therapeutic target in that activation of CD40 promotes apoptosis within tumor cells. It is also responsible in part for the generation of tumor-specific T-cell responses, as CD40L is expressed on the surface of activated T lymphocytes. CD40-CD40L interaction on T lymphocytes mediates increased immune stimulation and cytotoxicity. CD40 stimulation is also thought to allow for DC maturation, a process which is inhibited within the tumor microenvironment and is thought to be contributory to immune escape.

In some aspects, the additional agent comprises an agent that targets integran or fibronectin isoforms. In some aspects, the agent targets integrins of the αv family that are involved in tumor-associated angiogenesis. Exemplary agents include antibodies such as the chimeric volociximab (M200) against α5β1 integrin, the humanized mAb etaracizumab (Abegrin™ [MedImmune Inc., MD, USA], Vitaxin or MEDI-522) recognising the integrin αvβ3, and the human antibody CNTO 95 against av integrin.

In some aspects, the agent targets a splice variant of fibronectin, such as the isoform extra domain-B (ED-B) fibronectin, a protein found in the subendothelial extracellular matrix in tumor lesions that is produced by melanoma cells and thought to promote tumor growth and angiogenesis. In some aspects, the agent is an antibody that recognizes the ED-B fibronectin. In a specific aspect, the agent is an antibody recognizing ED-B fibronectin fused with the human pluripotent cytokine IL-12 (e.g. AS1409—Antisoma; London, UK).

In some aspects, the additional agent comprises a bisphosphonate. In some aspects, the biphoshpnate is used in combination with IL-2. An exemplary biphosphate comprises zoledronate.

In some aspects, the additional agent comprises a chemotherapeutic agent. Chemotherapies include, for example, cisplatin (CDDP), carboplatin, dacarbazine, temozolomide, nab-paclitaxel, paclitaxel, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabine, navelbine, farnesyl-protein transferase inhibitors, transplatinum, 5-fluorouracil, vincristin, vinblastin and methotrexate, or any analog or derivative variant of the foregoing.

In some aspects, the chemotherapeutic agent is selected from dacarbazine, temozolomide, nab-paclitaxel, paclitaxel, cisplatin, carboplatin, and vinblastine.

Suitable therapeutic agents include, for example, vinca alkaloids, agents that disrupt microtubule formation (such as colchicines and its derivatives), anti-angiogenic agents, therapeutic antibodies, tyrosine kinase targeting agent (such as tyrosine kinase inhibitors), serine kinase targeting agents, transitional metal complexes, proteasome inhibitors, antimetabolites (such as nucleoside analogs), alkylating agents, platinum-based agents, anthracycline antibiotics, topoisomerase inhibitors, macrolides, therapeutic antibodies, retinoids (such as all-trans retinoic acids or a derivatives thereof); geldanamycin or a derivative thereof (such as 17-AAG), and other standard chemotherapeutic agents well recognized in the art.

### E. Inhibitory Antibodies

In certain aspects, an antibody or a fragment thereof that binds to at least a portion of a B-Raf or MEK protein and inhibits the protein's activity and/or function is used in the methods and compositions described herein.

In some aspects, the antibody is a monoclonal antibody or a polyclonal antibody. In some aspects, the antibody is a chimeric antibody, an affinity matured antibody, a humanized antibody, or a human antibody. In some aspects, the antibody is an antibody fragment. In some aspects, the antibody is a Fab, Fab′, Fab′-SH, F(ab′)2, or scFv. In one aspect, the antibody is a chimeric antibody, for example, an antibody comprising antigen binding sequences from a non-human donor grafted to a heterologous non-human, human or humanized sequence (e.g., framework and/or constant domain sequences). In one aspect, the non-human donor is a mouse. In one aspect, an antigen binding sequence is synthetic, e.g., obtained by mutagenesis (e.g., phage display screening, etc.). In one aspect, a chimeric antibody has murine V regions and human C region. In one aspect, the murine light chain V region is fused to a human kappa light chain or a human IgG1 C region.

Examples of antibody fragments include, without limitation: (i) the Fab fragment, consisting of VL, VH, CL and CHi domains; (ii) the “Fd” fragment consisting of the VH and CHi domains; (iii) the “Fv” fragment consisting of the VL and VH domains of a single antibody; (iv) the “dAb” fragment, which consists of a VH domain; (v) isolated CDR regions; (vi) F(ab′)2 fragments, a bivalent fragment comprising two linked Fab fragments; (vii) single chain Fv molecules (“scFv”), wherein a VH domain and a VL domain are linked by a peptide linker which allows the two domains to associate to form a binding domain; (viii) bi-specific single chain Fv dimers (see U.S. Pat. No. 5,091,513) and (ix) diabodies, multivalent or multispecific fragments constructed by gene fusion (U.S. Patent Pub. 2005/0214860). Fv, scFv or diabody molecules may be stabilized by the incorporation of disulphide bridges linking the VH and VL domains. Minibodies comprising a scFv joined to a CH3 domain may also be made (Hu et al, 1996).

A monoclonal antibody is a single species of antibody wherein every antibody molecule recognizes the same epitope because all antibody producing cells are derived from a single B-lymphocyte cell line. Hybridoma technology involves the fusion of a single B lymphocyte from a mouse previously immunized with an antigen with an immortal myeloma cell (usually mouse myeloma). This technology provides a method to propagate a single antibody-producing cell for an indefinite number of generations, such that unlimited quantities of structurally identical antibodies having the same antigen or epitope specificity (monoclonal antibodies) may be produced. However, in therapeutic applications a goal of hybridoma technology is to reduce the immune reaction in humans that may result from administration of monoclonal antibodies generated by the non-human (e.g., mouse) hybridoma cell line.

Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent and human amino acid sequences. In “humanized” monoclonal antibodies, only the hypervariable CDR is derived from mouse monoclonal antibodies, and the framework regions are derived from human amino acid sequences. It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

It is possible to create engineered antibodies, using monoclonal and other antibodies and recombinant DNA technology to produce other antibodies or chimeric molecules which retain the antigen or epitope specificity of the original antibody, i.e., the molecule has a binding domain. Such techniques may involve introducing DNA encoding the immunoglobulin variable region or the CDRs of an antibody to the genetic material for the framework regions, constant regions, or constant regions plus framework regions, of a different antibody. See, for instance, U.S. Pat. Nos. 5,091,513, and 6,881,557, which are incorporated herein by this reference.

By known means as described herein, polyclonal or monoclonal antibodies, binding fragments and binding domains and CDRs (including engineered forms of any of the foregoing), may be created that are specific to a protein described herein, one or more of its respective epitopes, or conjugates of any of the foregoing, whether such antigens or epitopes are isolated from natural sources or are synthetic derivatives or variants of the natural compounds.

Antibodies may be produced from any animal source, including birds and mammals. Particularly, the antibodies may be ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by this reference. These techniques are further described in: Marks (1992); Stemmer (1994); Gram et al. (1992); Barbas et al. (1994); and Schier et al. (1996).

Methods for producing polyclonal antibodies in various animal species, as well as for producing monoclonal antibodies of various types, including humanized, chimeric, and fully human, are well known in the art. Methods for producing these antibodies are also well known. For example, the following U.S. patents and patent publications provide enabling descriptions of such methods and are herein incorporated by reference: U.S. Patent publication Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024. All patents, patent publications, and other publications cited herein and therein are hereby incorporated by reference in the present application.

It is fully expected that antibodies to B-Raf or MEK will have the ability to neutralize or counteract the effects of the protein regardless of the animal species, monoclonal cell line or other source of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into binding fragments having the binding domain or CDR. Removal of the Fc portion reduces the likelihood that the antigen binding fragment will elicit an undesirable immunological response and, thus, antibodies without Fc may be particularly useful for prophylactic or therapeutic treatments. As described above, antibodies may also be constructed so as to be chimeric, partially or fully human, so as to reduce or eliminate the adverse immunological consequences resulting from administering to an animal an antibody that has been produced in, or has sequences from, other species.

In some aspects, the inhibitor is a peptide, polypeptide, or protein inhibitor. In some aspects, the inhibitor is an antagonistic antibody.

### F. Nucleic Acid Inhibitors

Inhibitory nucleic acids or any ways of inhibiting gene expression of BRAF and MEK known in the art are contemplated in certain aspects. Examples of an inhibitory nucleic acid include but are not limited to siRNA (small interfering RNA), short hairpin RNA (shRNA), double-stranded RNA, an antisense oligonucleotide, a ribozyme, and a nucleic acid encoding thereof. An inhibitory nucleic acid may inhibit the transcription of a gene or prevent the translation of a gene transcript in a cell. An inhibitory nucleic acid may be from 16 to 1000 nucleotides long, and in certain aspects from 18 to 100 nucleotides long. The nucleic acid may have nucleotides of at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, 90 or any range derivable therefrom.

As used herein, “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

In some aspects, the nucleic acid inhibitor is comprises a modification, such as a chemical modification or a modified base. In some aspects, one or more (e.g., 1, 2, 3, 4, 5, 6, 7, 8, 9, 10, 11, 12, 13, 14, 15, 16, 17, 18, 19. 20, 21, 22, 23, 24, 25, 26, 27, 28, 29, 30, 31, 32, 33, 34, 35, 36, 37, 38, 39, or 40 (or any derivable range therein) of the nucleotide positions in one or both strands of an siRNA molecule are modified. Modifications include nucleic acid sugar modifications, base modifications, backbone (internucleotide linkage) modifications, non-nucleotide modifications, and/or any combination thereof. In certain instances, purine and pyrimidine nucleotides are differentially modified. For example, purine and pyrimidine nucleotides can be differentially modified at the 2′-sugar position (i.e., at least one purine has a different modification from at least one pyrimidine in the same or different strand at the 2′-sugar position). In other instances, at least one modified nucleotide is a 2′-deoxy-2′-fluoro nucleotide, a 2′-deoxy nucleotide, or a 2′-O-alkyl nucleotide. In certain aspects, the siRNA molecule has 3′ overhangs of one, two, three, or four nucleotide(s) on one or both of the strands. In other aspects, the siRNA lacks overhangs (i.e., has blunt ends). The overhangs can be modified or unmodified. Examples of modified nucleotides in the overhangs include, but are not limited to, 2′-O-alkyl nucleotides, 2′-deoxy-2′-fluoro nucleotides, or 2′-deoxy nucleotides. The overhang nucleotides in the antisense strand can comprise nucleotides that are complementary to nucleotides in the Bach1 target sequence. Likewise, the overhangs in the sense stand can comprise nucleotides that are in the Bach1 target sequence. In certain instances, the siRNA molecules have two 3′ overhang nucleotides on the antisense stand that are 2′-O-alkyl nucleotides and two 3′ overhang nucleotides on the sense stand that are 2′-deoxy nucleotides.

Particularly, an inhibitory nucleic acid may be capable of decreasing the expression of a protein or mRNA by at least 10%, 20%, 30%, or 40%, more particularly by at least 50%, 60%, or 70%, and most particularly by at least 75%, 80%, 90%, 95% or more or any range or value in between the foregoing.

In further aspects, there are synthetic nucleic acids that are MAPK inhibitors. An inhibitor may be between 17 to 25 nucleotides in length and comprises a 5′ to 3′ sequence that is at least 90% complementary to the 5′ to 3′ sequence of a mature BACH1 mRNA. In certain aspects, an inhibitor molecule is 17, 18, 19, 20, 21, 22, 23, 24, or 25 nucleotides in length, or any range derivable therein. Moreover, an inhibitor molecule has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature MAPK gene (e.g. BRAF or MEK) mRNA, particularly a mature, naturally occurring mRNA. One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

### G. Combination Therapies

The methods and compositions may include chemotherapy, therapeutic agents, surgical removal of cancerous cells, radiation therapy, and combinations thereof. In some aspects, the treatment regimen excludes one or more of chemotherapy, therapeutic agents, surgical removal of cancerous cells and/or radiation therapy.

In some aspects, the treatment regimen comprises a combination of the one or more chemotherapeutic agents, therapeutic agents, inhibitors, and/or immunotherapies described herein. In some aspects, the treatment regimen excludes one or more of the chemotherapeutic agents, therapeutic agents, inhibitors, and/or immunotherapies described herein.

In further aspects a combination of therapeutic treatment agents is administered to cancer cells. The therapeutic agents may be administered serially (within minutes, hours, or days of each other) or in parallel; they also may be administered to the patient in a pre-mixed single composition.

Various combinations of more than an anticancer modality, agent or compound (or a combination of such agents and/or compounds) may be employed, for example, a first anticancer modality, agent or compound is “A” and a second anticancer modality, agent or compound (or a combination of such modalities, agents and/or compounds) given as part of an anticancer therapy regime, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the therapeutic compounds or agents to a patient will follow general protocols for the administration of such compounds, taking into account the toxicity, if any, of the therapy. It is expected that the treatment cycles would be repeated as necessary. It also is contemplated that various standard therapies, as well as surgical intervention, may be applied in combination with the described therapy.

Radiation therapy that cause DNA damage and have been used extensively include what are commonly known as 7-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Alternative cancer therapy include any cancer therapy other than surgery, chemotherapy and radiation therapy, such as immunotherapy, gene therapy, hormonal therapy or a combination thereof. Subjects identified with poor prognosis using the present methods may not have favorable response to conventional treatment(s) alone and may be prescribed or administered one or more alternative cancer therapy per se or in combination with one or more conventional treatments.

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

### III. Methods of Treatment

### A. Treatment of Cancer

Certain aspects are directed to methods of treating cancer, such as skin cancer, based on certain parameters such as biomarker levels and cancer phenotypes. Any known treatments that are contemplated for treating a cancer or skin cancer can be used.

In certain aspects, there may be provided methods for treating a subject determined to have cancer and with a predetermined expression profile of one or more biomarkers disclosed herein.

In a further aspect, biomarkers and related systems that can establish a prognosis of cancer patients can be used to identify patients who may get benefit of conventional single or combined modality therapy. In the same way, those patients who do not get much benefit from such conventional single or combined modality therapy can be identified and can be offered alternative treatment(s).

Approximately 60% of persons with cancer will undergo surgery of some type, which includes preventative, diagnostic or staging, curative and palliative surgery. Curative surgery is a cancer treatment that may be used in conjunction with other therapies, such as the treatment, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the treatment methods described herein may be used in conjunction with removal of superficial cancers, precancers, or incidental amounts of normal tissue.

In some aspects, the methods may further comprise a therapy described herein such as those described below.

Laser therapy is the use of high-intensity light to destroy tumor cells. Laser therapy affects the cells only in the treated area. Laser therapy may be used to destroy cancerous tissue and relieve a blockage in the esophagus when the cancer cannot be removed by surgery. The relief of a blockage can help to reduce symptoms, especially swallowing problems.

Photodynamic therapy (PDT), a type of laser therapy, involves the use of drugs that are absorbed by cancer cells; when exposed to a special light, the drugs become active and destroy the cancer cells. PDT may be used to relieve symptoms of esophageal cancer such as difficulty swallowing.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well. A patient may be administered a single compound or a combination of compounds described herein in an amount that is, is at least, or is at most 0.1, 0.2, 0.3, 0.4, 0.5, 0.6, 0.7, 0.8, 0.9, 1, 2,3,4,5, 6,7, 8,9, 10, 11, 12, 13, 14, 15, 16, 17, 18, 19, 20, 21, 22,23,24,25,26,27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg (or any range derivable therein). A patient may be administered a single compound or a combination of compounds described herein in an amount that is, is at least, or is at most 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500 mg/kg/day (or any range derivable therein).

The cancers amenable for treatment include skin cancers of various types, locations, sizes, and characteristics. In some aspects, the skin cancer is de-differentiated melanoma or amelanotic melanoma.

### B. ROC Analysis

In statistics, a receiver operating characteristic (ROC), or ROC curve, is a graphical plot that illustrates the performance of a binary classifier system as its discrimination threshold is varied. The curve is created by plotting the true positive rate against the false positive rate at various threshold settings. (The true-positive rate is also known as sensitivity in biomedical informatics, or recall in machine learning. The false-positive rate is also known as the fall-out and can be calculated as 1−specificity). The ROC curve is thus the sensitivity as a function of fall-out. In general, if the probability distributions for both detection and false alarm are known, the ROC curve can be generated by plotting the cumulative distribution function (area under the probability distribution from −infinity to +infinity) of the detection probability in the y-axis versus the cumulative distribution function of the false-alarm probability in x-axis.

ROC analysis provides tools to select possibly optimal models and to discard suboptimal ones independently from (and prior to specifying) the cost context or the class distribution. ROC analysis is related in a direct and natural way to cost/benefit analysis of diagnostic decision making.

The ROC curve was first developed by electrical engineers and radar engineers during World War II for detecting enemy objects in battlefields and was soon introduced to psychology to account for perceptual detection of stimuli. ROC analysis since then has been used in medicine, radiology, biometrics, and other areas for many decades and is increasingly used in machine learning and data mining research.

The ROC is also known as a relative operating characteristic curve, because it is a comparison of two operating characteristics (TPR and FPR) as the criterion changes. ROC analysis curves are known in the art and described in Metz C E (1978) Basic principles of ROC analysis. Seminars in Nuclear Medicine 8:283-298; Youden W J (1950) An index for rating diagnostic tests. Cancer 3:32-35; Zweig M H, Campbell G (1993) Receiver-operating characteristic (ROC) plots: a fundamental evaluation tool in clinical medicine. Clinical Chemistry 39:561-577; and Greiner M, Pfeiffer D, Smith R D (2000) Principles and practical application of the receiver-operating characteristic analysis for diagnostic tests. Preventive Veterinary Medicine 45:23-41, which are herein incorporated by reference in their entirety.

ROC analysis is useful for determining cut-off values for expression levels, protein levels, or activity levels. Such cut-off values can be used to determine a patient's prognosis and to predict a patient's response to a particular therapy.

### C. Biological Sample Preparation

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain aspects the sample is obtained from a biopsy from skin tissue by any of the biopsy methods previously mentioned. In other aspects the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects the sample is obtained from melanocytes or skin cells derived from a tumor or neoplasm. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain aspects the samples are obtained by biopsy. In other aspects the sample is obtained by swabbing, scraping, phlebotomy, or any other methods known in the art. In some cases, the sample may be obtained, stored, or transported using components of a kit of the present methods. In some cases, multiple samples, such as multiple cancerous samples may be obtained for diagnosis by the methods described herein. In other cases, multiple samples, such as one or more samples from one tissue type (for example breast) and one or more samples from another tissue may be obtained for diagnosis by the methods. Samples may be obtained at different times are stored and/or analyzed by different methods. For example, a sample may be obtained and analyzed by routine staining methods or any other cytological analysis methods.

In some aspects the biological sample may be obtained by a physician, nurse, or other medical professional such as a medical technician, dermatologist, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, or phlebotomy. The method of needle aspiration may further include fine needle aspiration, core needle biopsy, vacuum assisted biopsy, or large core biopsy. In some aspects, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one aspect, the sample is a fine needle aspirate of a colorectal or a suspected colorectal tumor or neoplasm. In some cases, the fine needle aspirate sampling procedure may be guided by the use of an ultrasound, X-ray, or other imaging device.

In some aspects of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some aspects of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, proteins, polypeptides, genes, gene fragments, expression products, gene expression products, protein expression products or fragments, or gene expression product fragments of an individual to be tested. Methods for determining sample suitability and/or adequacy are provided.

In some aspects, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### IV. Analysis of Gene Expression

A gene shall be understood to be specifically expressed in a certain cell type if the expression level of said gene in said cell type is at least 2-fold, 5-fold, 10-fold, 100-fold, 1000-fold, or 10000-fold higher than in a reference cell type, or in a mixture of reference cell types. Reference cell types include non-cancerous tissue cells or a heterogeneous population of cancers.

Comparison of multiple marker genes with a threshold level can be performed as follows: 1. The individual marker genes are compared to their respective threshold levels. 2. The number of marker genes, the expression level of which is above their respective threshold level, is determined. 3. If a marker genes is expressed above its respective threshold level, then the expression level of the marker gene is taken to be “above the threshold level”.

In certain aspects, the determination of expression levels is on a gene chip, such as an Affymetrix™ gene chip. In another aspect, the determination of expression levels is done by kinetic real time PCR.

In certain aspects, the methods can relate to a system for performing such methods, the system comprising (a) apparatus or device for storing data on the biomarker level of the patient; (b) apparatus or device for determining the expression level of at least one marker gene or activity; (c) apparatus or device for comparing the expression level of the first marker gene or activity with a predetermined first threshold value; (d) apparatus or device for determining the expression level of at least one second, third, fourth, 5^{th}, 6^{th} or more marker gene or activity and for comparing with a corresponding predetermined threshold; and (e) computing apparatus or device programmed to provide a unfavorable or poor prognosis or favorable prognosis based on the comparisons.

The person skilled in the art readily appreciates that an unfavorable or poor prognosis can be given if the expression level of the first marker gene with the predetermined first threshold value indicates a tumor that is likely to recur or not respond well to standard therapies.

The expression patterns can also be compared by using one or more ratios between the expression levels of different cancer biomarkers. Other suitable measures or indicators can also be employed for assessing the relationship or difference between different expression patterns.

The expression levels of cancer biomarkers can be compared to reference expression levels using various methods. These reference levels can be determined using expression levels of a reference based on all cancer patients. Alternatively, it can be based on an internal reference such as a gene that is expressed in all cells. In some aspects, the reference is a gene expressed in cancer cells at a higher level than any biomarker. Any comparison can be performed using the fold change or the absolute difference between the expression levels to be compared. One or more cancer biomarkers can be used in the comparison. It is contemplated that 1, 2, 3, 4, 5, 6, 7, 8, 9, 10, and/or 11 biomarkers (or any range derivable therein) may be compared to each other and/or to a reference that is internal or external. A person of ordinary skill in the art would know how to do such comparisons.

Comparisons or results from comparisons may reveal or be expressed as x-fold increase or decrease in expression relative to a standard or relative to another biomarker or relative to the same biomarker but in a different class of prognosis. In some aspects, patients with a poor prognosis have a relatively high level of expression (overexpression) or relatively low level of expression (underexpression) when compared to patients with a better or favorable prognosis, or vice versa.

Fold increases or decreases may be, be at least, or be at most 1-, 2-, 3-, 4-, 5-, 6-, 7-8-, 9-, 10-, 11-, 12-, 13-, 14-, 15-, 16-, 17-, 18-, 19-, 20-, 25-, 30-, 35-, 40-, 45-, 50-, 55-, 60-65-, 70-, 75-, 80-, 85-, 90-, 95-, 100- or more, or any range derivable therein. Alternatively, differences in expression may be expressed as a percent decrease or increase, such as at least or at most 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 1000% difference, or any range derivable therein.

Other ways to express relative expression levels are with normalized or relative numbers such as 0.001, 0.002, 0.003, 0.004, 0.005, 0.006, 0.007, 0.008, 0.009, 0.01, 0.02, 0.03. 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, or any range derivable therein. In some aspects, the levels can be relative to a control.

Algorithms, such as the weighted voting programs, can be used to facilitate the evaluation of biomarker levels. In addition, other clinical evidence can be combined with the biomarker-based test to reduce the risk of false evaluations. Other cytogenetic evaluations may be considered in some aspects.

Any biological sample from the patient that contains cancer cells may be used to evaluate the expression pattern of any biomarker discussed herein. In some aspects, a biological sample from a tumor is used. Evaluation of the sample may involve, though it need not involve, panning (enriching) for cancer cells or isolating the cancer cells.

### A. Measurement of Gene Expression Using Nucleic Acids

Testing methods based on differentially expressed gene products are well known in the art. In accordance with one aspect, the differential expression patterns of cancer biomarkers can be determined by measuring the levels of RNA transcripts of these genes, or genes whose expression is modulated by the these genes, in the patient's cancer cells. Suitable methods for this purpose include, but are not limited to, RT-PCR, Northern Blot, in situ hybridization, Southern Blot, slot-blotting, nuclease protection assay and oligonucleotide arrays.

In certain aspects, RNA isolated from cancer cells can be amplified to cDNA or cRNA before detection and/or quantitation. The isolated RNA can be either total RNA or mRNA. The RNA amplification can be specific or non-specific. Suitable amplification methods include, but are not limited to, reverse transcriptase PCR, isothermal amplification, ligase chain reaction, and Qbeta replicase. The amplified nucleic acid products can be detected and/or quantitated through hybridization to labeled probes. In some aspects, detection may involve fluorescence resonance energy transfer (FRET) or some other kind of quantum dots.

Amplification primers or hybridization probes for a cancer biomarker can be prepared from the gene sequence or obtained through commercial sources, such as Affymatrix. In certain aspects the gene sequence is identical or complementary to at least 8 contiguous nucleotides of the coding sequence.

Sequences suitable for making probes/primers for the detection of their corresponding cancer biomarkers include those that are identical or complementary to all or part of the cancer biomarker genes described herein. These sequences are all nucleic acid sequences of cancer biomarkers.

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In one aspect, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions. In some aspects, because each of the biomarkers has more than one human sequence, it is contemplated that probes and primers may be designed for use with each of these sequences. For example, inosine is a nucleotide frequently used in probes or primers to hybridize to more than one sequence. It is contemplated that probes or primers may have inosine or other design implementations that accommodate recognition of more than one human sequence for a particular biomarker.

For applications requiring high selectivity, one will typically desire to employ relatively high stringency conditions to form the hybrids. For example, relatively low salt and/or high temperature conditions, such as provided by about 0.02 μM to about 0.10 μM NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

In another aspect, the probes/primers for a gene are selected from regions which significantly diverge from the sequences of other genes. Such regions can be determined by checking the probe/primer sequences against a human genome sequence database, such as the Entrez database at the NCBI. One algorithm suitable for this purpose is the BLAST algorithm. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always >0) and N (penalty score for mismatching residues; always <0). The BLAST algorithm parameters W, T, and X determine the sensitivity and speed of the alignment. These parameters can be adjusted for different purposes, as appreciated by one of ordinary skill in the art.

In one aspect, quantitative RT-PCR (such as TaqMan, ABI) is used for detecting and comparing the levels of RNA transcripts in cancer samples. Quantitative RT-PCR involves reverse transcription (RT) of RNA to cDNA followed by relative quantitative PCR (RT-PCR). The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived may be determined for the respective tissues or cells. This direct proportionality between the concentration of the PCR products and the relative mRNA abundances is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable cDNAs may be normalized to some independent standard, which may be based on either internally existing RNA species or externally introduced RNA species. The abundance of a particular mRNA species may also be determined relative to the average abundance of all mRNA species in the sample.

In one aspect, the PCR amplification utilizes one or more internal PCR standards. The internal standard may be an abundant housekeeping gene in the cell or it can specifically be GAPDH, GUSB and β-2 microglobulin. These standards may be used to normalize expression levels so that the expression levels of different gene products can be compared directly. A person of ordinary skill in the art would know how to use an internal standard to normalize expression levels.

A problem inherent in clinical samples is that they are of variable quantity and/or quality. This problem can be overcome if the RT-PCR is performed as a relative quantitative RT-PCR with an internal standard in which the internal standard is an amplifiable cDNA fragment that is similar or larger than the target cDNA fragment and in which the abundance of the mRNA encoding the internal standard is roughly 5-100 fold higher than the mRNA encoding the target. This assay measures relative abundance, not absolute abundance of the respective mRNA species.

In another aspect, the relative quantitative RT-PCR uses an external standard protocol. Under this protocol, the PCR products are sampled in the linear portion of their amplification curves. The number of PCR cycles that are optimal for sampling can be empirically determined for each target cDNA fragment. In addition, the reverse transcriptase products of each RNA population isolated from the various samples can be normalized for equal concentrations of amplifiable cDNAs.

Nucleic acid arrays can also be used to detect and compare the differential expression patterns of cancer biomarkers in cancer cells. The probes suitable for detecting the corresponding cancer biomarkers can be stably attached to known discrete regions on a solid substrate. As used herein, a probe is “stably attached” to a discrete region if the probe maintains its position relative to the discrete region during the hybridization and the subsequent washes. Construction of nucleic acid arrays is well known in the art. Suitable substrates for making polynucleotide arrays include, but are not limited to, membranes, films, plastics and quartz wafers.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. In some aspects, the density may be 50, 100, 200, 300, 400, 500 or more probes/cm².

Specifically contemplated are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of one or more cancer biomarkers with respect to diagnostic, prognostic, and treatment methods.

Certain aspects may involve the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon. Such arrays may vary in a number of different ways, including average probe length, sequence or types of probes, nature of bond between the probe and the array surface, e.g. covalent or non-covalent, and the like. The labeling and screening methods and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some aspects. In certain aspects, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm2. The surface area of the array can be about or less than about 1, 1.6, 2, 3, 4, 5, 6, 7, 8, 9, or 10 cm2.

Moreover, a person of ordinary skill in the art could readily analyze data generated using an array. Such protocols include information found in WO 9743450; WO 03023058; WO 03022421; WO 03029485; WO 03067217; WO 03066906; WO 03076928; WO 03093810; WO 03100448A1, all of which are specifically incorporated by reference.

In one aspect, nuclease protection assays are used to quantify RNAs derived from the cancer samples. There are many different versions of nuclease protection assays known to those practiced in the art. The common characteristic that these nuclease protection assays have is that they involve hybridization of an antisense nucleic acid with the RNA to be quantified. The resulting hybrid double-stranded molecule is then digested with a nuclease that digests single-stranded nucleic acids more efficiently than double-stranded molecules. The amount of antisense nucleic acid that survives digestion is a measure of the amount of the target RNA species to be quantified. An example of a nuclease protection assay that is commercially available is the RNase protection assay manufactured by Ambion, Inc. (Austin, Tex.).

### B. Measurement of Gene Expression Using Proteins and Polypeptides

In other aspects, the differential expression patterns of cancer biomarkers can be determined by measuring the levels of polypeptides encoded by these genes in cancer cells. Methods suitable for this purpose include, but are not limited to, immunoassays such as ELISA, RIA, FACS, dot blot, Western Blot, immunohistochemistry, and antibody-based radioimaging. Protocols for carrying out these immunoassays are well known in the art. Other methods such as 2-dimensional SDS-polyacrylamide gel electrophoresis can also be used. These procedures may be used to recognize any of the polypeptides encoded by the cancer biomarker genes described herein.

One example of a method suitable for detecting the levels of target proteins in peripheral blood samples is ELISA. In an exemplifying ELISA, antibodies capable of binding to the target proteins encoded by one or more cancer biomarker genes are immobilized onto a selected surface exhibiting protein affinity, such as wells in a polystyrene or polyvinylchloride microtiter plate. Then, cancer cell samples to be tested are added to the wells. After binding and washing to remove non-specifically bound immunocomplexes, the bound antigen(s) can be detected. Detection can be achieved by the addition of a second antibody which is specific for the target proteins and is linked to a detectable label. Detection may also be achieved by the addition of a second antibody, followed by the addition of a third antibody that has binding affinity for the second antibody, with the third antibody being linked to a detectable label. Before being added to the microtiter plate, cells in the peripheral blood samples can be lysed using various methods known in the art. Proper extraction procedures can be used to separate the target proteins from potentially interfering substances.

In another ELISA aspect, the cancer cell samples containing the target proteins are immobilized onto the well surface and then contacted with the antibodies. After binding and washing to remove non-specifically bound immunocomplexes, the bound antigen is detected. Where the initial antibodies are linked to a detectable label, the immunocomplexes can be detected directly. The immunocomplexes can also be detected using a second antibody that has binding affinity for the first antibody, with the second antibody being linked to a detectable label.

Another typical ELISA involves the use of antibody competition in the detection. In this ELISA, the target proteins are immobilized on the well surface. The labeled antibodies are added to the well, allowed to bind to the target proteins, and detected by means of their labels. The amount of the target proteins in an unknown sample is then determined by mixing the sample with the labeled antibodies before or during incubation with coated wells. The presence of the target proteins in the unknown sample acts to reduce the amount of antibody available for binding to the well and thus reduces the ultimate signal.

Different ELISA formats can have certain features in common, such as coating, incubating or binding, washing to remove non-specifically bound species, and detecting the bound immunocomplexes. For instance, in coating a plate with either antigen or antibody, the wells of the plate can be incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test samples. Examples of these nonspecific proteins include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means can also be used. After binding of a protein or antibody to the well, coating with a non-reactive material to reduce background, and washing to remove unbound material, the immobilizing surface is contacted with the control and/or clinical or biological sample to be tested under conditions effective to allow immunocomplex (antigen/antibody) formation. These conditions may include, for example, diluting the antigens and antibodies with solutions such as BSA, bovine gamma globulin (BGG) and phosphate buffered saline (PBS)/Tween and incubating the antibodies and antigens at room temperature for about 1 to 4 hours or at 49° C. overnight. Detection of the immunocomplex then requires a labeled secondary binding ligand or antibody, or a secondary binding ligand or antibody in conjunction with a labeled tertiary antibody or third binding ligand.

After all of the incubation steps in an ELISA, the contacted surface can be washed so as to remove non-complexed material. For instance, the surface may be washed with a solution such as PBS/Tween, or borate buffer. Following the formation of specific immunocomplexes between the test sample and the originally bound material, and subsequent washing, the occurrence of the amount of immunocomplexes can be determined.

To provide a detecting means, the second or third antibody can have an associated label to allow detection. In one aspect, the label is an enzyme that generates color development upon incubating with an appropriate chromogenic substrate. Thus, for example, one may contact and incubate the first or second immunocomplex with a urease, glucose oxidase, alkaline phosphatase or hydrogen peroxidase-conjugated antibody for a period of time and under conditions that favor the development of further immunocomplex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl)-benzhiazoline-6-sulfonic acid (ABTS) and hydrogen peroxide, in the case of peroxidase as the enzyme label. Quantitation can be achieved by measuring the degree of color generation, e.g., using a spectrophotometer.

Another suitable method is RIA (radioimmunoassay). An example of RIA is based on the competition between radiolabeled-polypeptides and unlabeled polypeptides for binding to a limited quantity of antibodies. Suitable radiolabels include, but are not limited to, I¹²⁵. In one aspect, a fixed concentration of I¹²⁵-labeled polypeptide is incubated with a series of dilution of an antibody specific to the polypeptide. When the unlabeled polypeptide is added to the system, the amount of the I¹²⁵-polypeptide that binds to the antibody is decreased. A standard curve can therefore be constructed to represent the amount of antibody-bound I¹²⁵-polypeptide as a function of the concentration of the unlabeled polypeptide. From this standard curve, the concentration of the polypeptide in unknown samples can be determined. Various protocols for conducting RIA to measure the levels of polypeptides in cancer cell samples are well known in the art.

Suitable antibodies include, but are not limited to, polyclonal antibodies, monoclonal antibodies, chimeric antibodies, humanized antibodies, single chain antibodies, Fab fragments, and fragments produced by a Fab expression library.

Antibodies can be labeled with one or more detectable moieties to allow for detection of antibody-antigen complexes. The detectable moieties can include compositions detectable by spectroscopic, enzymatic, photochemical, biochemical, bioelectronic, immunochemical, electrical, optical or chemical means. The detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

Protein array technology is discussed in detail in Pandey and Mann (2000) and MacBeath and Schreiber (2000), each of which is herein specifically incorporated by reference. These arrays typically contain thousands of different proteins or antibodies spotted onto glass slides or immobilized in tiny wells and allow one to examine the biochemical activities and binding profiles of a large number of proteins at once. To examine protein interactions with such an array, a labeled protein is incubated with each of the target proteins immobilized on the slide, and then one determines which of the many proteins the labeled molecule binds. In certain aspects such technology can be used to quantitate a number of proteins in a sample, such as a cancer biomarker proteins.

The basic construction of protein chips has some similarities to DNA chips, such as the use of a glass or plastic surface dotted with an array of molecules. These molecules can be DNA or antibodies that are designed to capture proteins. Defined quantities of proteins are immobilized on each spot, while retaining some activity of the protein. With fluorescent markers or other methods of detection revealing the spots that have captured these proteins, protein microarrays are being used as powerful tools in high-throughput proteomics and drug discovery.

The earliest and best-known protein chip is the ProteinChip by Ciphergen Biosystems Inc. (Fremont, Calif.). The ProteinChip is based on the surface-enhanced laser desorption and ionization (SELDI) process. Known proteins are analyzed using functional assays that are on the chip. For example, chip surfaces can contain enzymes, receptor proteins, or antibodies that enable researchers to conduct protein-protein interaction studies, ligand binding studies, or immunoassays. With state-of-the-art ion optic and laser optic technologies, the ProteinChip system detects proteins ranging from small peptides of less than 1000 Da up to proteins of 300 kDa and calculates the mass based on time-of-flight (TOF).

The ProteinChip biomarker system is the first protein biochip-based system that enables biomarker pattern recognition analysis to be done. This system allows researchers to address important clinical questions by investigating the proteome from a range of crude clinical samples (i.e., laser capture microdissected cells, biopsies, tissue, urine, and serum). The system also utilizes biomarker pattern software that automates pattern recognition-based statistical analysis methods to correlate protein expression patterns from clinical samples with disease phenotypes.

In other aspects, the levels of polypeptides in samples can be determined by detecting the biological activities associated with the polypeptides. If a biological function/activity of a polypeptide is known, suitable in vitro bioassays can be designed to evaluate the biological function/activity, thereby determining the amount of the polypeptide in the sample.

### V. Pharmaceutical Compositions

In certain aspects, the compositions or agents for use in the methods, such as therapeutic agents or inhibitors, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body, such as skeletal muscle or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices, local administration, and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain aspects, pharmaceutical compositions may comprise, for example, at least about 0.1% of an active agent, such as an isolated exosome, a related lipid nanovesicle, or an exosome or nanovesicle loaded with therapeutic agents or diagnostic agents. In other aspects, the active agent may comprise between about 2% to about 75% of the weight of the unit, or between about 25% to about 60%, for example, and any range derivable therein. In other non-limiting examples, a dose may also comprise from about 1 microgram/kg/body weight, about microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 microgram/kg/body weight to about 100 mg/kg/body weight, about 5 microgram/kg/body weight to about 500 milligram/kg/body weight, etc., can be administered.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antgifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Topical administration may be particularly advantageous for the treatment of skin cancers, to prevent chemotherapy-induced alopecia or other dermal hyperproliferative disorder. Alternatively, administration may be by orthotopic, intradermal, intralesional, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol is between about 0.01 ml and 0.5 ml.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### VI. Kits

Certain aspects of the present disclosure also concern kits containing compositions of the disclosure or compositions to implement methods of the disclosure. In some aspects, kits can be used to evaluate one or more nucleic acid and/or polypeptide molecules. In certain aspects, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more nucleic acid probes, synthetic RNA molecules or inhibitors, or any value or range and combination derivable therein. In some aspects, there are kits for evaluating gene expression, protein expression, or protein activity in a cell.

In certain aspects, the kits may comprise materials for analyzing cell morphology and/or phenotype, such as histology slides and reagents, histological stains, alcohol, buffers, tissue embedding mediums, paraffin, formaldehyde, and tissue dehydrant.

Kits may comprise components, which may be individually packaged or placed in a container, such as a tube, bottle, vial, syringe, or other suitable container means.

Individual components may also be provided in a kit in concentrated amounts; in some aspects, a component is provided individually in the same concentration as it would be in a solution with other components. Concentrations of components may be provided as 1×, 2×, 5×, 10×, or 20× or more.

Kits for using probes, polypeptide detecting agents, and/or inhibitors or agents of the disclosure for prognostic or diagnostic applications are included. Specifically contemplated are any such molecules corresponding to any nucleic acid or polypeptide identified herein.

In certain aspects, negative and/or positive control agents are included in some kit aspects. The control molecules can be used to verify transfection efficiency and/or control for transfection-induced changes in cells.

Aspects of the disclosure include kits for analysis of a pathological sample by assessing a nucleic acid or polypeptide profile for a sample comprising, in suitable container means, two or more RNA probes, or a polypeptide detecting agent, wherein the RNA probes or polypeptide detecting agent detects nucleic acids or polypeptides described herein. Furthermore, the probes, detecting agents and/or inhibiting reagents may be labeled. Labels are known in the art and also described herein. In some embodiments, the kit can further comprise reagents for labeling probes, nucleic acids, and/or detecting agents. The kit may also include labeling reagents, including at least one of amine-modified nucleotide, poly(A) polymerase, and poly(A) polymerase buffer. Labeling reagents can include an amine-reactive dye. Certain aspects also encompass kits for performing the diagnostic or therapeutic methods. Such kits can be prepared from readily available materials and reagents. For example, such kits can comprise any one or more of the following materials: enzymes, reaction tubes, buffers, detergent, primers, probes, antibodies. In a particular aspect, these kits allow a practitioner to obtain samples of neoplastic cells in breast, blood, tears, semen, saliva, urine, tissue, serum, stool, sputum, cerebrospinal fluid and supernatant from cell lysate. In another particular aspect, these kits include the needed apparatus for performing RNA extraction, RT-PCR, and gel electrophoresis. Instructions for performing the assays can also be included in the kits.

In a particular aspect, these kits may comprise a plurality of agents for assessing the differential expression of a plurality of biomarkers, wherein the kit is housed in a container. The kits may further comprise instructions for using the kit for assessing expression, means for converting the expression data into expression values and/or means for analyzing the expression values to generate prognosis. The agents in the kit for measuring biomarker expression may comprise a plurality of PCR probes and/or primers for qRT-PCR and/or a plurality of antibody or fragments thereof for assessing expression of the biomarkers. In another aspect, the agents in the kit for measuring biomarker expression may comprise an array of polynucleotides complementary to the mRNAs of the biomarkers. Possible means for converting the expression data into expression values and for analyzing the expression values to generate scores that predict survival or prognosis may be also included.

Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific prognostic or non-prognostic application, and may also indicate directions for either in vivo or in vitro use, such as those described above. The kit may comprise the container described above and one or more other containers comprising materials desirable from a commercial and user standpoint, including buffers, diluents, filters, needles, syringes, and package inserts with instructions for use.

### VII. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Melanoma Cells with BRAF Amplification-Mediated Dual BRAFi+MEKi Resistance Show Increased Sensitivity to Ferroptosis

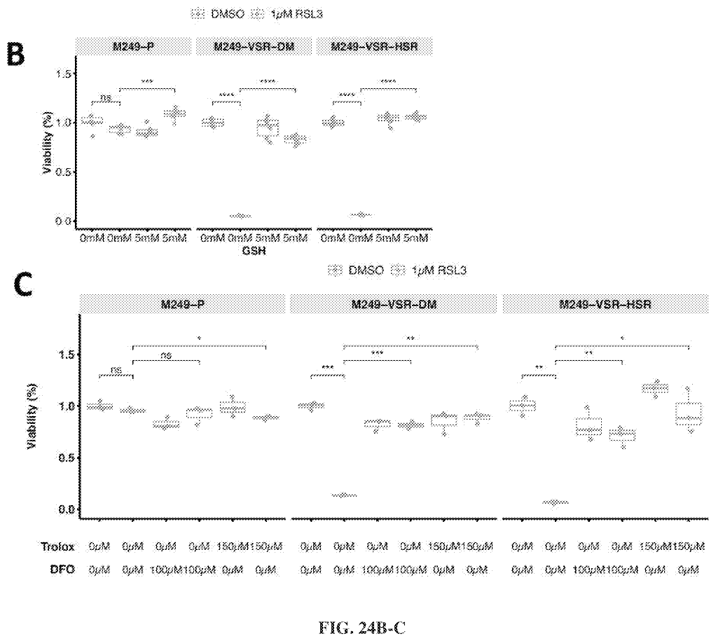

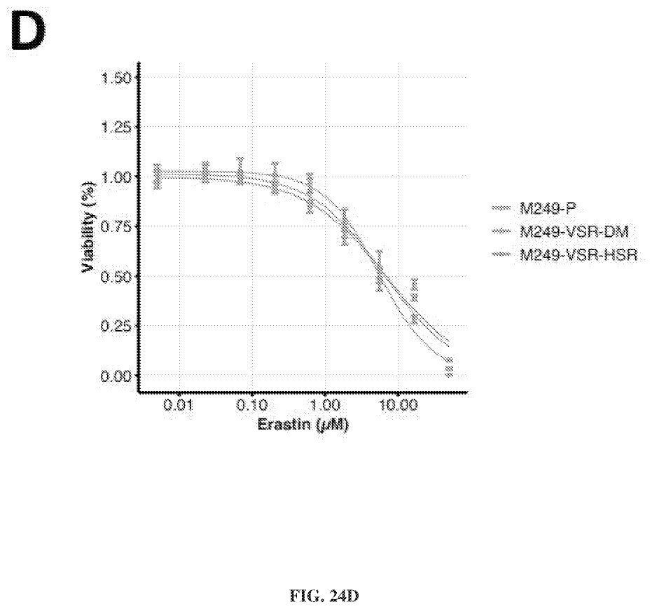

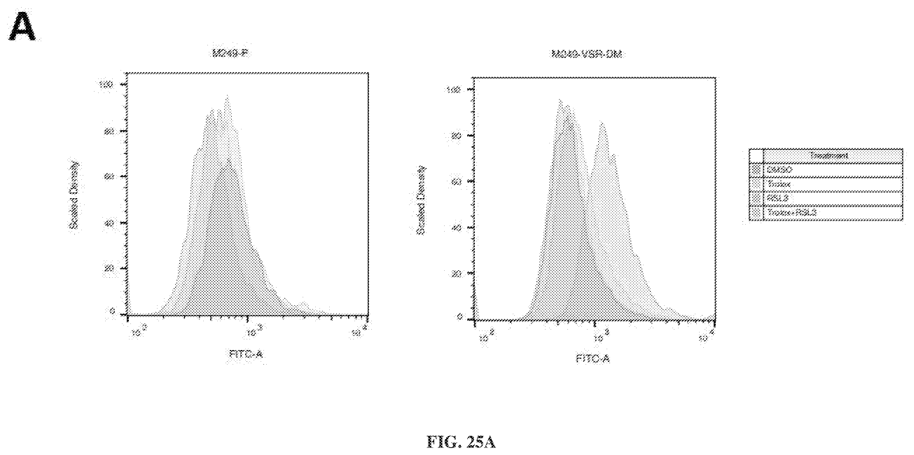

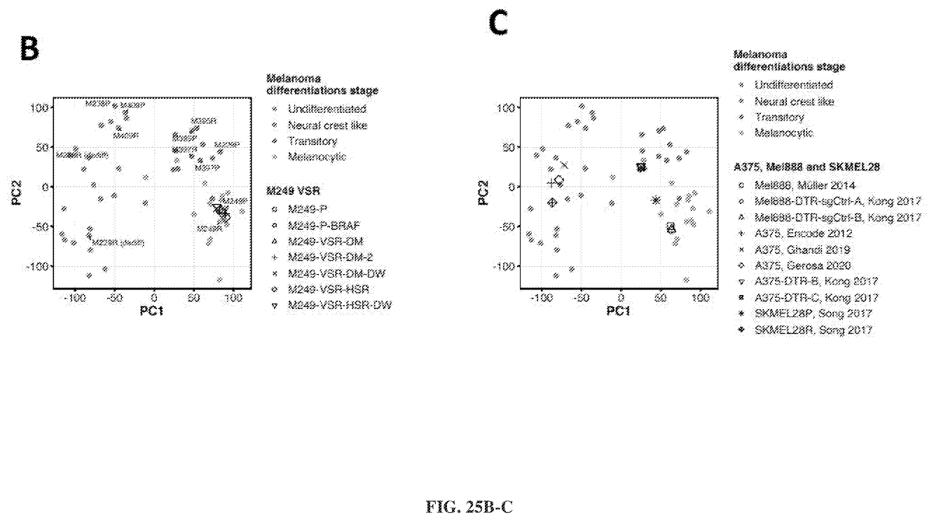

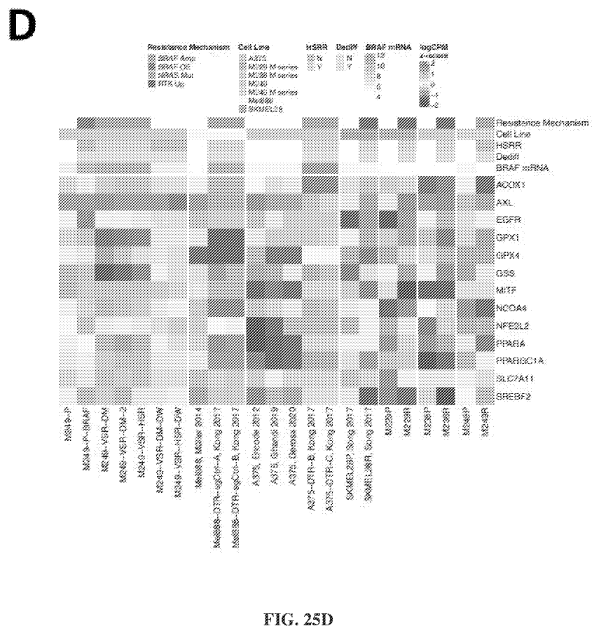

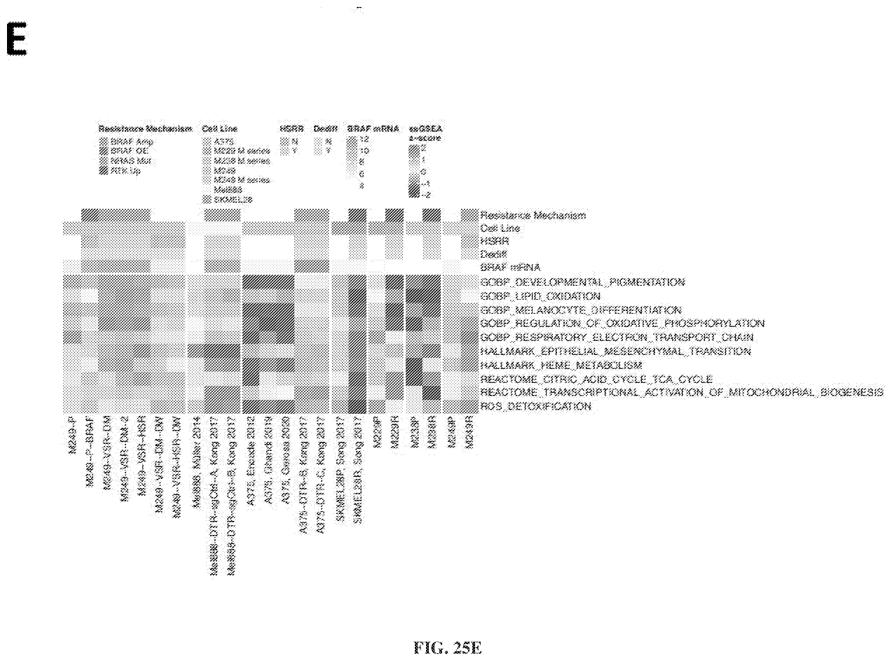

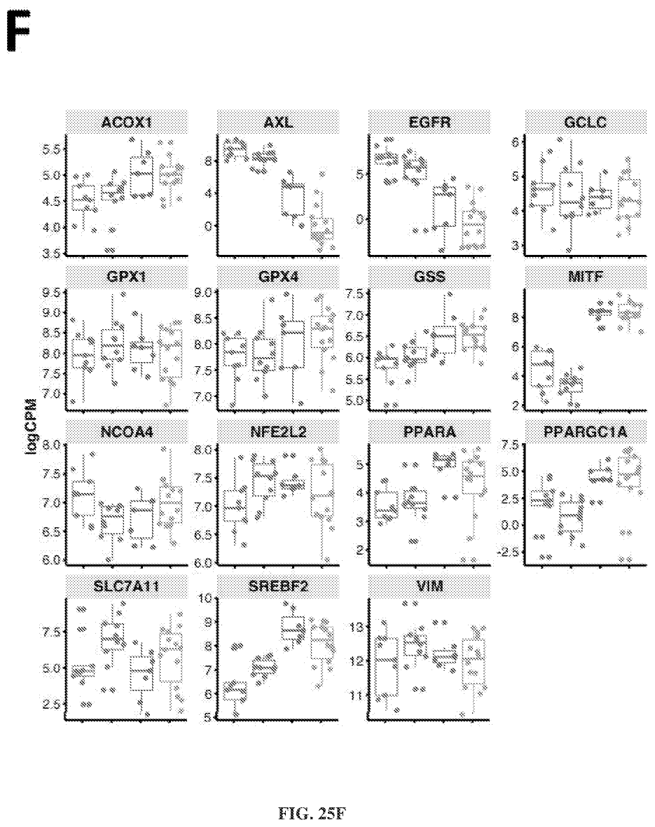

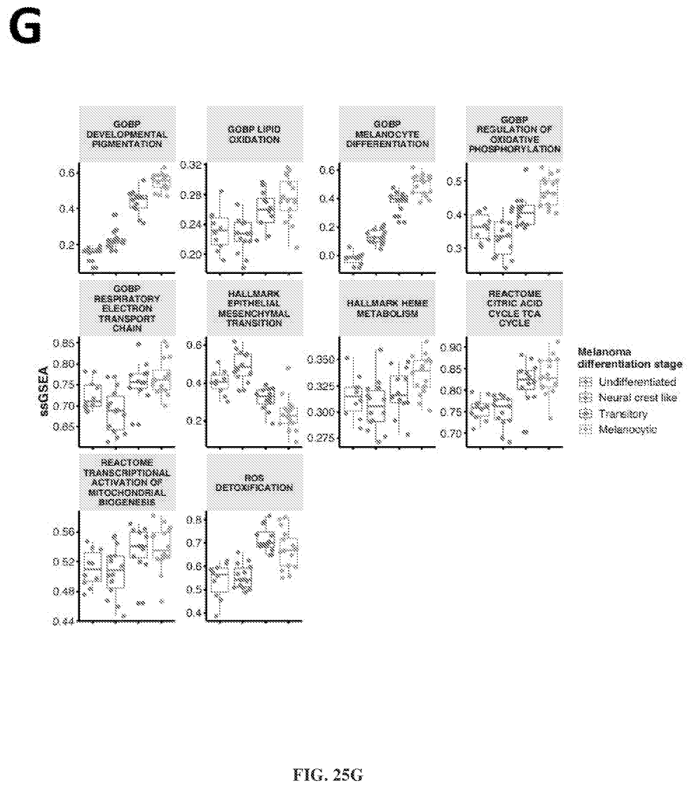

Given the high plasticity of BRAF amplifications in the M249-VSR series, the inventors next investigated cellular vulnerabilities affiliated with amplification in this dual MAPKi context. Previous studies revealed a correlation between melanoma differentiation stages and sensitivity to pro-ferroptotic drugs (1). The inventors thus tested if BRAF amplified cells have altered sensitivity to disruption of the repair of oxidized lipids. The inventors tested the sensitivity of the pro-ferroptotic drug RSL3, which targets glutathione peroxidase 4 (GPX4), in the M249-VSR series. The inventors found that both M249-VSR-DM and —HSR are substantially more sensitive to RSL3 compared to M249-P (FIG. 24A). The inventors further found that after drug withdrawal, when the cells have reduced BRAF copy number, M249 cells lose sensitivity reverting to levels closer to the parental cells. Consistently, two additional cases of BRAFi+MEKi-resistance mediated by BRAF amplification (A375-DTR and Mel888-DTR; both lines dabrafenib (BRAFi)+trametinib (MEKi) resistant (DTR) (2)) also showed increased RSL3 sensitivity compared to their parentals. The inventors next confirmed that the RSL3 sensitivity in M249-VSR sublines have the expected characteristics of ferroptosis. Namely that the RSL3 sensitivity is reactive oxidative stress (ROS)-, lipid ROS-, and iron-dependent, as cell death can be rescued by adding reduced glutathione (GSH), the lipophilic antioxidant Trolox, and the iron chelator deferoxamine (DFO) (FIGS. 24B and 24C). The inventors measured lipid ROS levels in M249-P and M249-VSR cells using C11-BODIPY dye and found increased lipid ROS levels upon RSL3 treatment, that was protected by the presence of Trolox (FIG. 25A).

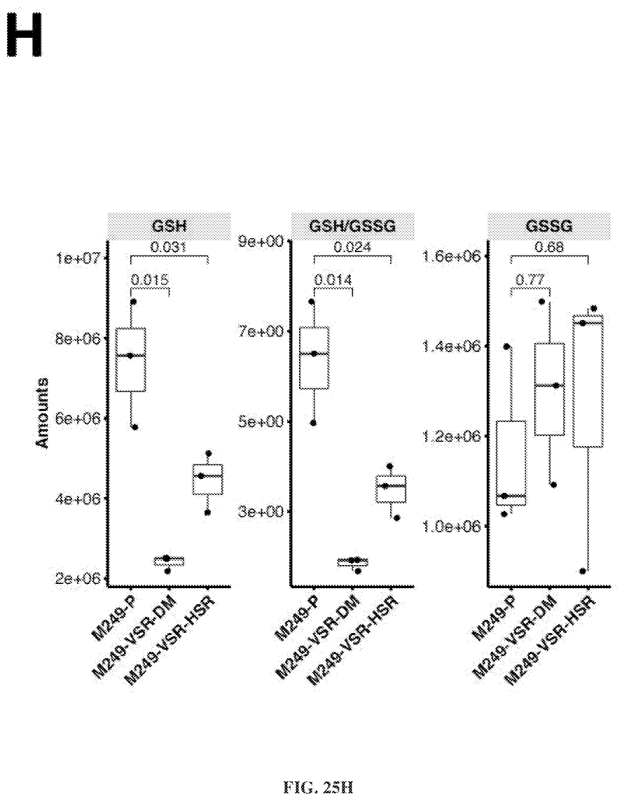

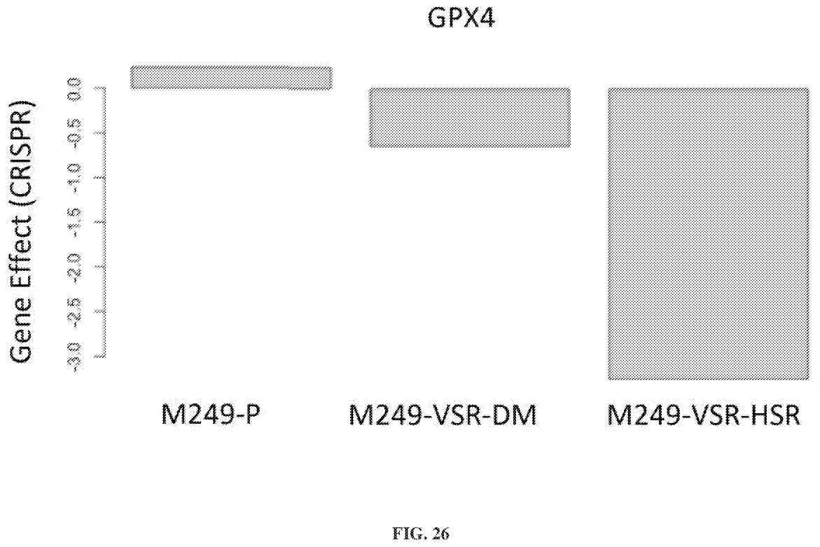

The BRAF-amplified M249 dual MAPKi-resistant cells that are sensitive to the GPX4 inhibitor RSL3 were also sensitive GPX4 knockout (FIG. 26), but were not more sensitive to inhibition of the system x_{c}⁻ cystine/glutamate antiporter by Erastin (FIG. 24D). Such differential sensitivity to different upstream components of the glutathione synthesis and ferroptosis pathway have been previously observed (e.g., SKMEL28R in Tsoi, et al (1)).

The inventors next investigated why these three cell lines with BRAF amplification- and thus MAPK reactivation-mediated resistance demonstrated higher ferroptosis sensitivity compared to their parental sublines. In previous studies, ferroptosis sensitivity in melanoma is associated with innate or acquired treatment-induced dedifferentiation (1). However, in the inventors' past work, resistance mediated by reactivation of the MAPK pathway through genomic changes (e.g., via NRAS mutation: M249P/R), does not lead to dedifferentiation and changes in ferroptosis sensitivity (FIG. 25B). The inventors thus analyzed whether the three BRAF-amplified dual MAPKi-resistant cells studied here demonstrated signs of dedifferentiation. Gene expression profiles of RSL3 sensitive M249-VSR (DM and HSR amplification mode), A375-DTR and Mel888-DTR cells (both with HSR mode) do not demonstrate dedifferentiation compared to their parental sublines when their gene expression profiles are projected onto a panel of melanoma lines spanning the full spectrum of differentiation states (1). By contrast, cell lines M229P/R, M238P/R and SKMEL28P/R, which became resistant through upregulation of RTKs (3), did demonstrate dedifferentiation and increased sensitivity to RSL3 (as tested previously (1)) (FIG. 25B-C). Such findings are also supported by the combination of increases in melanocyte differentiation and pigmentation, decreases in mesenchymal gene set scores, and increases in the melanoma differentiation master regulator MITF in the BRAF amplification samples, and the reverse patterns in the RTK upregulation/dedifferentiation cases (FIG. 25D-G).

Upon determination that increased RSL3 sensitivity in these three BRAF amplification cases are not due to dedifferentiation, the inventors then turned their focus to mitochondrial pathways as previous studies have reported that MAPKi resistance can cause melanoma cells to shift their major energy generation program from glycolysis to mitochondrial pathways. This shift then leads to elevated production of ROS and more dependence on ROS detoxifying mechanisms (4-8). Although ROS were implicated, these prior studies did not assess the change in ferroptosis sensitivity of the resistant sublines. The inventors found that upon acquisition of BRAFi/MEKi resistance M249, Mel888 and A375 all upregulate PPARGC1A (PGC1-α) (4), have distinct but overlapping patterns of upregulation of mitochondrial respiration programs (tricarboxylic acid cycle (TCA), electron transport chain (ETC), oxidative phosphorylation, and mitochondrial biogenesis), and all upregulate lipid oxidation pathways (featured by PPARα and ACOX1(9)). In sum, these changes may cause higher dependence on glutathione metabolism for lipid detoxification via GPX4, while all cases do not equally upregulate their ROS detoxification pathways (FIG. 25D-E).

In accordance with this last observation, i) expression of the ROS detoxification pathway gene glutathione synthetase (GSS) and inferred activity of the ROS detoxification pathway are downregulated in both dedifferentiation and BRAF amplification cases of MAPKi resistance (FIG. 25F-G), and ii) the levels of reduced glutathione (GSH) are decreased upon both dedifferentiation (1) and upon BRAF amplification (FIG. 25H). The inventors furthermore found that the NCOA4 (nuclear receptor coactivator 4) gene, that mediates the selective autophagic degradation of ferritin (10) is upregulated in both dedifferentiation- and BRAF amplification-mediated MAPKi resistant cells, but is downregulated in NRAS mutation-mediated resistance (where both parental and resistant sublines have similar ferroptosis sensitivity (1)). This observation is in line with a previous study finding that NCOA4 promotes accumulation of cellular labile iron, leading to higher susceptibility to pro-ferroptotic drugs

Taken together, although dedifferentiation-mediated MAPKi resistance has distinctions from BRAF amplification-mediated resistance, they both demonstrate increased GPX4 inhibition (RSL3) sensitivity, have a common pattern of downregulated ROS detoxification genes such as GSS, and demonstrate upregulation of the iron homeostasis regulator NCOA4. Furthermore, the absence of such patterns is observed in MAPKi resistance cases that do not demonstrate increased RSL3 sensitivity.

The finding of increased ferroptosis sensitivity in BRAF-amplified melanomas relapsed from dual MAPKi treatment extends the spectrum of ferroptosis sensitivity in melanoma therapy. The inventors found that additional mechanisms, distinct from treatment-induced dedifferentiation and mesenchymal transition, can also generate sensitivity to GPX4 inhibition (1). This finding links to studies of MAPKi-induced oxidative stress in melanoma. In some melanomas, BRAF V600E activation leads to enhanced glycolysis and reduced oxidative phosphorylation and mitochondrial respiration 4). However, BRAF inhibition, including acquired resistance to BRAF inhibitors, can switch the energy generation dependency back to oxidative phosphorylation pathway by induction of PPARGC1A and overexpression of other mitochondrial genes (4-7, 11, 12). Reactive oxygen species (ROS) productively mediate redox-based energy production in mitochondrial respiration, but they can also damage lipid, protein and DNA. (13) Hence respiring cells need to upregulate detoxification programs to compensate for elevated oxidative stress (8, 14). The imbalance of cellular prooxidative and antioxidative mechanism can lead to cell death (15). Ferroptosis is one form of cell death that can result from such compromised redox homeostasis, mediated by iron-dependent accumulation of lipid peroxides (16). More specifically, oxidants such as hydrogen peroxide generated through mitochondria respiration can be converted to hydroxyl radicals through the Fenton reaction in presence of ferrous iron. Then hydroxyl radical can then oxidize membrane phospholipids (17). Cells need to leverage glutathione synthesis to combat lipid oxidation (18), through the action of genes such as and glutathione synthetase (GSS) and glutathione peroxidase 4 (GPX4).

In these studies, BRAF amplification-mediated MAPKi resistant melanoma cells did not exhibit dedifferentiation. However, they did downregulate GSS and had limited reduced glutathione levels, which would limit their capacity to detoxify lipid ROS (FIG. 25D). They also upregulated the iron homeostasis regulator NCOA4 (19, 20), similar to other MAPKi parental/resistant melanoma pairs with differential RSL3 sensitivity, consistent with higher vulnerability to ferroptosis induction (FIG. 25D). These finding uncover a different form of cell death, ferroptosis, occurring under a MAPKi resistance context, and extends the role of ferroptosis in MAPKi resistance beyond cases of dedifferentiation. Taken together, the melanoma dedifferentiation-independent synthetic lethality between BRAF amplification and ferroptosis identified here provides therapeutic insight for treating BRAF amplified melanomas relapsed from MAPKi treatment.

### REFERENCES

The following references and the publications referred to throughout the specification, to the extent that they provide exemplary procedural or other details supplementary to those set forth herein, are specifically incorporated herein by reference.
1. Tsoi, J. et al. Multi-stage Differentiation Defines Melanoma Subtypes with Differential Vulnerability to Drug-Induced Iron-Dependent Oxidative Stress. Cancer Cell (2018) doi:10.1016/j.ccell.2018.03.017.
2. Kong, X. et al. Cancer drug addiction is relayed by an ERK2-dependent phenotype switch. Nature 550, 270-274 (2017).
3. Nazarian, R. et al. Melanomas acquire resistance to B-RAF(V600E) inhibition by RTK or N-RAS upregulation. Nature 468, 973-977 (2010).
4. Haq, R. et al. Oncogenic BRAF regulates oxidative metabolism via PGC1α and MITF. Cancer Cell (2013) doi:10.1016/j.ccr.2013.02.003.
5. Cesi, G., Walbrecq, G., Zimmer, A., Kreis, S. & Haan, C. ROS production induced by BRAF inhibitor treatment rewires metabolic processes affecting cell growth of melanoma cells. Mol. Cancer (2017) doi:10.1186/s12943-017-0667-y.
6. Trotta, A. P. et al. Disruption of mitochondrial electron transport chain function potentiates the pro-apoptotic effects of MAPK inhibition. J. Biol. Chem. (2017) doi:10.1074/jbc.M117.786442.
7. Schackel, L. et al. Targeting mitochondrial complex I using BAY 87-2243 reduces melanoma tumor growth. Cancer Metab. (2015) doi:10.1186/s40170-015-0138-0.
8. Vazquez, F. et al. PGC1α Expression Defines a Subset of Human Melanoma Tumors with Increased Mitochondrial Capacity and Resistance to Oxidative Stress. Cancer Cell (2013) doi:10.1016/j.ccr.2012.11.020.
9. Shen, S. et al. Melanoma Persister Cells Are Tolerant to BRAF/MEK Inhibitors via ACOX1-Mediated Fatty Acid Oxidation. Cell Rep. (2020) doi:10.1016/j.celrep.2020.108421.
10. Del Rey, M. Q. & Mancias, J. D. NCOA4-mediated ferritinophagy: A potential link to neurodegeneration. Frontiers in Neuroscience (2019) doi:10.3389/fnins.2019.00238.
11. Corazao-Rozas, P. et al. Mitochondrial oxidative phosphorylation controls cancer cell's life and death decisions upon exposure to MAPK inhibitors. Oncotarget (2016) doi:10.18632/oncotarget.7790.
12. Gopal, Y. N. V. et al. Inhibition of mTORC1/2 overcomes resistance to MAPK pathway inhibitors mediated by PGC1α and oxidative phosphorylation in melanoma. Cancer Res. (2014) doi:10.1158/0008-5472.CAN-14-1392.
13. Schieber, M. & Chandel, N. S. ROS function in redox signaling and oxidative stress. Current Biology (2014) doi:10.1016/j.cub.2014.03.034.
14. Yuan, L. et al. Braf mutant melanoma adjusts to BRAF/MEK inhibitors via dependence on increased antioxidant SOD2 and increased reactive oxygen species levels. Cancers (Basel). (2020) doi:10.3390/cancers12061661.
15. Rahal, A. et al. Oxidative stress, prooxidants, and antioxidants: The interplay. BioMed Research International (2014) doi:10.1155/2014/761264.
16. Dixon, S. J. et al. Ferroptosis: An iron-dependent form of nonapoptotic cell death. Cell (2012) doi:10.1016/j.cell.2012.03.042.
17. Hauck, A. K. & Bernlohr, D. A. Oxidative stress and lipotoxicity. J. Lipid Res. (2016) doi:10.1194/jlr.r066597.
18. Yang, W. S. et al. Regulation of ferroptotic cancer cell death by GPX4. Cell (2014) doi:10.1016/j.cell.2013.12.010.
19. Gao, M. et al. Ferroptosis is an autophagic cell death process. Cell Res. (2016) doi:10.1038/cr.2016.95.
20. Hou, W. et al. Autophagy promotes ferroptosis by degradation of ferritin. Autophagy (2016) doi:10.1080/15548627.2016.1187366.
21. Wang, L. et al. An Acquired Vulnerability of Drug-Resistant Melanoma with Therapeutic Potential. Cell (2018) doi:10.1016/j.cell.2018.04.012.
22. Müller, J. et al. Low MITF/AXL ratio predicts early resistance to multiple targeted drugs in melanoma. Nat. Commun. (2014) doi:10.1038/ncomms6712.
23. Ghandi, M. et al. Next-generation characterization of the Cancer Cell Line Encyclopedia. Nature (2019) doi:10.1038/s41586-019-1186-3.
24. Consortium, E. P. et al. An integrated encyclopedia of DNA elements in the human genome. Nature (2013).
25. Gerosa, L. et al. Receptor-Driven ERK Pulses Reconfigure MAPK Signaling and Enable Persistence of Drug-Adapted BRAF-Mutant Melanoma Cells. Cell Syst. (2020) doi:10.1016/j.cels.2020.10.002.
26. Song, C. et al. Recurrent tumor cell-intrinsic and -extrinsic alterations during mapki-induced melanoma regression and early adaptation. Cancer Discov. (2017) doi:10.1158/2159-8290.CD-17-0401.
27. Barbie, D. A. et al. Systematic RNA interference reveals that oncogenic KRAS-driven cancers require TBK1. Nature (2009) doi:10.1038/nature08460.
28. Tsoi, J, Robert, L, Paraiso, K, Galvan, C, . . . Graeber, TG. (2018). Multi-stage differentiation defines melanoma subtypes with differential vulnerability to drug-induced iron-dependent oxidative stress. Cancer Cell, 33(5), 890-904.e5.
29. Barbie, D. A., Tamayo, P., Boehm, J. S., Kim, S. Y., . . . Hahn, W. C. (2009). Systematic RNA interference reveals that oncogenic KRAS-driven cancers require TBK1 (ssGSEA single sample GSEA gene set enrichment analysis). Nature, 462(7269), 108-112.
30. WO/2019/006005

### Example 2: For at Least these Reasons, the Current Claims are Non-Obvious, and Withdrawal of this Rejection is Requested

Focal amplifications (FAs) can mediate targeted therapy resistance in cancer. Understanding the structure and dynamics of FAs is critical for designing treatments that overcome plasticity-mediated resistance. The inventors developed a melanoma model of dual MAPK inhibitor resistance that bears BRAF^{V600} amplifications through either extrachromosomal DNA/double-minutes (ecDNA/DMs) or intrachromosomal homogenously staining regions (HSRs). Cells harboring BRAF^{V600}E FAs displayed mode switching between DMs and HSRs, from both de novo genetic changes and selection of pre-existing subpopulations. Plasticity is not exclusive to ecDNAs, as cells harboring HSRs exhibit drug addiction-driven structural loss of BRAF amplicons upon dose reduction. FA mechanisms can couple with kinase domain duplications and alternative splicing to enhance resistance. Drug-responsive amplicon plasticity is observed in the clinic, and can involve other MAPK pathway genes, such as RAF1 and NRAS. BRAF FA-mediated dual-MAPKi-resistant cells are more sensitive to pro-ferroptotic drugs, extending the spectrum of ferroptosis sensitivity in MAPKi-resistance beyond cases of dedifferentiation.

Understanding the structure and dynamics of oncogene amplifications is critical for overcoming tumor relapse. BRAF amplifications are highly plastic under MAPKi dosage challenges in melanoma, through involvement of de novo genomic alterations, even in the HSR mode. Moreover, BRAF FA-driven, dual-MAPKi-resistant cells extend the spectrum of resistance-linked ferroptosis sensitivity.

### A. Introduction

Genomic instability confers cancer cells with a growing list of hallmarks such as enhanced invasion and deregulated cellular energetics (1). Among many types of instability-driven mutations, focal amplifications (FAs) of oncogenes in cancer genomes is a major contributor of neoplastic progression and therapeutic resistance (2-5). There are primarily two modes of FA structural topology: double minute (DM) and homogeneously staining region (HSR). DMs are circular extrachromosomal DNAs (ecDNAs) which allow copies of oncogenes to exist freely in nuclei and retain intact, altered or even elevated transcription activity due to high chromatin accessibility, enhancer hijacking and formation of transcription hubs (6-12). DMs are able to replicate autonomously, but are acentric and therefore segregate into daughter cells randomly (13-16). HSRs are intrachromosomal amplifications resulting in long segments with uniform staining intensities in cytogenetics (17). Several models regarding the generation and interchange of these two kinds of FAs have been proposed, including but not restricted to episomal, chromothripsis, breakage-fusion-bridge and integration mechanisms (18,19,28,29,20-27). The high prevalence of both kinds of FAs support their importance in tumorigenesis (15). DMs have been observed in large number of tumors of different types, especially in glioblastomas (˜55% by WGS inferred ecDNA (30)) and neuroblastomas (˜31.0% by cytogenetics (31)), but rarely in normal tissues. A high occurrence of the HSR form of FAs is found in particular cancer types such as squamous cell carcinoma and oral cavity (12.1% and 10.9% by cytogenetics), but across all cancers HSRs have a slightly lower frequency compared to DMs (31).

Mutations in BRAF, a serine/threonine RAF family kinase and a key upstream member of the MAPK pathway, have been associated with many cancer types. The frequency of BRAF mutations varies widely across cancer types. For example, BRAF mutations are relatively common in thyroid cancer and skin melanoma (60% and 52% respectively), but are very rare in kidney cancers (0.3%), based on the TCGA database. In melanoma therapy, the development of inhibitors targeting the BRAF^{V600}E mutation, such as vemurafenib and dabrafenib as well as combinatorial treatments with other MAPK pathway inhibitors (MAPKi) have greatly improved patient survival (32). However, acquired resistance often compromises the efficacy of these therapies. To date, many resistance mechanisms to BRAF inhibition emerging during clinical treatment have been identified, including reactivation of the MAPK pathway, activation of the PI3K/AKT pathway, or both. This can occur via genomic mutations, genomic rearrangements such as kinase domain duplication, altered splice isoform variant expression, and cellular dedifferentiation (5,33-41). One mechanism of reactivating the MAPK pathway that is frequently found in melanoma patient tumors is the acquisition of BRAF amplifications (5). As previously noted, these amplifications can be mediated through both DM and HSR FA modes (35,42,43). However, the details related to the generation, structure, dynamics, plasticities and vulnerabilities of MAPK FAs due to acquired drug resistance in melanoma are incomplete, and as such are the focus of the current study.

In this study, through acquired BRAF and MEK inhibitor resistance, the inventors developed a melanoma model system that dynamically harbors mutant BRAF^{V600}E in the form of DMs, HSRs or both. Using single-cell-derived clones, the inventors found that increasing and/or decreasing kinase inhibitor dosage is a reproducible modulator of the number of DMs, the length of HSRs, the transition between these FA modes, and coupling with additional genomic rearrangements such as kinase domain duplications and alternative splicing. Moreover, the inventors observed plasticity of FAs involving other amplified MAPK genes such as RAF1 and NRAS in NRAS^{MUT} melanoma. Using optical mapping (OM) and whole genome sequencing (WGS), the inventors profiled the BRAF FA structures and found conserved amplicon boundaries between the DM and HSR modes. Furthermore, the observed junction sequences yielded initial insight into the mechanisms of integration and HSR shortening. In investigating the cellular liabilities of BRAF amplification, the inventors identified an increased sensitivity to ferroptosis via GPX4 inhibition, which extends the spectrum of melanoma resistance-derived ferroptosis sensitivity beyond cases of dedifferentiation. Collectively, these findings on BRAF amplicon structure, DM and HSR plasticity, and potential vulnerabilities associated with BRAF FA-driven resistance, highlight key therapeutic challenges and opportunities.

### B. Results

### 1. Acquired Resistance to BRAF and MEK Kinase Inhibitors Resulted in Both DM and HSR Karyotypes

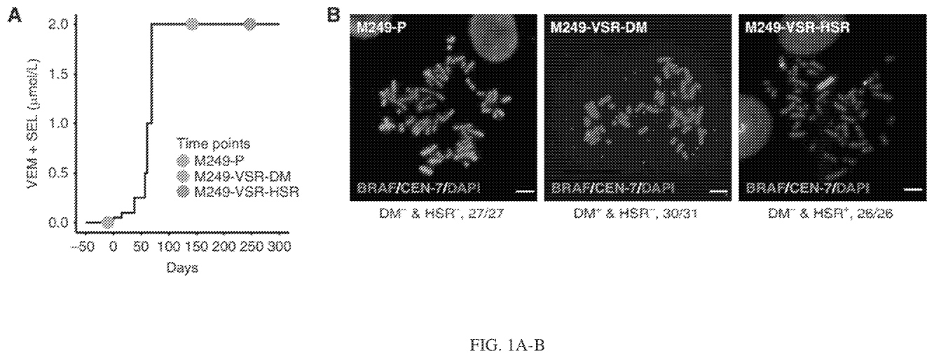

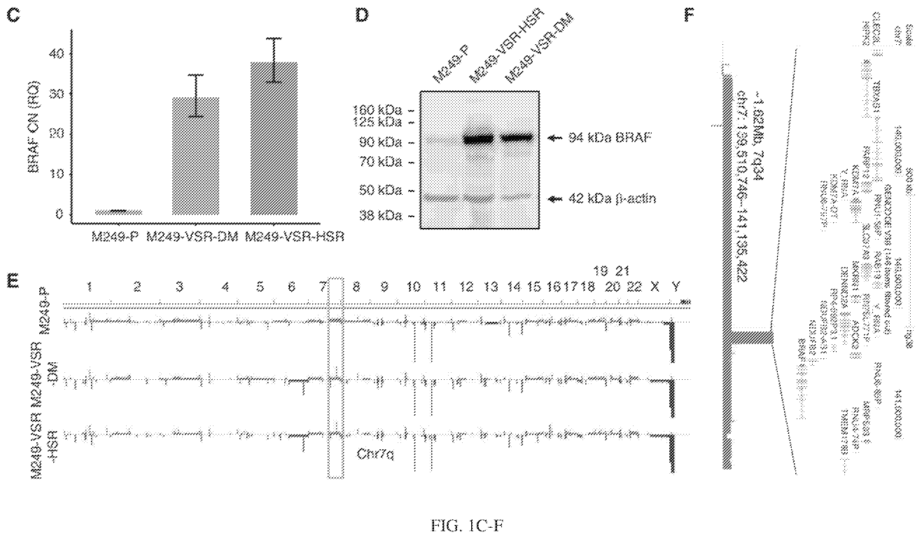

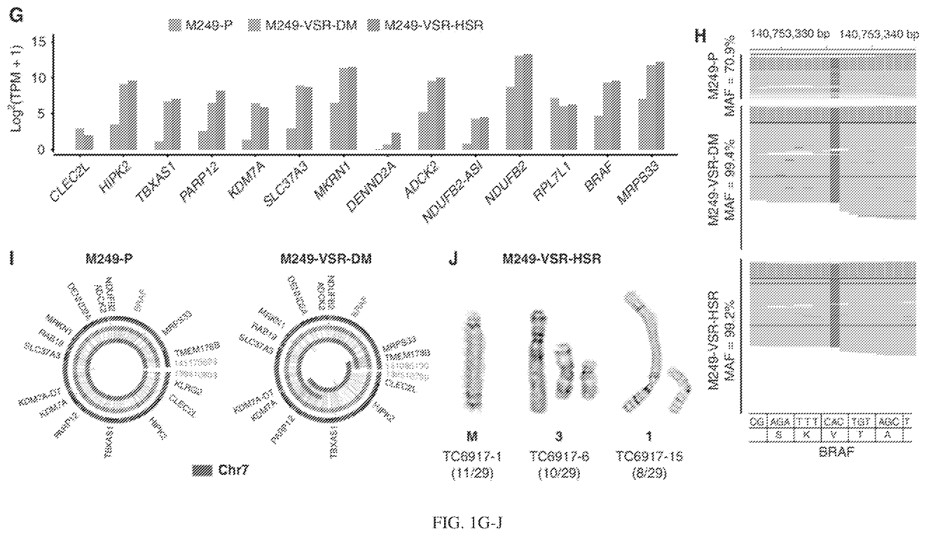

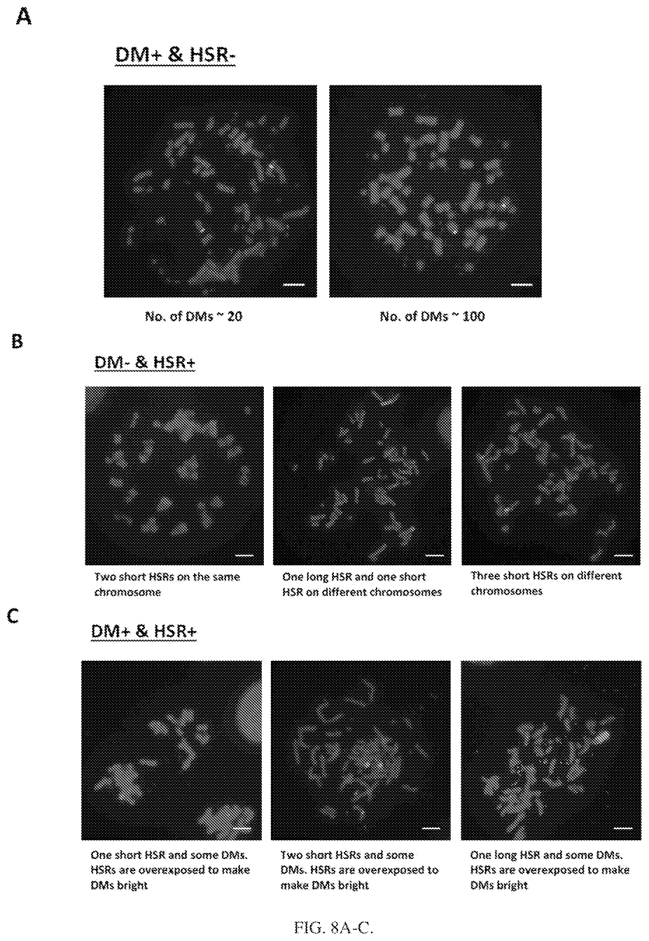

In order to generate a FA-positive melanoma model, the inventors treated a BRAF^{V600}E human melanoma cell line, M249 with vemurafenib (BRAF inhibitor, BRAFi) and selumetinib (MEK inhibitor, MEKi) to develop resistance (abbreviated as M249-VSR for vemurafenib and selumetinib resistant) as previously described (41) (FIG. 1A). Upon the establishment of cells resistant at 2 μM, fluorescent in situ hybridization (FISH) was performed and showed a high amplification of BRAF, primarily in the DM/ecDNA form. However, over the course of a few months in culture, these cells spontaneously switched their karyotypes to DM-negative and HSR-positive with a small number of exceptions (FIG. 1B. See FIG. 8A-C and Methods section for categories and images of BRAF FA FISH-based karyotypes). To quantify the extent of FA, the inventors performed quantitative real time PCR (qPCR) on M249-VSR-DM and -HSR cells and found that there were 30- to 40-fold increases in BRAF copy number compared to M249 parental cells which contained 5 copies of BRAF (FIG. 1B-C). These amplifications also led to high protein levels of BRAF (FIG. 1D).

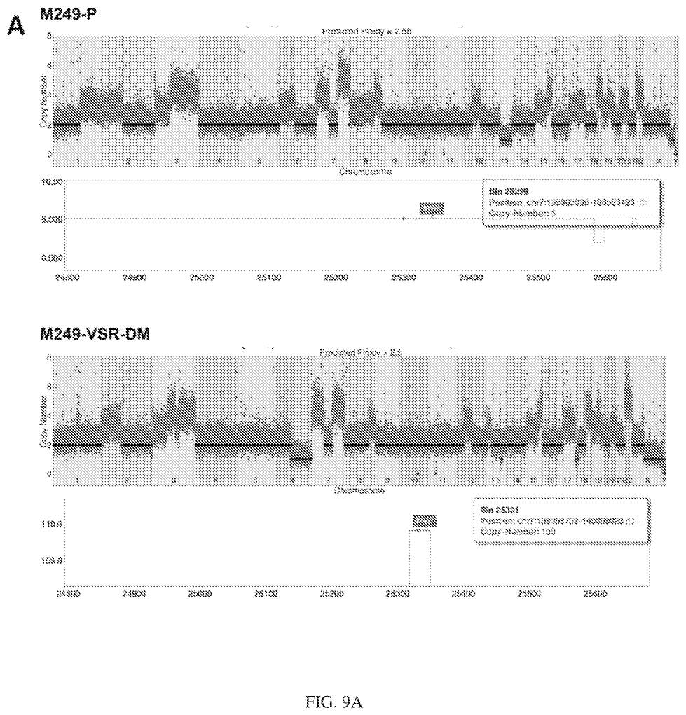

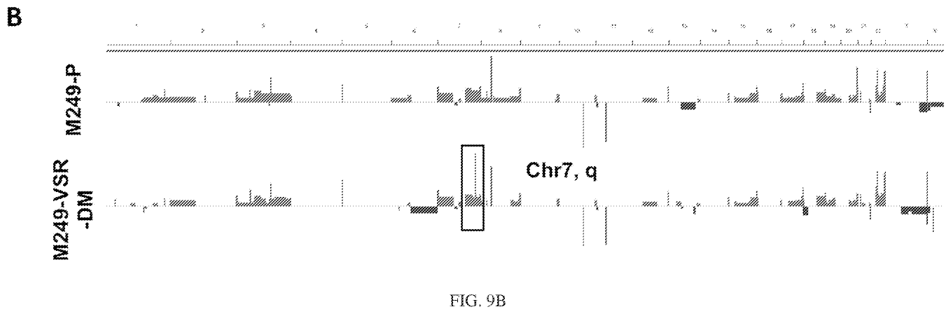

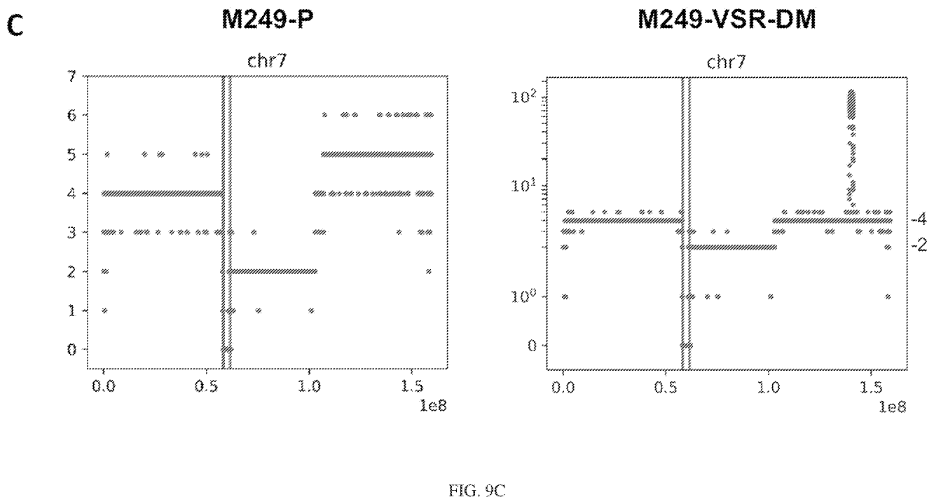

Since qPCR is limited to investigating a small DNA region, the inventors employed WGS and comparative genomic hybridization (CGH) for M249-P and M249-VSR cells to reveal the full copy number alterations/variations (CNA) across the genome (FIG. 1E and FIG. 9A-C). Though there were other alterations, the most striking change upon acquisition of resistance was a FA of size ˜1.62 Mb at chr7q34, the region of the BRAF locus, with a fold increase consistent with qPCR results. The amplicon had highly similar start and end points in both the DM and HSR modes of amplification, supporting that the ensuing HSRs were generated through integration of DMs. Genes adjacent to BRAF on the amplicon were amplified to a similar degree (FIG. 1F); and the transcripts of these genes were also elevated as measured by RNA-seq (FIG. 1G). Such co-amplifications have also been found on amplicons containing other oncogenes, e.g. MYC and EGFR (11,12,44). RNA-seq based single nucleotide variants (SNV) calling of DM and HSR M249 cell lines indicated that the BRAF 1799T>A (V600E) mutation was selected during FA development, with both DM and HSR cases displaying greater than 99% major allele frequency compared to 71% in the parental line (FIG. 1H).

The inventors next characterized the structure of DM and HSR amplicons, aided by the inclusion of optical mapping (OM) data. The observed OM junctions confirmed the circular structure (6,30,45) of the DMs/ecDNAs generated during the acquisition of resistance. In contrast, the parental M249 cells with 5 BRAF copies per cell show a linear arm level amplification (FIG. 1I and FIG. 9C). For HSRs, the inventors investigated the sites of integration. Through cytogenetic G-banding the inventors found a limited level of heterogeneity of HSR integration sites, with integration on either chromosome 1 or 3, or on one or more marker (unidentifiable by G-banding) chromosomes (FIG. 1J).

2. Single-Cell-Derived Clones Confirm De Novo Integrations of DMs into Chromosomes as HSRs

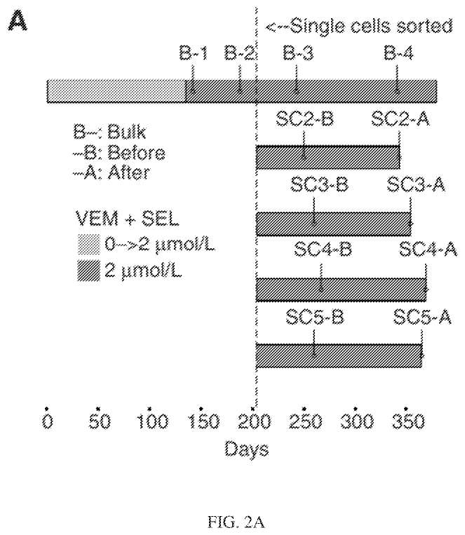

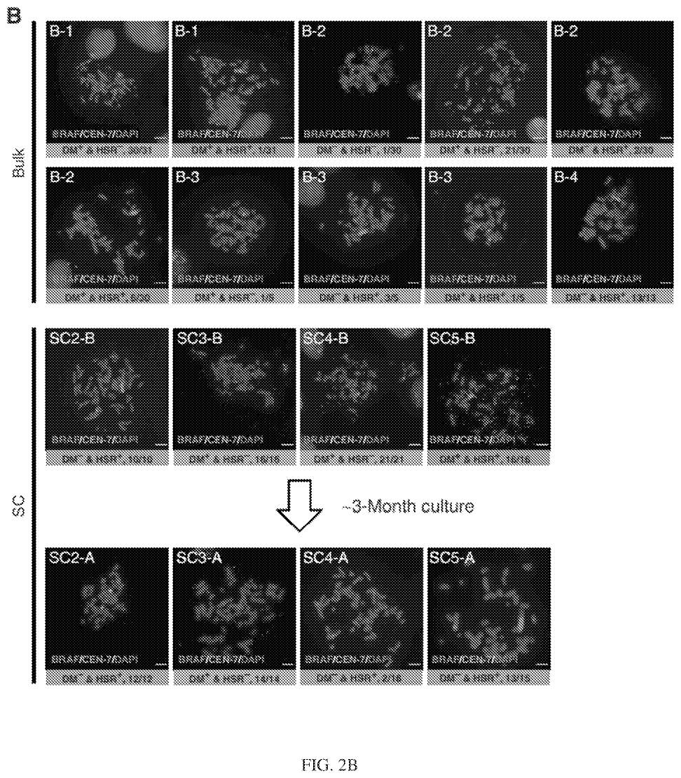

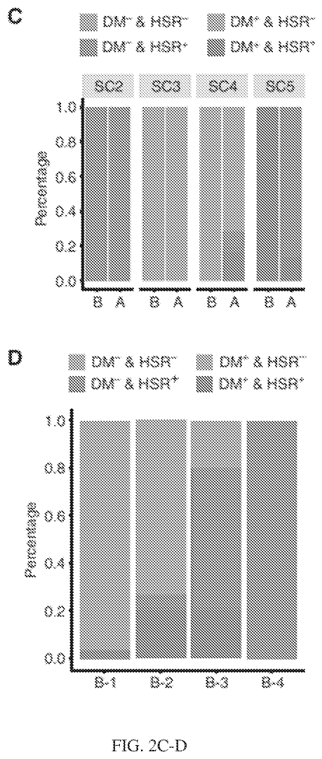

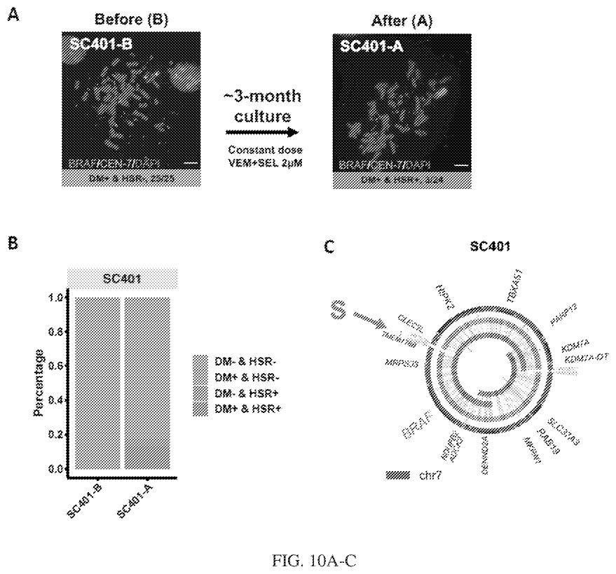

To further dissect changes that occurred during the transition from DMs to HSRs, the inventors isolated single-cell-derived clones (SCs) from the bulk M249-VSR population at an intermediary timepoint between the DM+ & HSR− and DM− & HSR+ karyotypes (FIG. 2A). Cultures derived from these clones were expanded and characterized for subsequent changes over a three-month timeframe. At the outset, three of the resultant clones had a DM+ & HSR-karyotype (SC3, SC4, and SC401), one clone had a DM− & HSR+ karyotype (SC2), and one had a DM+ & HSR+karyotype (SC5) (FIG. 2B, C and FIG. 10A-D). Over the matching three-month time course, the bulk population began with a small percentage of DM− & HSR+ cells which gradually expanded to dominate the population (FIG. 2B, D, B1-B4). The SCs experiments de-convoluted such changes by displaying a range of evolutionary trajectories that implicated de novo DM integration as HSRs, followed by selection of HSR+ cells.

First, although the majority of SC4 and SC401 cells kept their DM+ & HSR-karyotype, some cells began to have the DM− & HSR+ karyotype and some cells harbored both DMs and HSRs (FIGS. 2B, C and 10A-B). Second, HSR+ cases of some SC4 cells presented in a format that had three smaller HSR segments in different chromosomes in each cell (FIGS. 2B and C), while only one long HSR, with or without an additional short HSR segment, was observed in M249-VSR-HSR bulk cells. These single cell clone dynamics support de novo integration of DMs as HSRs. A previous study reported that non-homologous end joining (NHEJ) is needed for efficient DM formation (22). The inventors found that NHEJ may also participate in ecDNA integration (see also the integration junction analysis results). Long-term treatment of SC4 cells with the DNA-PK (also known as PRKDC, a key NHEJ kinase) inhibitor NU7026 at a dose that minimally affects growth (22) decreased the frequency of cells displaying HSR integrations (FIG. 2E). Third, a more pronounced FA mode switch was observed in SC5: with 87% of the cells switching from DM+ & HSR+ to DM− & HSR+, and only 13% retaining the mixed karyotype (FIGS. 2B and C). Cells with DM+ & HSR+ karyotypes appear to reflect an intermediate transition stage in the karyotype switch. In contrast to the initial DM+ cases, clone SC2 that only contained HSRs on chromosome 3 at the outset (FIG. 2F) maintained the HSR mode for three months in culture (FIGS. 2B and C). However, the inventors did detect HSR plasticity in some cells in terms of duplications and/or translocations of shorter versions of the HSR to other chromosomes, with the concurrent retention of the long HSR (FIG. 2B, 9A). Furthermore, long-term inhibition of DNA-PK in the HSR subclone SC2 lead to a lower percentage of multiple HSRs, implicating a role for NHEJ in HSR plasticity (FIG. 2G).

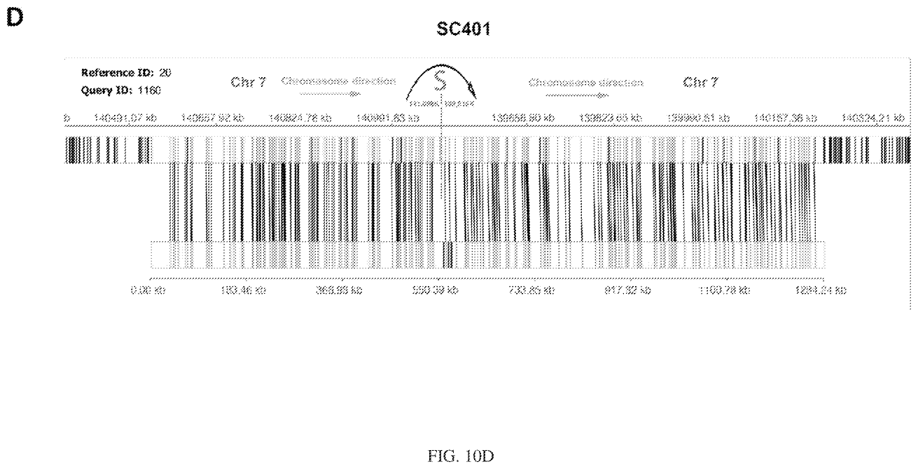

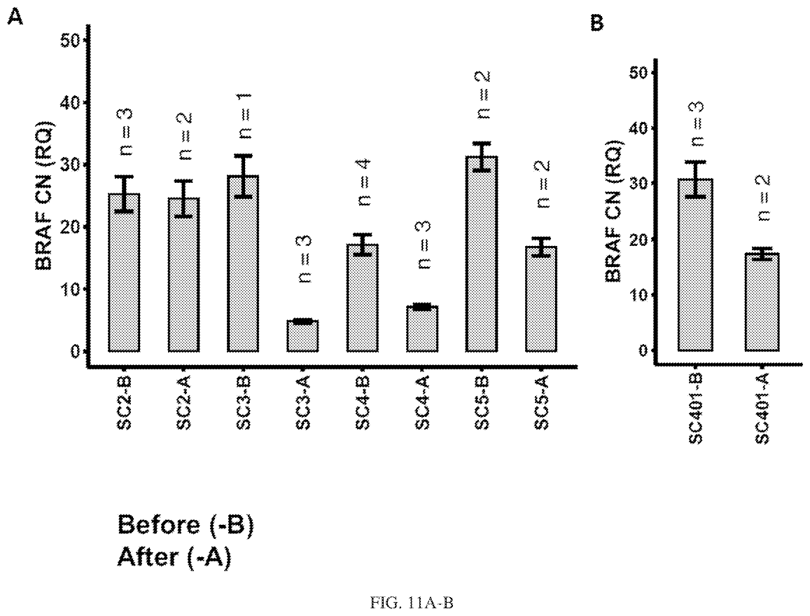

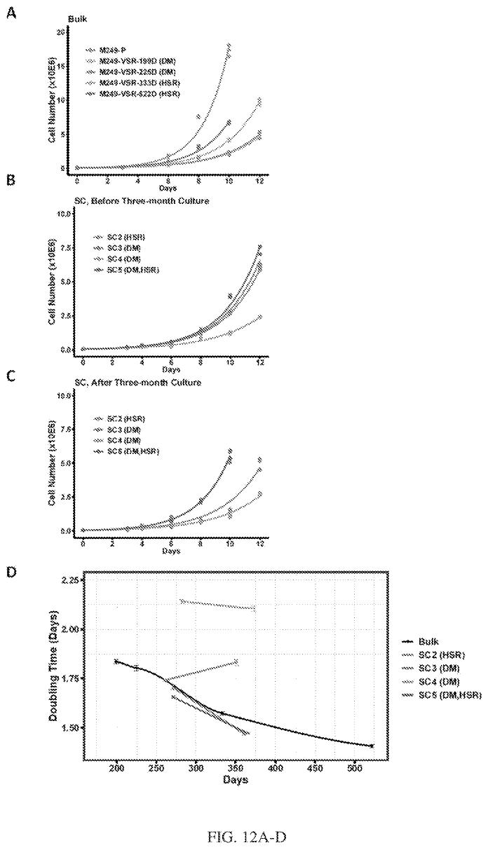

A distinction between the DM and HSR modes of FA was observed during SC long-term culture, in that all subclones with DMs had their BRAF copy number decrease while the BRAF copy number in HSR subclones remained unchanged (FIG. 11A-B). In addition to de novo karyotype changes, the inventors observed heterogeneity and changes in growth rates over three-month expansion among the SCs (FIG. 12A-D).

The data above support that de novo integration of BRAF DMs as HSRs did occur under the steady dose of dual MAPKi treatment, likely due to HSRs being a more stable and/or fit mode of FA in the melanoma MAPKi BRAF amplification context. These results are in line with prior findings in different cell types and different treatments (21,25,28,29). However, no changes occurred in one DM+ & HSR− clone, SC3, indicating that the tendency for integration is not absolute during the time scale observed (FIGS. 2B and C). In sum, these SCs-based findings demonstrate the plasticity of MAPKi-induced BRAF FAs, with a general trend of fitness-based evolution from DMs to HSRs in these conditions.

3. Non-Steady Dose Challenge can Prolong or Prevent DM Integration into Chromosomes

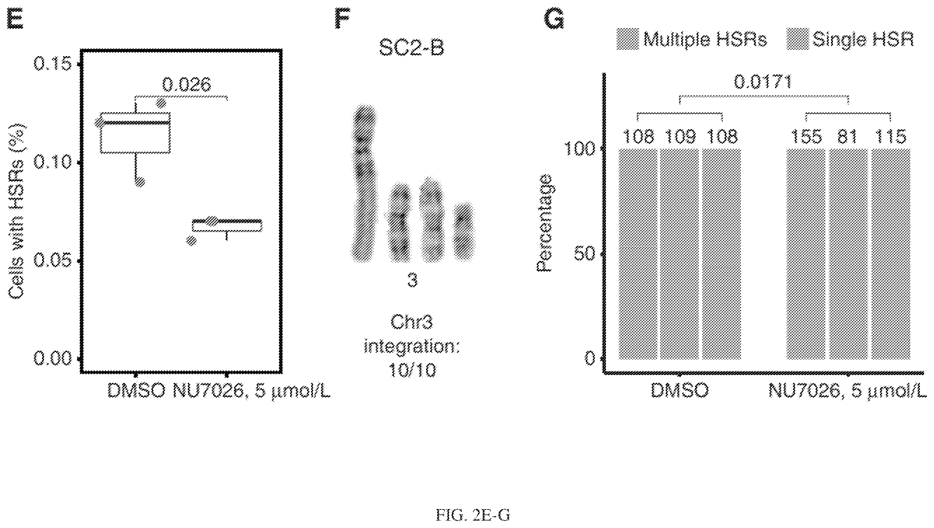

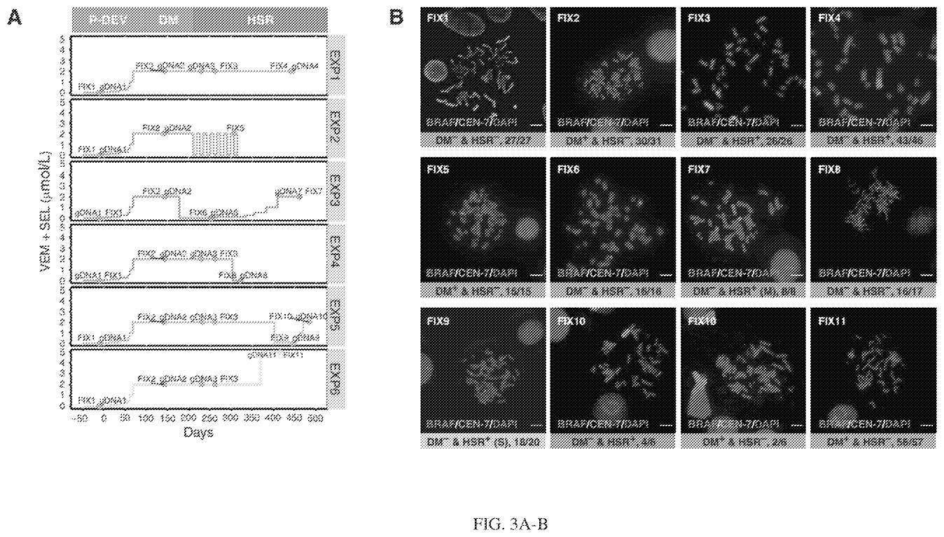

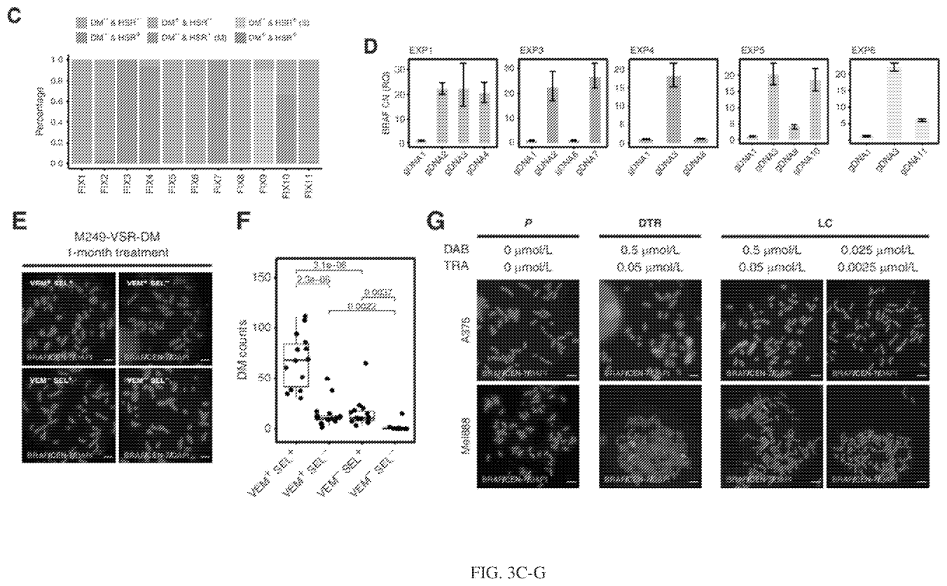

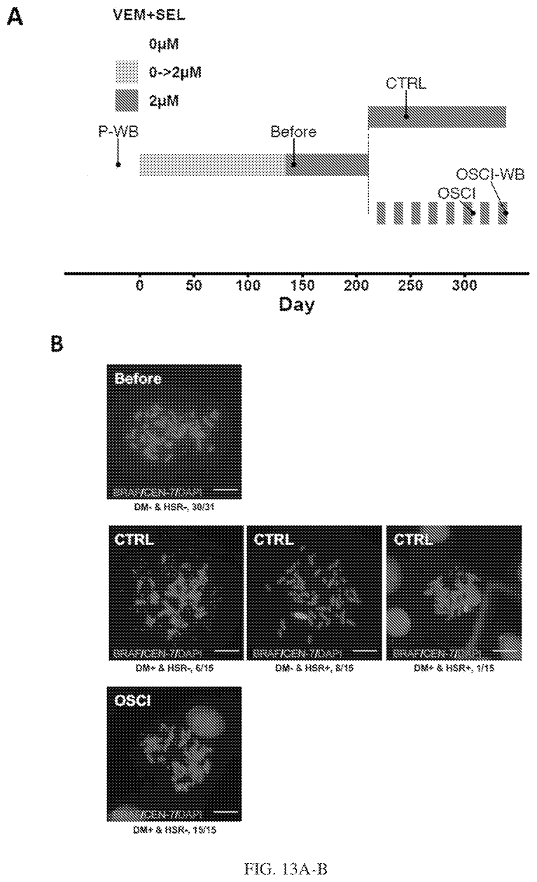

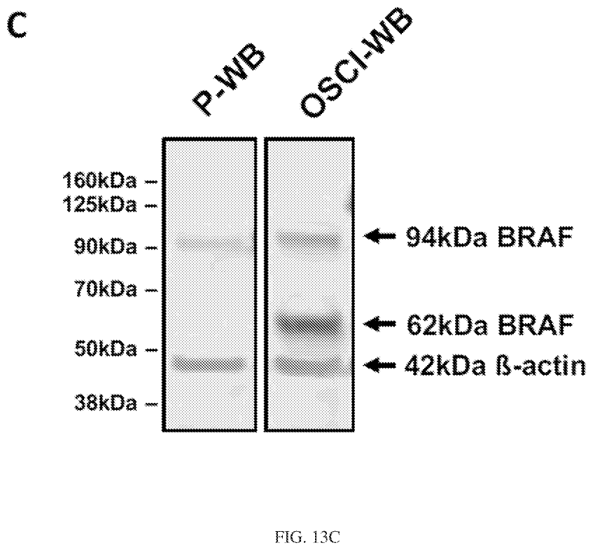

The observation that in the M249-VSR system DMs will integrate into chromosomes as HSRs upon continuous culture at a constant drug dose, suggests that DM+ cells have a fitness disadvantage compared to HSR+ cells in these conditions. However, DMs are often observed in tumor samples, and thus may have a fitness advantage in other situations. To test this hypothesis, the inventors aimed to identify a scenario in which DMs would have a fitness advantage. DMs are known to segregate asymmetrically during cell division (13-16), so the inventors tested whether an oscillating drug dose would give DM+ cells increased fitness, arguably through increased heterogeneity of the population. The inventors designed an experiment in which the inventors turned the double-drug doses on and off in a cycle of 8 days (FIG. 3A, EXP1-2). DMs were indeed retained without a switch to an HSR state for a longer period of time compared to the steady dose scenario (FIG. 3A-C, FIX5 and FIG. 13A-B). However, the number of DMs did decrease in these cells, suggesting another MAPK inhibitor resistance mechanism had emerged in these cells. The inventors found that these cells express the shorter BRAF splice isoform associated with acquired resistance whereas the bulk HSR cells do not show this isoform (34) (FIGS. 1D and 13C). Hence, in response to the altered fitness challenge of a regularly changing environment, the emerging cells retained DMs longer than cells experiencing constant drug dose, and the non-constant conditions furthermore resulted in the expression of an additional resistance-associated BRAF isoform that likely reduced the overall BRAF expression requirement, and thus led to lower DM copy numbers.

### 4. MAPKi-Induced DMs and HSRs Display Dynamic Plasticity Upon Changes in Drug Dose

Next, the inventors focused on studying the plasticity of DMs and HSRs in M249-VSR cells. As a foundation for this analysis, the inventors first examined whether HSRs were the final stable form of amplicons for cells kept under constant drug dose by checking their karyotypes after a few additional months. The inventors found that most cells still harbored HSRs with similar amplicon length and BRAF copy number (FIG. 3A-D, EXP1). This stable result provides the reference control for comparison to other cases with drug dose manipulation.

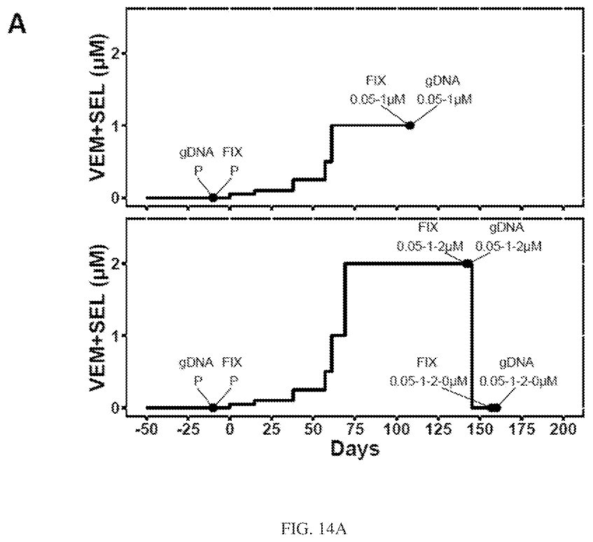

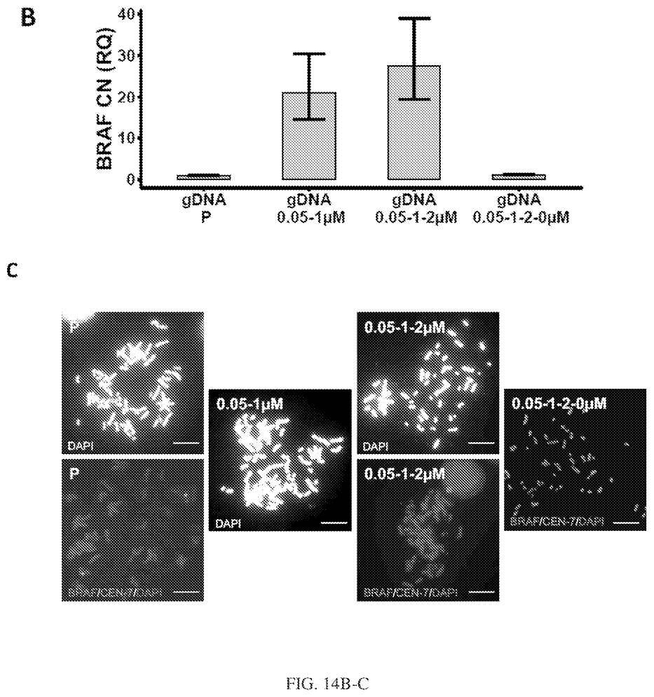

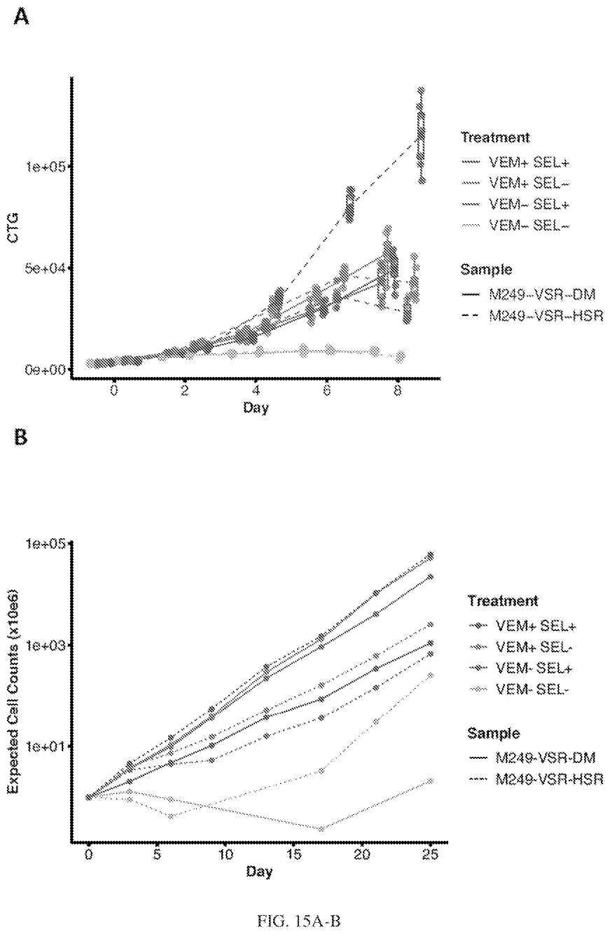

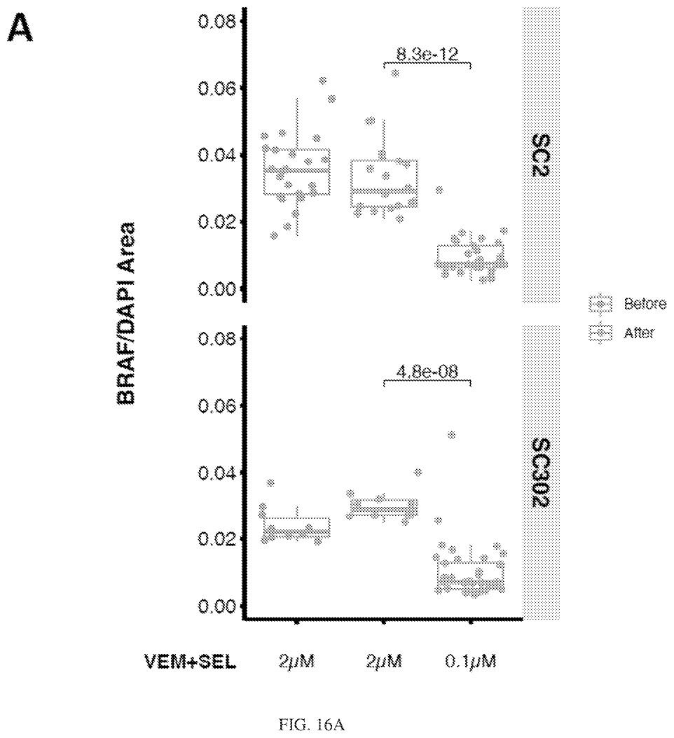

To evaluate if the DM to HSR trajectory observed under constant inhibitor dose could be affected by changes in dosing, the inventors next either decreased or elevated the double-drug concentration being applied to DM+ or HSR+ cells. Previous studies have examined the potential of using drug holidays to eliminate drug-addicted cells (41,43,46,47), thus sparking the inventors' interest in studying the effect of this approach on DMs and HSRs. To investigate this, the inventors withdrew VEM+SEL treatment from M249-VSR-DM and -HSR cells. In the DM+ case, when doses were acutely brought down from 2 μM to 0 μM, all DMs were eliminated based on FISH analysis with the fastest change observed in 12 days. qPCR results showed that the copy number of BRAF was reduced drastically (FIG. 3A-D, EXP3; FIG. 14A-C). The inventors also performed experiments in which only one of the two drugs was withdrawn. Upon single withdrawal of either drug, the inventors saw substantial, but less-complete reduction in DM copy number (FIG. 3E-F) in comparison to the double drug removal. This result supports that in the M249 system, the combined effect of BRAFi and MEKi is a notably stronger amplification selective force than the effect of the single inhibitors. With single drug withdrawal there were minimal effects on cell viability and growth rates. In contrast to the DM case, HSR cells upon single drug withdrawal show reduced viability, supporting HSRs as a less plastic FA mode in this context (FIG. 15A-B).

A prompt reversion of BRAF copy number to the parental state in three weeks also occurred in HSR cells upon full removal of the dual inhibitors (FIG. 3A-D, EXP4). Notably, there was not a substantial difference between the recovery time of DM and HSR cells in these double drug wash-out experiments (FIG. 15A-B). These results motivated additional experiments to test the plasticity of the HSR FA mode. The inventors next repeated the dose decrease experiment above using the bulk population in its HSR+ state but did not perform a complete withdrawal (FIG. 3A-C, EXP5: 2 μM to 0.1 μM). In this experiment, the bulk population demonstrated a substantial shortening of the typical HSR length, but HSRs were still detectable. Using this new sub-population, the inventors further explored the cellular genomic plasticity by subsequently reinstating the 2 μM double drug dose. The cells regained resistance in less than a month, and most cells again presented with the longer form of HSRs (FIG. 3A-C, EXP5). During the interval of drug reduction and increase, BRAF DNA copy number also decreased following the 2 μM to 0.1 μM transition, and re-increased following the 0.1 μM to 2 μM transition accordingly (FIG. 3D, EXP5).

The inventors also reinstated a 2 μM drug dose on the bulk population of cells that had drug withdrawal (0 μM) occur while they were in the DM+ state (FIG. 3A, EXP3). In this case, it took about 4 months for the cells to re-develop resistance to VEM+SEL, similar to the time required for the initial establishment of resistance in the parental cells. In this experiment, the melanoma cells demonstrated an additional variation in that upon becoming resistant they typically harbored two or three separate, shorter HSRs on different chromosomes (FIG. 3B-C, EXP3). None of the cells presented with a single larger HSR. This treatment course thus further revealed the plasticity of genomic options available for adjusting to changes in selection pressures.

Notably, the inventors could generalize a subset of these copy number plasticity findings to other melanoma samples and other amplified genes under MAPKi challenge. BRAF^{V600}E cell lines A375 and Mel888 harbor BRAF HSRs upon acquiring dabrafenib (BRAFi) plus trametinib (MEKi) resistance (DTR) (35,43), and harbor shortened HSRs after dose reduction (FIG. 3G-H). The inventors also characterized NRAS^{Q61R} or BRAF^{S365L} melanoma patient-derived xenograft (PDX) models. Upon establishment of MEKi trametinib resistance, these models acquire RAF1 (CRAF) amplifications in extra- or intra-chromosomal modes, respectively, and the RAF1 amplifications decreased after drug withdrawal (FIG. 3I-L). To the inventors' knowledge, this is the first documented example involving RAF1 DMs mediating MAPKi resistance.

### 5. Single-Cell-Derived Clones Demonstrate a De Novo Component to the Plasticity of BRAF DM and HSR Focal Amplifications

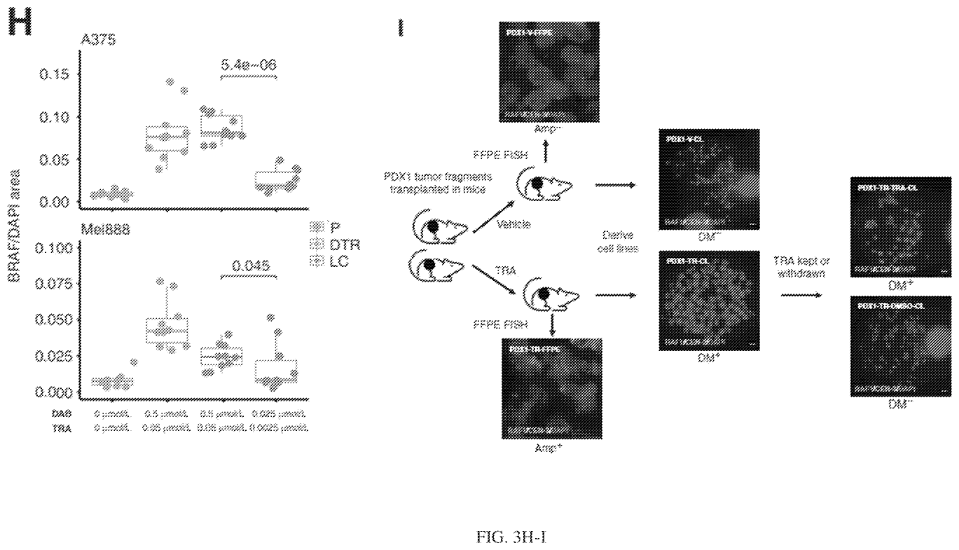

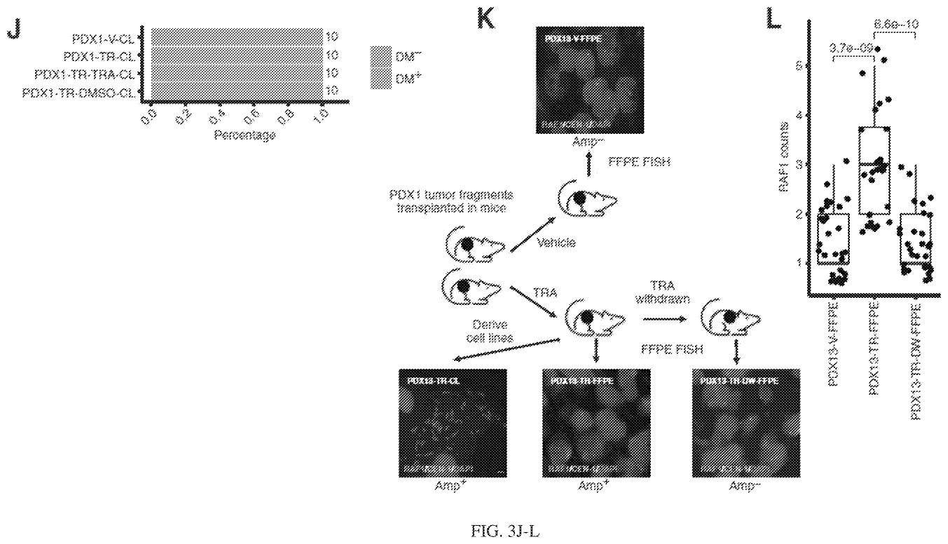

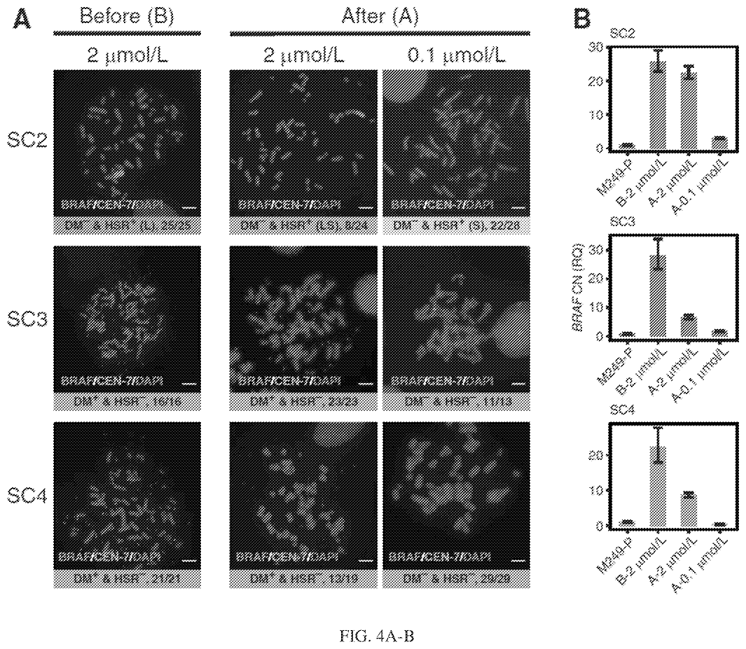

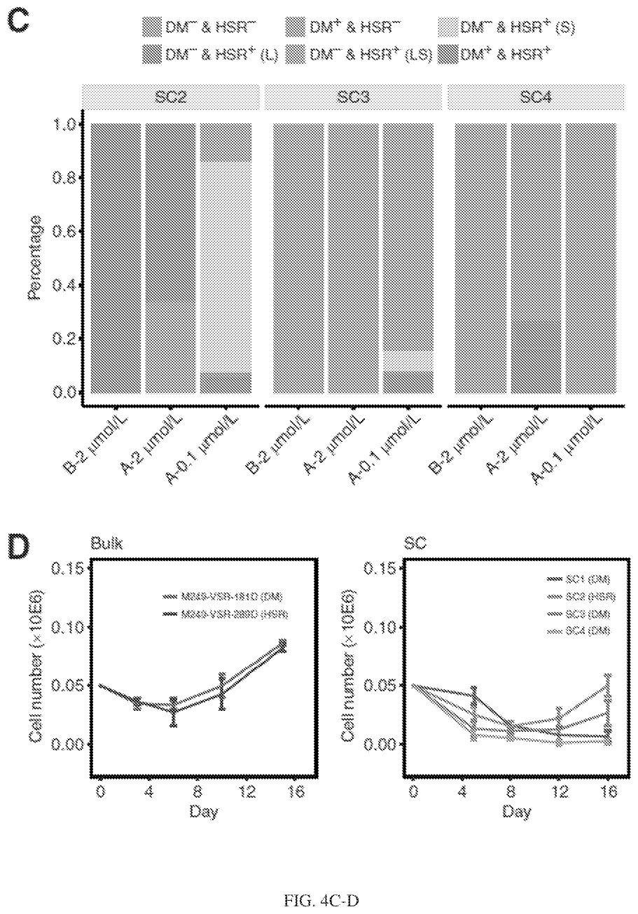

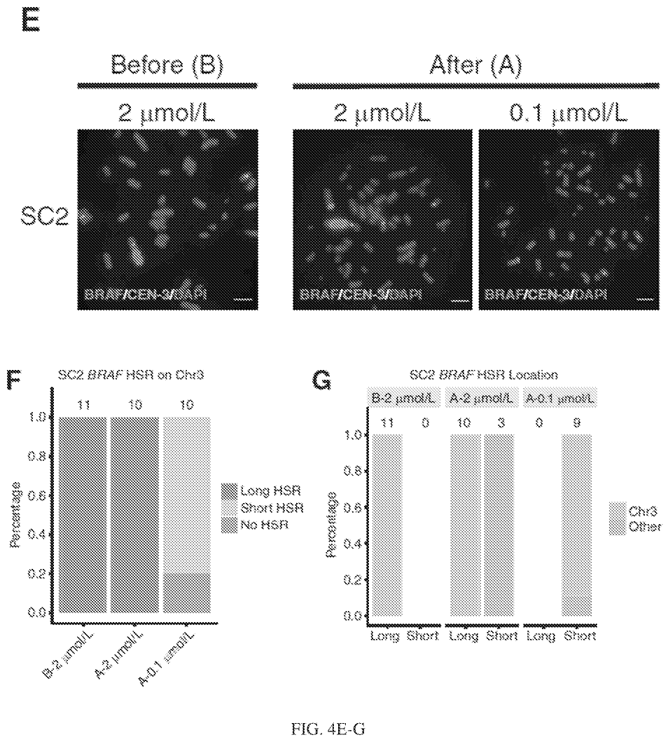

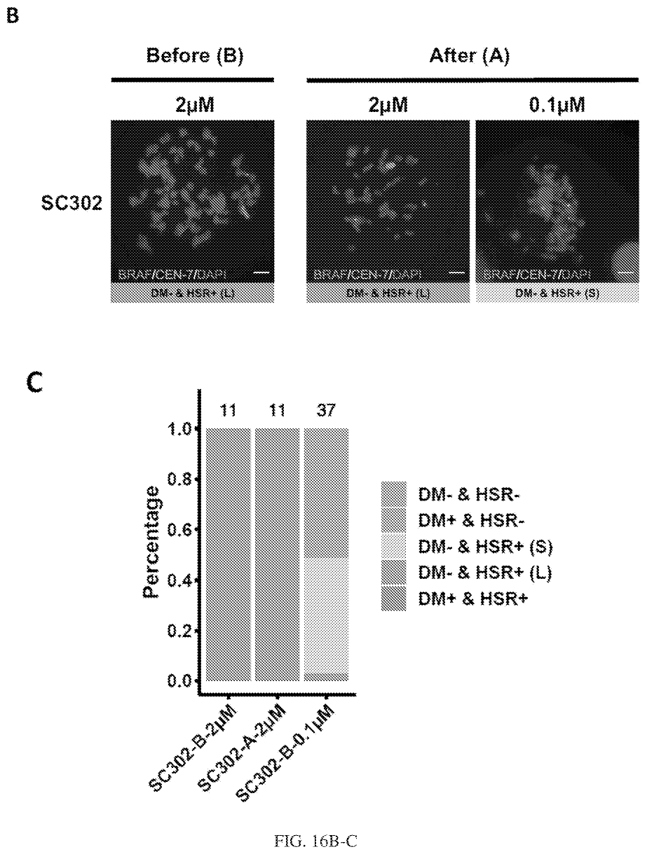

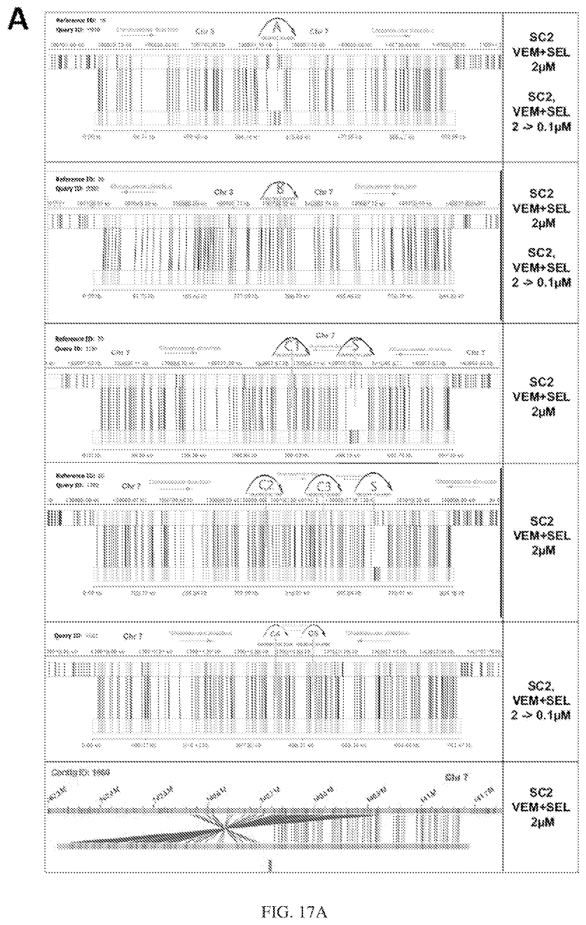

The dose decrease and increase results above could be explained by selection for residual BRAF copy number-low or -high cells in the respective populations. To investigate cellular plasticity to dramatic drug reduction in a more homogeneous population, the inventors turned to the single cell-derived DM+ or HSR+ clones. In these experiments the inventors lowered the VEM+SEL double dose from 2 μM to 0.1 μM using clones SC2 (HSR), SC302 (HSR), SC3 (DM) and SC4 (DM). For controls, the inventors kept the dual dose at 2 μM. In the post-drug-decrease populations, SC2 and SC302 showed reduced length of HSRs, and SC3 and SC4 showed reduced number of DMs. All cases were accompanied by a substantial decrease in BRAF copy number (FIG. 4A-C, FIG. 16A-C). Another characteristic that indicates the plasticity of HSR-harboring cells is in some regards comparable to that of the DM case is that the recovery times upon dose withdrawal for DM+ or HSR+ cells, either bulk or as SCs, were not substantially different (FIG. 4D).

While DM plasticity can be explained by uneven segregation (13-16), HSR plasticity, especially such rapid change in one month, is uncommon during dose challenging—purportedly due to the stability provided by chromosomal integration (48-50). The inventors thus further analyzed the structural data related to the long to short HSR transition upon dose reduction. To reduce heterogeneity, the inventors used the HSR+SC2 clone, with its initial long HSR on chromosome 3 (FIG. 2E). In most cells from clone SC2, the post-dose-reduction, short HSR remained located on the same chromosome based on FISH staining. Nevertheless, the aforementioned small HSRs (via HSR duplication) on different chromosomes were either not yet present, or not favored by selection upon dose reduction, in comparison to the shortening of the Chr3 HSR. Taken together, these results demonstrate the de novo evolution of the clonal long HSR both during constant drug dose (HSR duplication), as well as upon dose reduction (HSR shortening) (FIG. 4E-G).

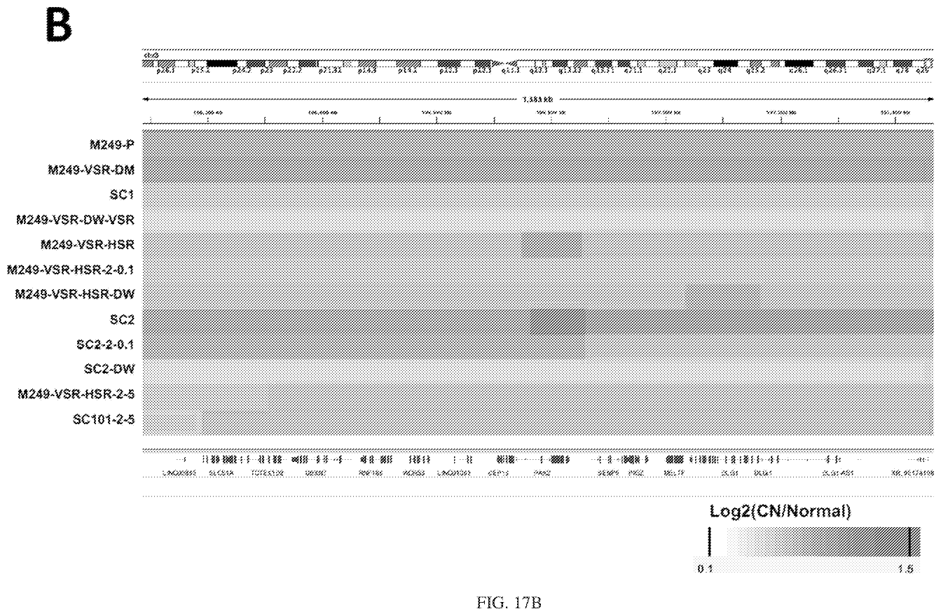

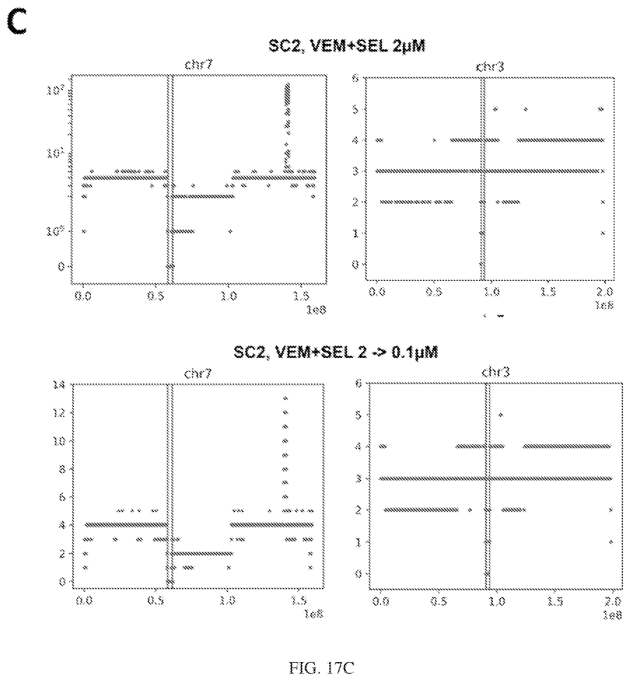

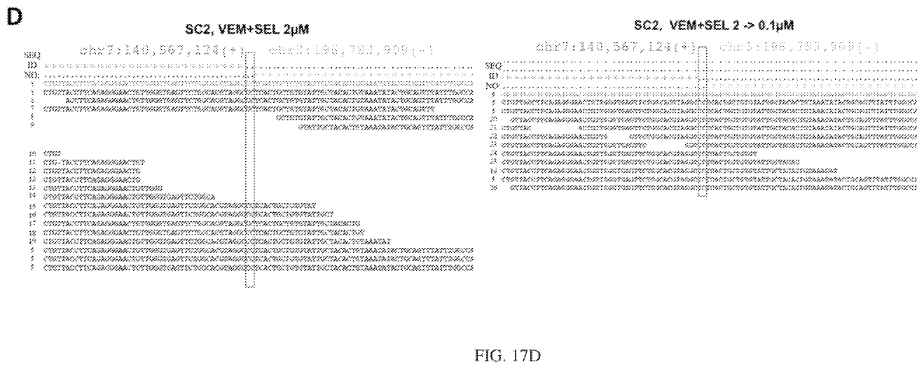

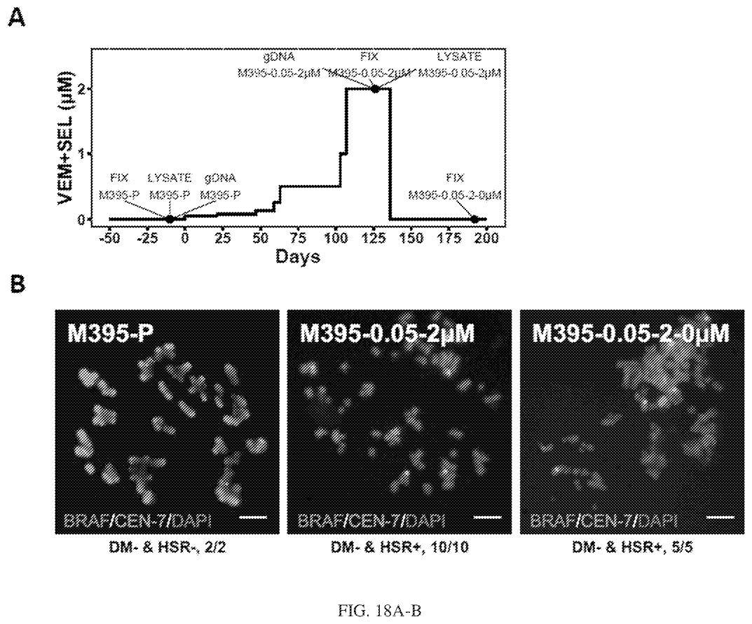

The longer fragment lengths of optical mapping (OM) aided in the investigation of the structure of such plastic HSRs. The OM data indicates that the BRAF HSR structure in SC2 is complex and involves duplications (primarily head-to-tail) and some inversions. The HSR was integrated at the PAK2 gene locus near the telomere of chr3 (FIGS. 4H-I, 10D, 17A and Supplementary Table S1), in line with previous findings that telomeres and telomere-proximal sites are more frequent locations for integration (22,28). The PAK2 locus was duplicated, and the integration occurred between the two PAK2 copies (FIG. 4H). Increases in the copy number based on both OM and WGS data support such PAK2 duplication at the site of integration (FIG. 17B-C). WGS additionally demonstrates that the breakpoint junction between chromosome 3 and 7 contains a two-nucleotide non-templated insertion, which supports a potential role for NHEJ (51) (FIG. 17D) and is line with the aforementioned finding about ecDNA integration dependency on DNA-PK (FIG. 2E).

After VEM+SEL dose reduction, the number of BRAF amplicon repeats decreased, along with the creation of new breakpoints and the generation of a more heterogenous population. However, the integration junction next to PAK2 was preserved in a subset of the cell population (FIG. 4H-I and Supplementary Table S1). Overall, the combined OM and WGS data support a model of in situ excision of BRAF amplicon repeats (from within the HSR, not from the ends), potentially through error and repair mechanisms, in the long to short HSR transitions that are observed upon dose reduction.

The inventors expanded such finding of DM and HSR plasticity to MEKi-resistant subclones from a human NRAS^{MUT} melanoma cell line (M245), involving different amplified oncogenes. Clone 3 (C3) of M245 cells harbor RAF1 amplification as DM upon becoming resistance to trametinib, while clone 5 (C5) harbor NRAS amplification as an HSR. Drug withdrawal caused copy number decrease in both cases: reducing RAF1 DM number and shortening the NRAS HSR (FIG. 4J-K). The RAF1 and NRAS amplification events have been shown previously to mediate resistance to MEKi in these cell lines (52).

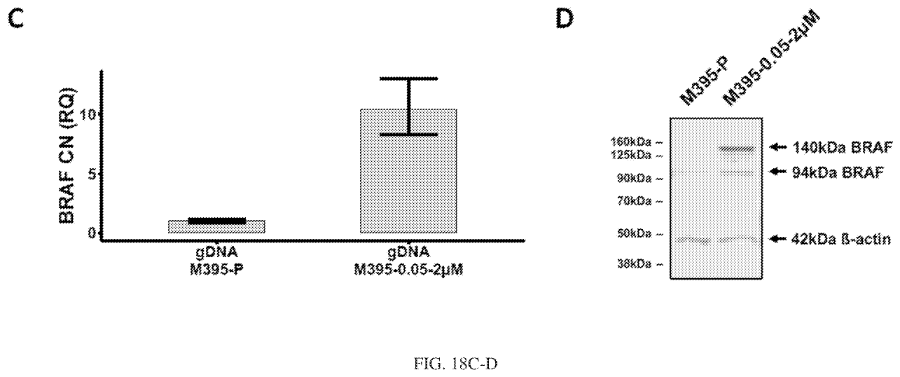

While these bulk and single cell clone experiments demonstrate the plasticity of HSRs, the inventors also identified a melanoma cell line with HSR-based focal amplification that does not show shorten or lost HSR upon BRAFi+MEKi removal (FIG. 18A-D), which is similar to some previous observations and conclusions about HSR stability (48,49).

6. Karyotypic Shift from HSRs to DMs Carrying BRAF Kinase Domain Duplications Upon Double-Drug Dose Increase

To further investigate HSR plasticity, the inventors increased the double MAPKi doses applied to the bulk M249-VSR cells at a timepoint when they were predominantly HSR+. Interestingly, this treatment converted the population of predominantly HSR+ cells to predominantly DM+ cells (FIG. 3A-3C, EXP6). Contrary to the expectation that in the higher drug dose the cells would have higher levels of BRAF DNA copy number, the inventors found that the copy number had decreased (FIG. 3D, EXP6).

To investigate this change further, the inventors repeated the experiment using bulk M249-VSR-HSR cells at various time points over the entire HSR-harboring period, roughly 260 days onwards from the beginning of resistance development (FIG. 5A). Four out of five dose-increase experiments resulted in changes of FA types from HSRs to DMs (VS5-1, VS5-2, VS5-3, VS5-4 and VS5-6 (5 sampling points in total)). One out of five resulted in cells that were DM− and HSR− VS5-5) (FIG. 5B-C). Notably, the inventors found that the five DM+5 μM-resistant samples all expressed a BRAF protein variant with a molecular weight of approximately 140 kDa (FIG. 5D). Four of the five DM+5 μM-resistant samples also expressed the 62 kD variant of BRAF, the BRAF inhibitor-resistant splice variant observed in the oscillating dose experiment above. The 140 kD size matches a previously reported BRAF variant with a kinase domain duplication (KDD) that leads to BRAF inhibitor resistance (35,53,54). Based on RT-qPCR using primer pair that spans the BRAF exon 18-10 junction, the inventors discovered that all of the HSR to DM transformed samples carried exon 18-10 junctions, while other cultures, including M249 parental (VSO), M249-HSR cells prior to dose increase (VS2-1, VS2-2, VS2-3 and VS2-4) and M249-HSR cells that showed DM− & HSR-post dose increase (VS5-5), contained none or only a minimal amount of such junctions (FIG. 5E-F). The close to unity ratio of 18-10 to 9-10 junctions supports that each DM unit contains one KDD region in the KDD expressing sublines.

The inventors next investigated whether the KDD was developed due to selection of an existing subpopulation or de novo kinase domain duplication after the 2 to 5 μM dose increase. Under constant 2 μM dose VEM+SEL, the M249-VSR resistant cells were initially primarily DM+ & HSR− (circa day 150), turned primarily DM− & HSR+ with time (circa day 260), and then with additional time reacquired a small percentage of DM+ & HSR− cells (450 days and onwards, FIG. 5A-C). Their late timepoint DM+ & HSR− fractions were 2/46 (4.4%, VS2-3) and 3/30 (10%, VS2-4). This expanding DM+ population could have been the source of KDD that expanded post drug dose increase to 5 μM.

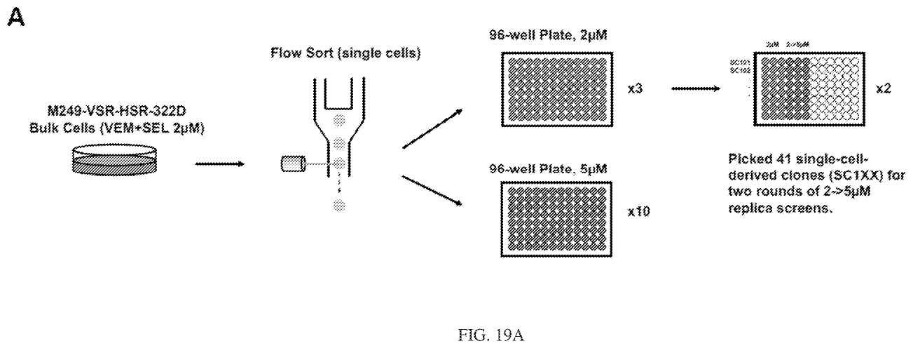

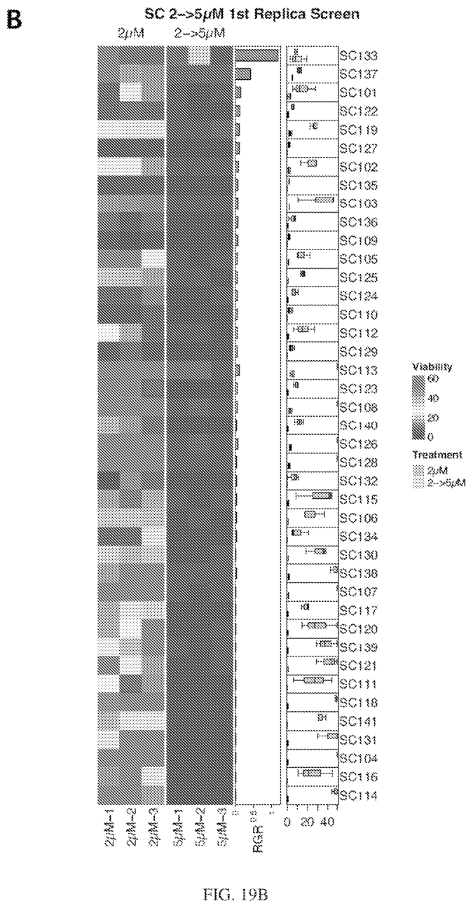

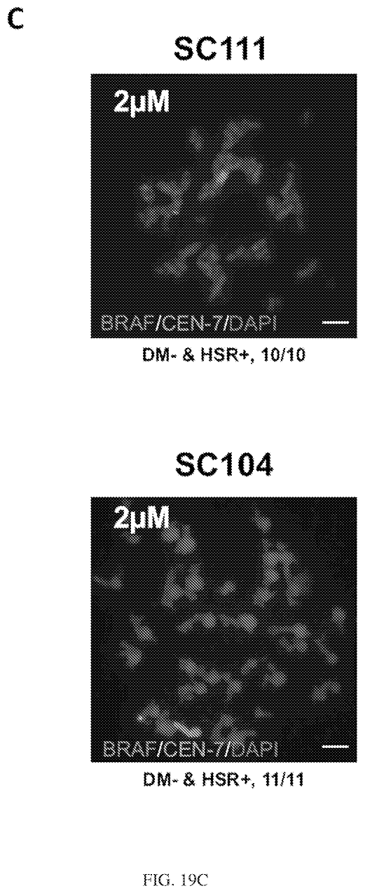

To further test if rare DM+ cells were present at earlier times below the level of detection by bulk FISH analysis, the inventors used both single cell sorting and a replica plating approach. First, cells from the earlier-stage M249-VSR-HSR bulk population (322 days) were single cell sorted. This collection of single cell clones was then either treated at the original 2 μM dose or at an elevated 5 μM dose of VEM+SEL (FIG. 19A). The inventors found that 3.2% of the single cell clones could grow under 5 μM in a similar manner compared to their counterparts at 2 μM (large colonies, FIG. 5G).

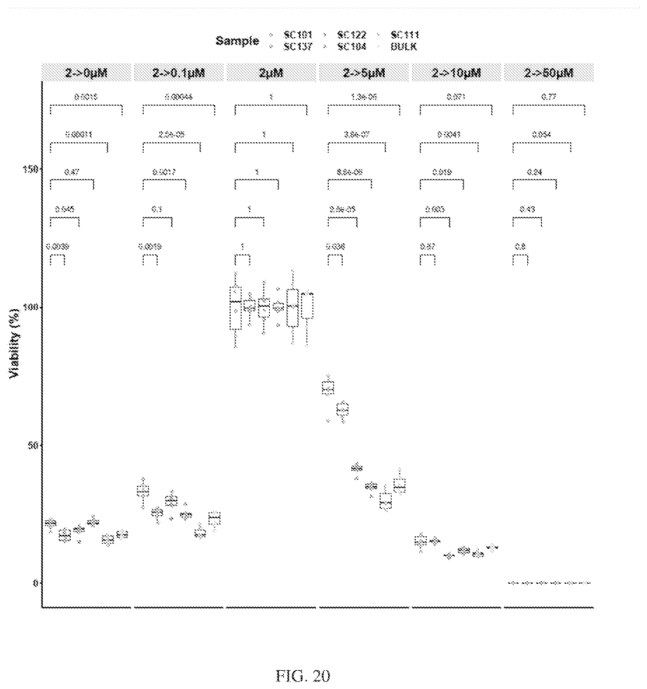

Next the inventors added a replica plating step. Forty-one single-cell clones derived at 2 μM were replica plated, and then treated in parallel at either the original 2 μM dose or the elevated 5 μM dose (FIG. 19A). After two rounds of screening, the clone with the highest relative growth rate (SC101) was revealed to be DM+ & HSR− both before and after the dose increase, with no observed cellular heterogeneity of FA modes (FIG. 5H-I). The second fastest clone (SC137) started with a 10% DM+ & HSR− population, but finished at 100% DM+ & HSR− at the end of the replica plating (FIG. 5H-I). Four other randomly selected SCs from either the near-top of the relative growth rate-sorted list and the bottom of the list displayed no DM+& HSR− karyotype (SC122, SC124, SC111, SC106, FIG. 5H-I, 19B-C). The two fastest SCs, SC101 and SC137, did harbor BRAF KDD on their DMs based on immunoblot analysis (FIG. 5J). In a second quantitative viability assay, the SC101 and SC137 KDD+ SCs again demonstrated the best ability to tolerate drug dose increases (FIG. 20A). This replica screening result supports that the cells harboring the BRAF KDD containing DMs pre-existed in the bulk population prior to increases in the dual MAPKi dose. These cells were starting to expand in the 2 μM drug condition with a relative fitness slightly higher than other cells, but the increase in drug dose sharply increased such fitness advantage. Using a barcode-based clone tracing system (ClonTracer) (55) to keep track of the subpopulations in the bulk M249-VSR-HSR cells (from day 318), the inventors observed that even under the constant 2 μM drug dose and at such later timepoints, certain cells did expand faster than others (FIG. 5K-L), indicating the bulk population of cells continues to evolve in regards to its subpopulation distributions.

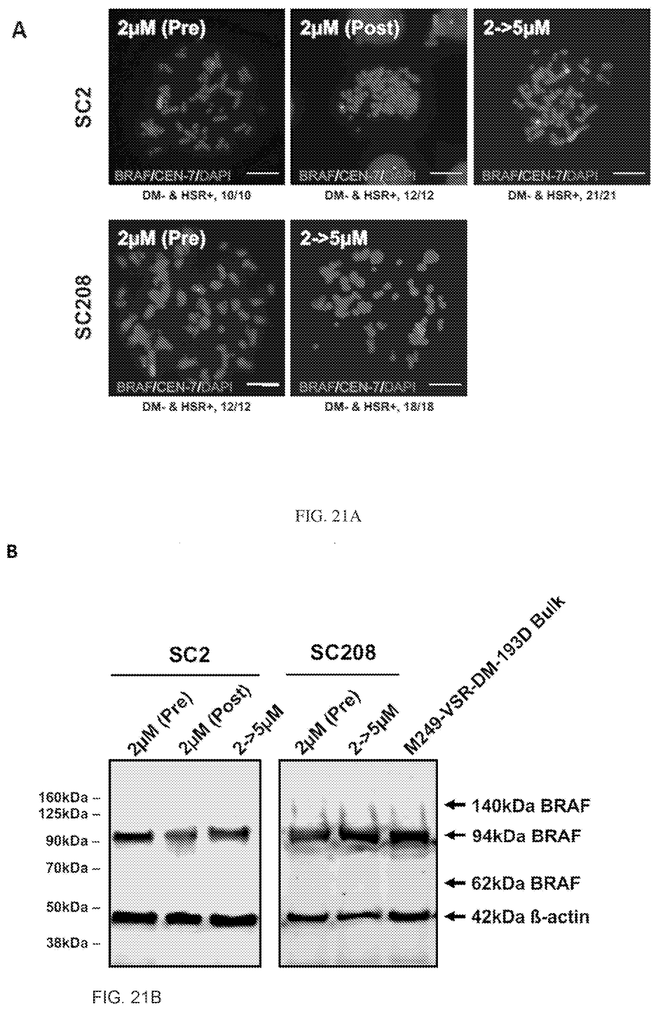

Interestingly, the inventors did not observe de novo generation of DMs, either KDD-bearing or not, from BRAF DM− & HSR+ SCs upon performing dual drug (VEM+SEL) escalation (FIG. 21A-B). Successful HSR to DM transitions have been demonstrated in different cell types, involving different genes, with corresponding different drug regimens. These reported cases typically involve the creation of fragile sites or chromothripsis on HSRs (22,26).

### 7. Cells Preserve BRAF Amplicon Boundaries Under Various Dose Challenges

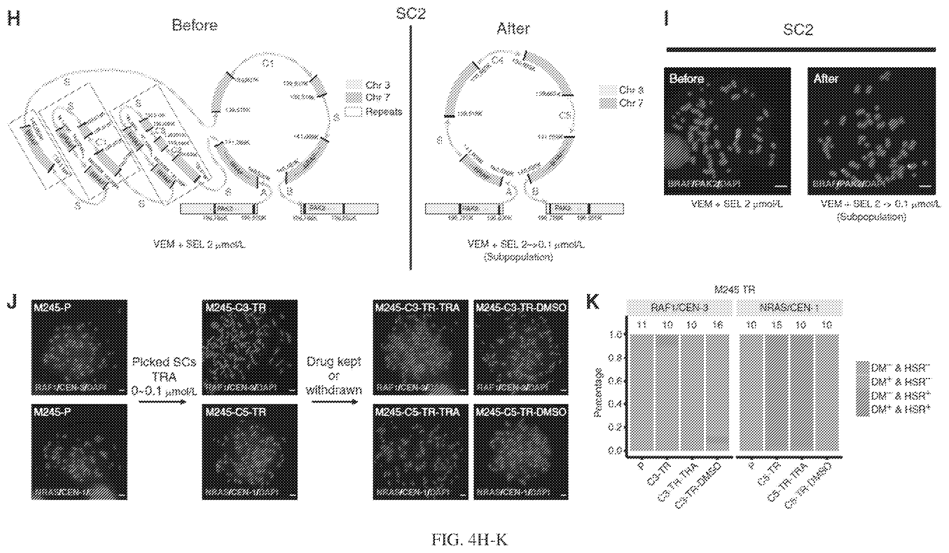

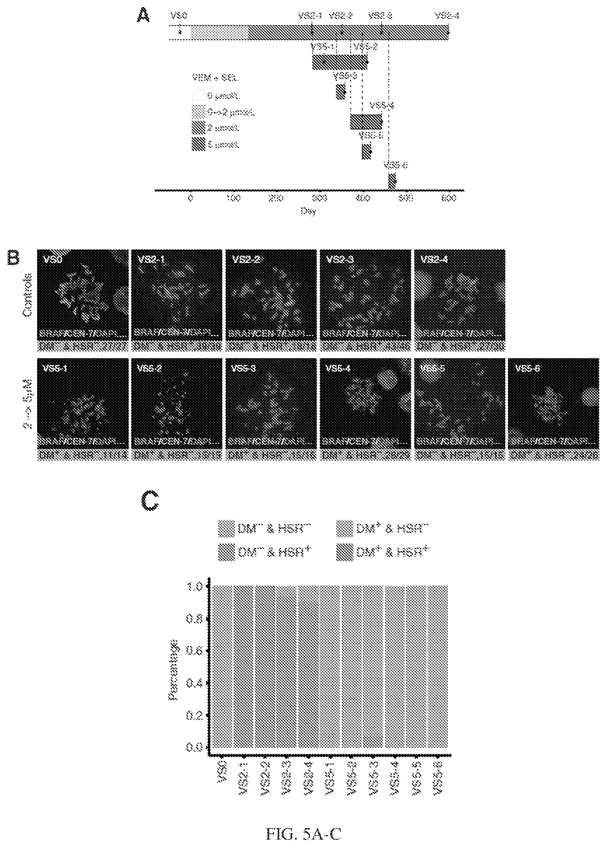

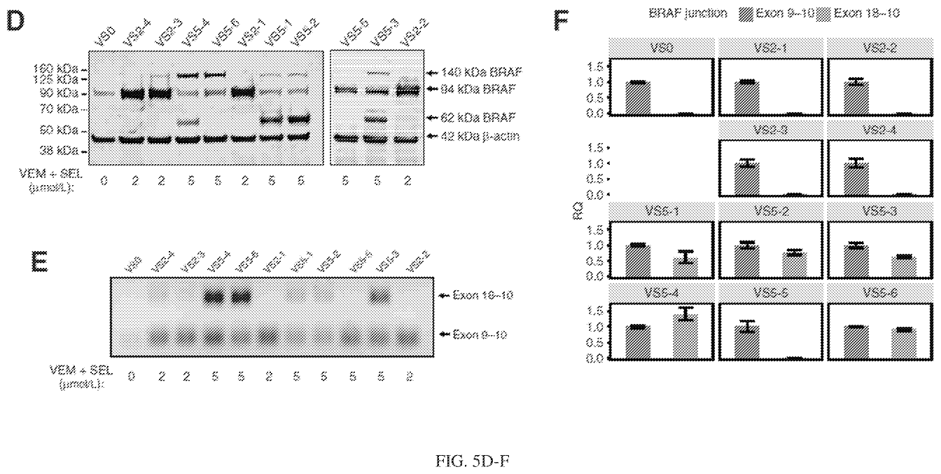

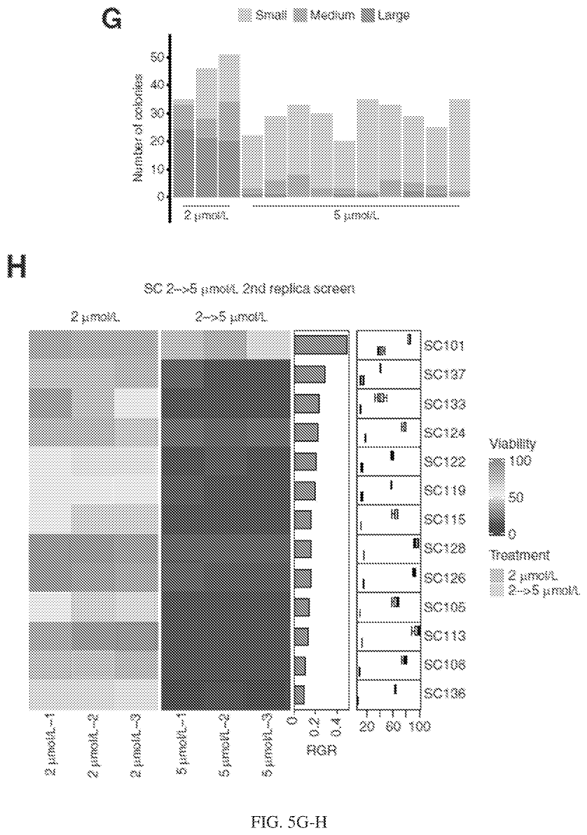

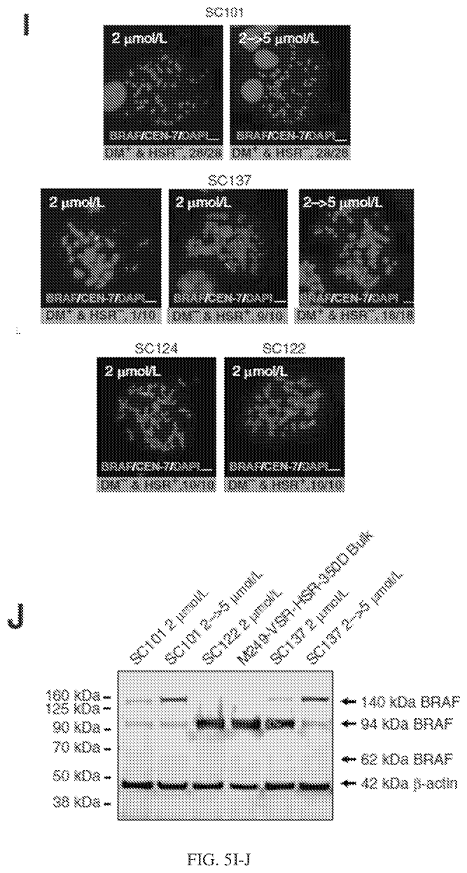

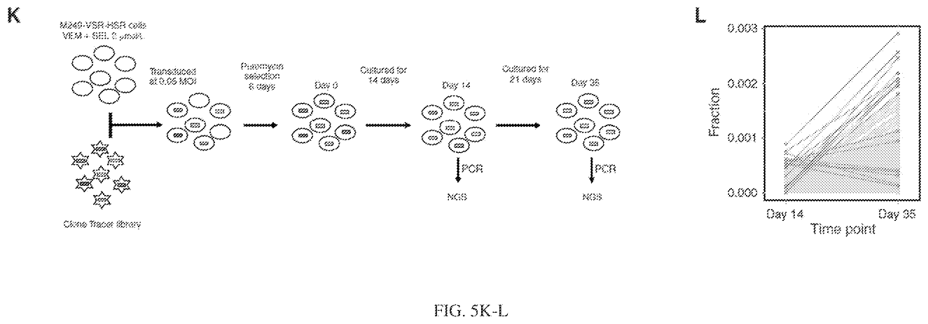

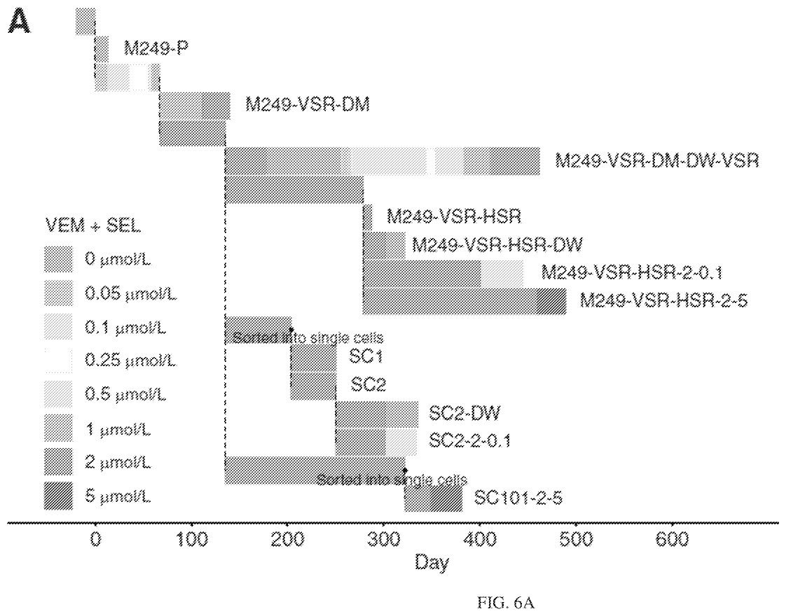

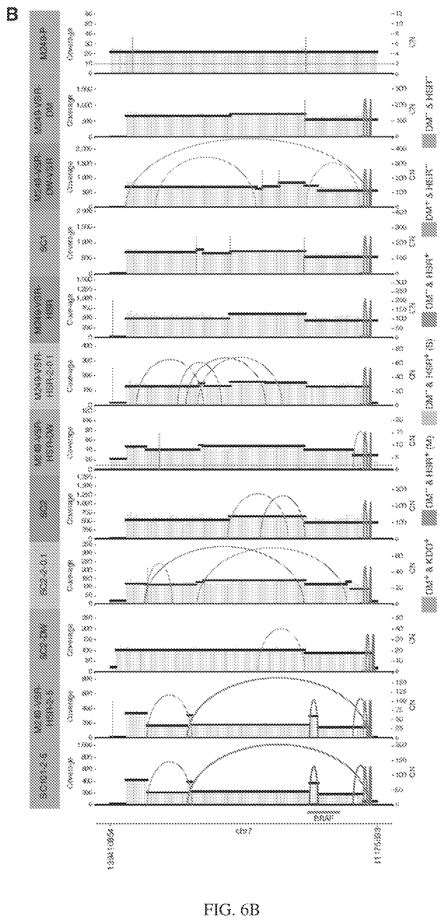

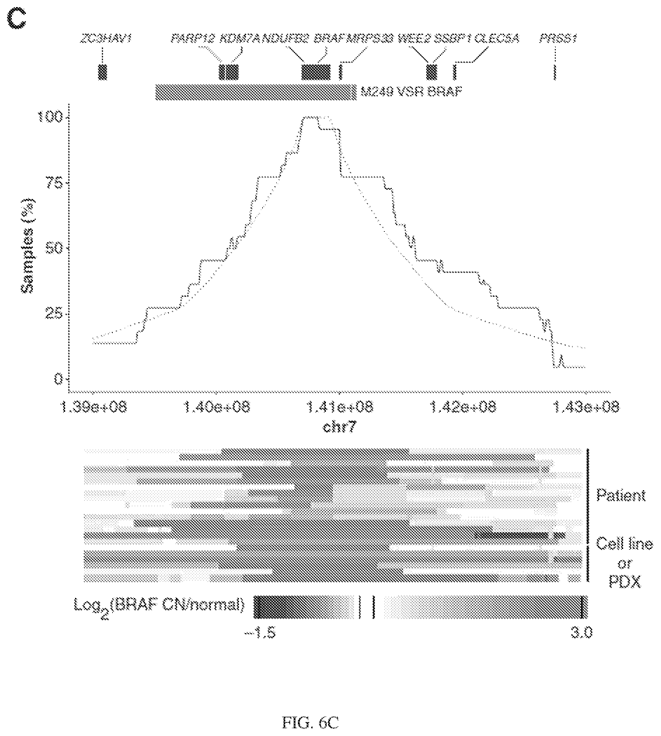

After learning the plasticities of BRAF-containing DMs and HSRs in response to dose perturbations, the inventors next investigated if amplicon boundaries and junctions changed during these processes. The inventors performed structural variant (SV) analysis on the M249 samples collected after various dose challenges (FIG. 6A) and found that genomic amplification boundaries do not differ substantially regardless of their FA mode, amplicon number, sub-cloning status, nor presence or absence of the KDD selection, supporting a single initial amplicon origin (FIG. 6B). This conclusion is further corroborated by the conserved junctions connecting amplicons in bulk and SC DM+ and HSR+ sublines cultured at full drug dose (Supplementary Table S2). New junctions were generated during dose decreases and KDD formation, but these alterations did not alter the overall amplification coordinates (FIG. 6B).

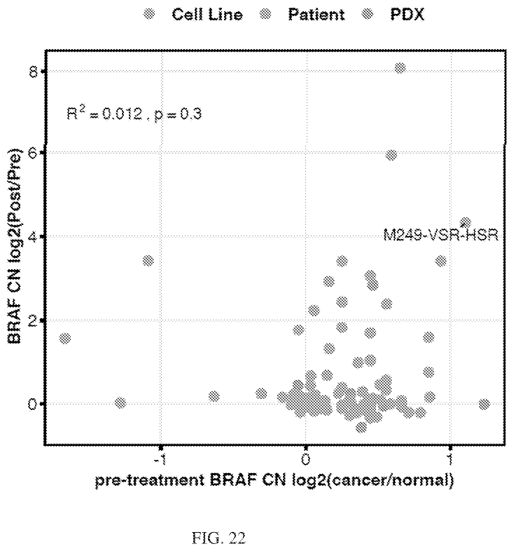

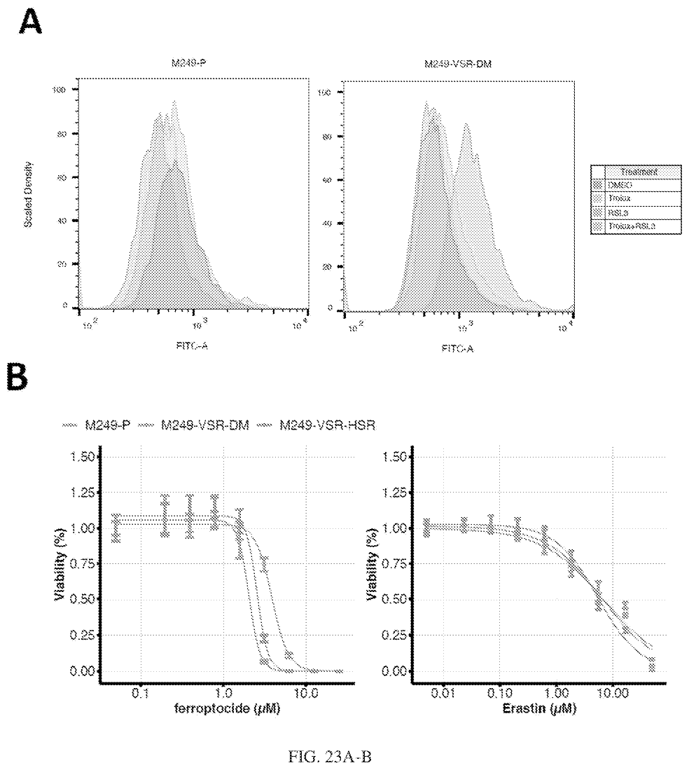

High BRAF amplification upon acquired MAPKi-acquired resistance is observed in the clinic with at least 16 patient-based cases reported in the literature, in PDX models (35), and in other cell line models (data not shown). In amplicon boundary analysis of the cohort, the inventors found the M249 FA boundary to be consistent with the range of all observed boundaries. Furthermore, the inventors did not find evidence for inclusion of co-amplification loci adjacent to BRAF (FIG. 6C). This observation is in line with some frequently amplified oncogenes around which the ecDNA breakpoints distribute randomly (30), and is distinct from MYCN and EGFR amplification in other tumor types that have been shown to involve co-amplification of adjacent enhancers (11,12). Looking across the cohort, the inventors did not observe a relationship between pre-treatment BRAF copy number and the likelihood for BRAF focal amplification post acquirement of resistance (FIG. 22A).

8. Melanoma Cells with BRAF Amplification-Mediated Dual BRAFi+MEKi Resistance Show Increased Sensitivity to Ferroptosis

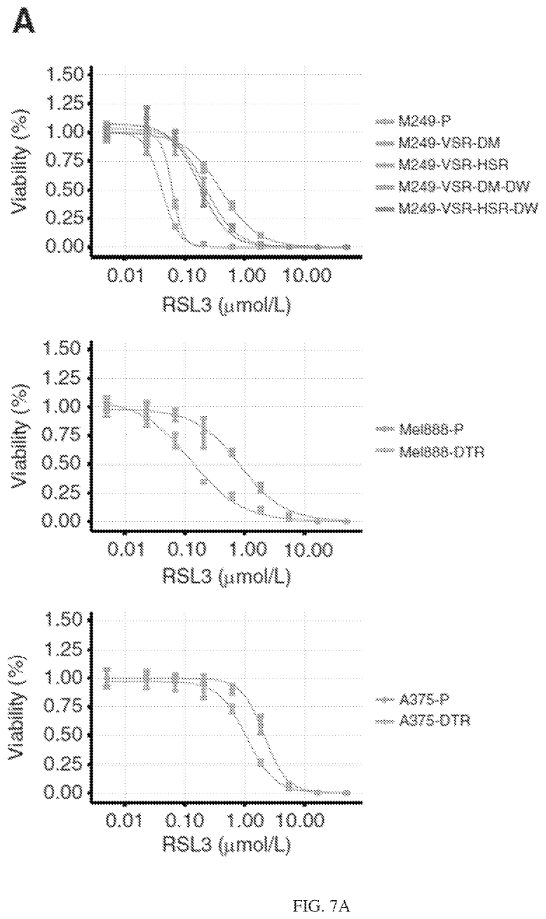

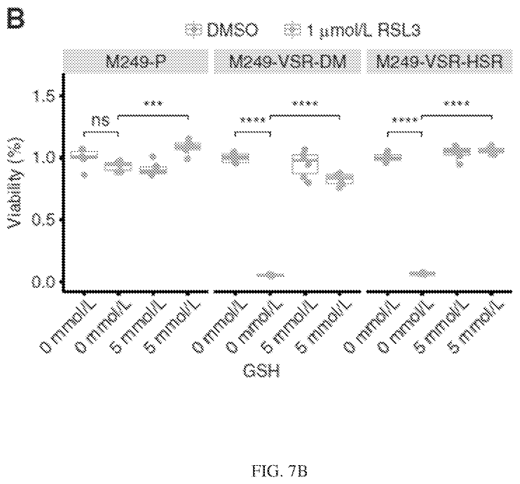

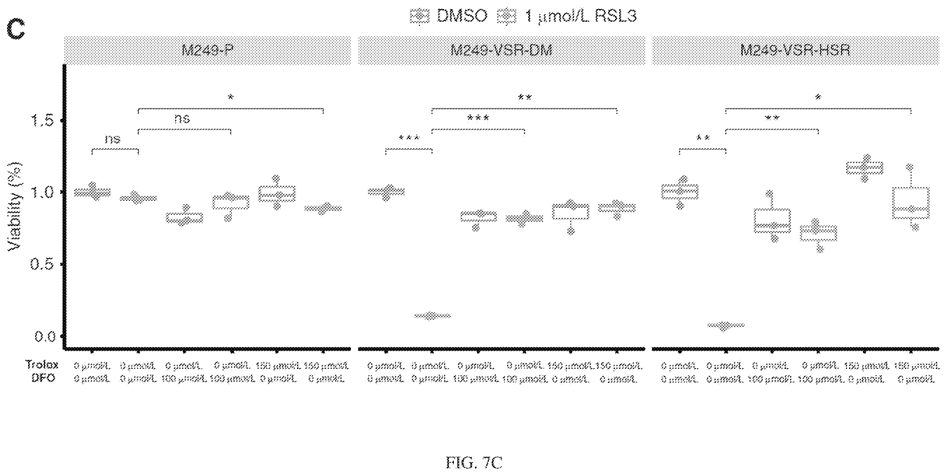

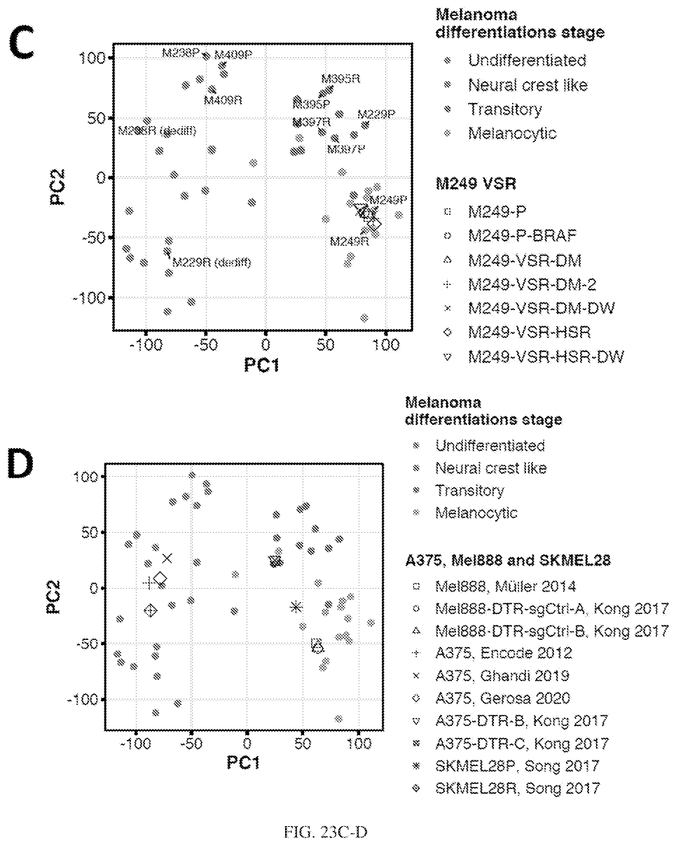

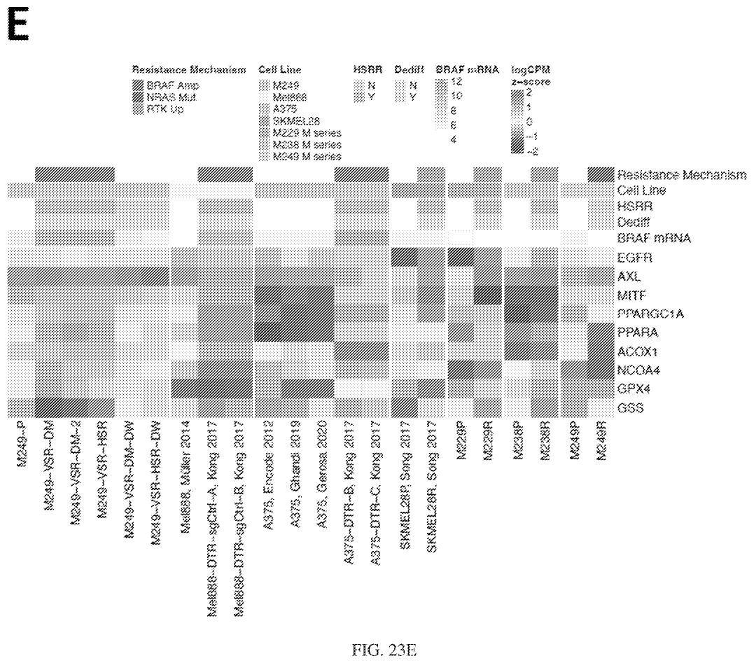

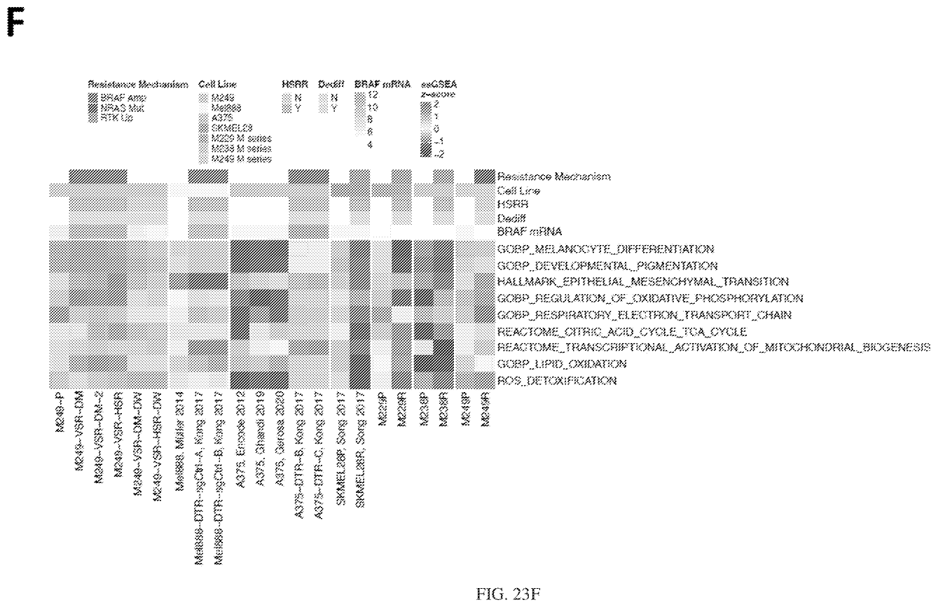

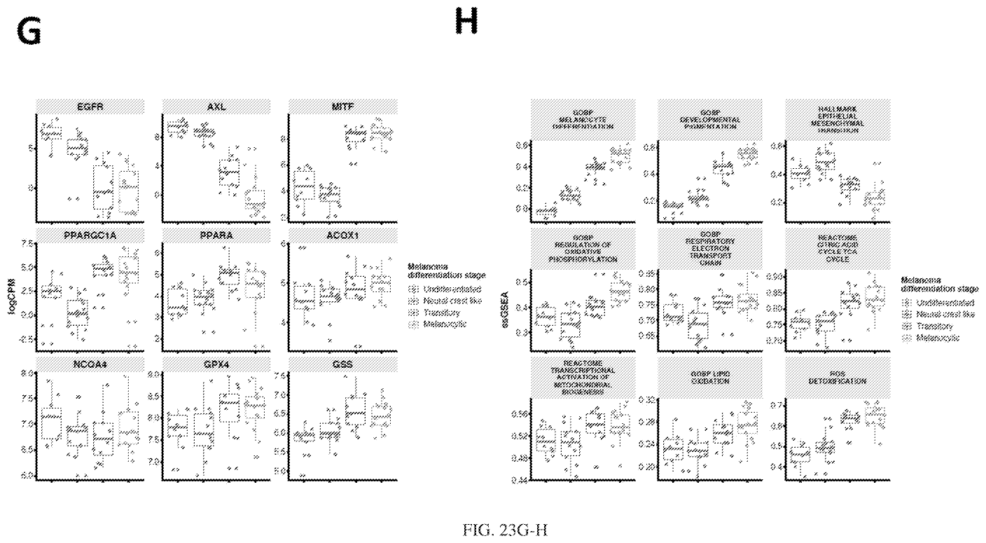

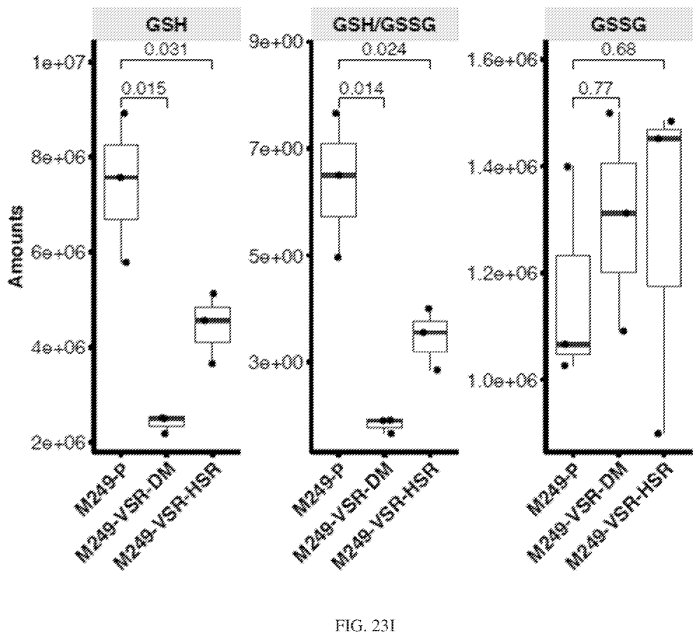

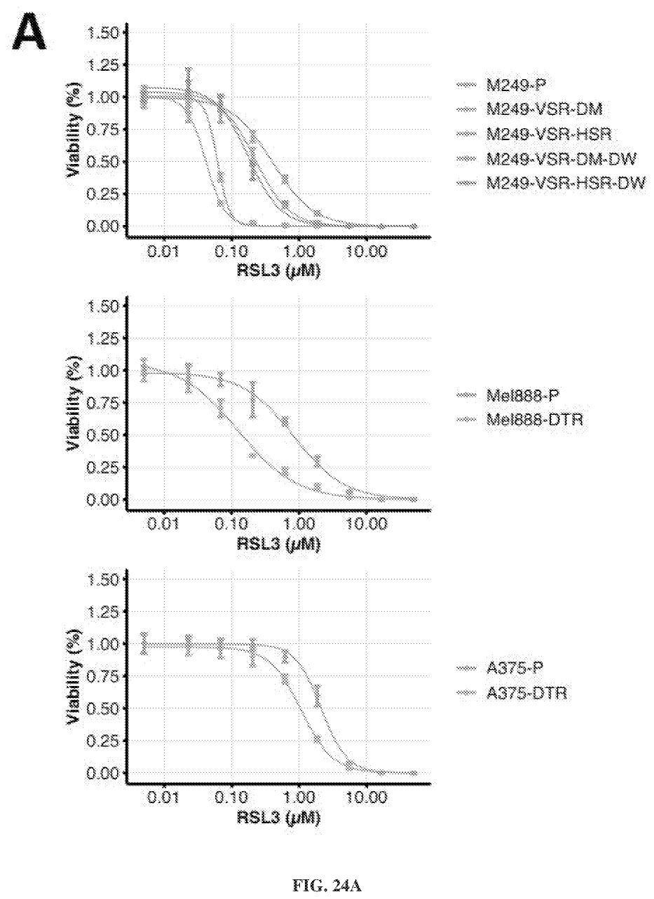

Given the high plasticity of BRAF amplifications in the M249-VSR series, the inventors next investigated cellular vulnerabilities affiliated with amplification in this dual MAPKi context. Previous study revealed a correlation between melanoma differentiation stages and sensitivity to pro-ferroptotic drugs (39). The inventors thus tested if BRAF amplified cells have altered sensitivity to disruption of the repair of oxidized lipids. The inventors tested the sensitivity of the pro-ferroptotic drug RSL3, which targets glutathione peroxidase 4 (GPX4), in the M249-VSR series. The inventors found that both M249-VSR-DM and —HSR are substantially more sensitive to RSL3 compared to M249-P (FIG. 7A). The inventors further found that after drug withdrawal, when the cells have reduced BRAF copy number, M249 cells lose sensitivity reverting to levels closer to the parental cells. Consistently, two additional cases of BRAFi+MEKi-resistance mediated by BRAF amplification, A375-DTR and Mel888-DTR (43), also showed increased RSL3 sensitivity compared to their parentals. The inventors next confirmed that the RSL3 sensitivity in M249-VSR sublines have the expected characteristics of ferroptosis. Namely that the RSL3 sensitivity is reactive oxidative species (ROS)-, lipid ROS-, and iron-dependent, as cell death can be rescued by adding reduced glutathione (GSH), the lipophilic antioxidant Trolox, and the iron chelator deferoxamine (DFO) (FIG. 7B-C). The inventors confirmed an increase in lipid ROS levels in M249-P and M249-VSR cells upon RSL3 treatment, that was protected by the presence of a lipophilic anti-oxidant (FIG. 23A).

The BRAF-amplified M249 dual MAPKi-resistant cells that are sensitive to the GPX4 inhibitor RSL3 were also sensitive to the pro-ferroptotic drug ferroptocide that targets thioredoxin (56), but were not more sensitive to inhibition of the system x_{c}⁻ cystine/glutamate antiporter by Erastin (FIG. 23B). Such differential sensitivity to different upstream components of the glutathione synthesis and ferroptosis pathway have been previously observed (e.g. SKMEL28R (39))

The inventors next investigated why these three cell lines with BRAF amplification- and thus MAPK reactivation-mediated resistance demonstrated higher ferroptosis sensitivity compared to their parental sublines. In previous studies, ferroptosis sensitivity in melanoma is associated with innate or acquired treatment-induced dedifferentiation (39). However, in past work, resistance mediated by reactivation of the MAPK pathway through genomic changes (e.g., via NRAS mutation: M249P/R), does not lead to dedifferentiation and changes in ferroptosis sensitivity (39). The inventors thus analyzed whether the three BRAF-amplified dual MAPKi-resistant cells studied here demonstrated signs of dedifferentiation. Gene expression profiles of RSL3 sensitive M249-VSR (DM and HSR amplification mode), A375-DTR and Me1888-DTR cells (both with HSR mode) do not demonstrate dedifferentiation compared to their parental sublines when their gene expression profiles are projected onto a panel of melanoma lines spanning the full spectrum of differentiation states (39). By contrast, cell lines M229P/R, M238P/R and SKMEL28P/R, which became resistant through upregulation of RTKs (33), did demonstrate dedifferentiation and increased sensitivity to RSL3 (as tested previously (39)) (FIG. 23C-D). Such findings are also supported by the combination of increases in melanocyte differentiation and pigmentation, decreases in mesenchymal gene set scores, and increases in the melanoma differentiation master regulator MITF in the BRAF amplification samples, and the reverse patterns in the RTK upregulation/dedifferentiation cases (FIG. 23E-H).

Upon determination that increased RSL3 sensitivity in these three BRAF amplification cases are not due to dedifferentiation, the inventors then turned their focus to mitochondrial pathways as previous studies have reported that MAPKi resistance can cause melanoma cells to shift their major energy generation program from glycolysis to mitochondrial pathways. This shift then leads to elevated production of ROS and more dependence on ROS detoxifying mechanisms (57-61). Although ROS were implicated, these prior studies did not assess the change in ferroptosis sensitivity of the resistant sublines. The inventors found that upon acquisition of BRAFi/MEKi resistance M249, Me1888 and A375 all upregulate PPARGC1A (PGC1-α) (57), have distinct but overlapping patterns of upregulation of mitochondrial respiration programs (tricarboxylic acid cycle (TCA), electron transport chain (ETC), oxidative phosphorylation, and mitochondrial biogenesis), and all upregulate lipid oxidation pathways (featured by PPARα and ACOX1 (62)). In sum, these changes may cause higher dependence on glutathione metabolism for lipid detoxification via GPX4, while all cases do not equally upregulate their ROS detoxification pathways (FIG. 23E-F).

In accordance with this last observation, i) expression of the ROS detoxification pathway gene glutathione synthetase (GSS) and inferred activity of the ROS detoxification pathway are downregulated in both dedifferentiation and BRAF amplification cases of MAPKi resistance (FIG. 23G-H), and ii) the levels of reduced glutathione (GSH) are decreased upon both dedifferentiation (39) and upon BRAF amplification (FIG. 23I). The inventors furthermore found that the NCOA4 (nuclear receptor coactivator 4) gene, that mediates the selective autophagic degradation of ferritin (63) is upregulated in both dedifferentiation- and BRAF amplification-mediated MAPKi resistant cells, but is downregulated in NRAS mutation-mediated resistance (where both parental and resistant sublines have similar ferroptosis sensitivity (39)) (FIG. 23E). This observation is in line with a previous study finding that NCOA4 promotes accumulation of cellular labile iron, leading to higher susceptibility to pro-ferroptotic drug (64).

Taken together, although dedifferentiation-mediated MAPKi resistance has distinctions from BRAF amplification-mediated resistance, they both demonstrate increased GPX4 inhibition (RSL3) sensitivity, have a common pattern of downregulated ROS detoxification genes such as GSS, and demonstrate upregulation of the iron homeostasis regulator NCOA4.

### C. Discussion

Focal amplifications of oncogenes in either DM- (ecDNA-) or HSR-mode are clinically observed both as a resistance mechanism for inhibitors targeting oncogenes (e.g. MET in EGFRi-treated lung cancer (65)) and in the targeted therapy-naïve setting (e.g. MYCN in neuroblastoma(27,66)). The disappearance of oncogene-containing DMs has also been reported upon modeling of oncogene-targeted therapy (67). While a few-fold amplification of BRAF is sometimes observed in treatment-naïve melanoma tumors (68), higher-fold DM or HSR focal amplifications are typically seen only following MAPK inhibitor therapy (data not shown). To further elucidate the genomic plasticity enabled by focal amplifications, the inventors developed an expanded version of a BRAF+MEK inhibition and BRAF locus amplification model. This system demonstrated a high degree and broad range of evolutionary plasticity of BRAF amplicon in response to changing drug dose regiments, which can be in part generalized to other amplified MAPK genes mediating resistance (i.e., RAF1 and NRAS). BRAF plasticity was in cases coupled to multiple genomic rearrangement and related mechanisms such as kinase domain duplications and alternative splicing.

In the initial phase of drug resistance to dual BRAF and MEK inhibition, the BRAF amplification appeared via DMs. Under conditions of a stable double drug dose, the population gradually became dominated by an HSR-form of BRAF amplification. Such DM to HSR conversion was also observed in single-cell-derived clones of the M249-VSR cells, supporting that de novo integrations of (potentially agglomerated (45)) DMs did occur in addition to selection of an existing HSR+ population. Such FA mode switch is also supported by the conserved genomic contents and shared breakpoints between DM and HSR amplicons based on WGS and OM data. This mode switch result adds to reports in the literature for other focally amplified oncogenes in different cancer types. For example, one study conducted a long-term observation on a non-drug-treated leukemia cell line and saw formation of MYC-carrying HSRs from DMs (25). Another study proposed a common origin of MYCN DM and HSR in neuroblastoma based on their shared structures (69).

The reproducible observation of the DM to HSR transition led us to hypothesize that DMs carry a higher fitness disadvantage than HSRs during stable conditions. In support of this, the inventors found that an oscillating drug dose could prevent or prolong autosomal integration of the amplicon. This difference in fitness is arguably linked to uneven segregation of DMs (13-16), and the resulting uneven BRAF gene copy numbers in daughter cells. During non-stable conditions, cellular heterogeneity provided by uneven segregation can provide a reservoir of cells more adept to grow well in the new conditions. In contrast, during stable drug-dose conditions a reduction in cellular heterogeneity would produce a fitness advantage, which all daughter cells maintaining the optimal BRAF gene copy number. The inventors also observed DM numbers tended to decrease during long term stable culture, probably suggesting that a secondary (undetermined) resistance mechanism allowed these cells to depend less on the DMs (FIG. 11A-B). Taken together, these results along with other findings on the evolution of DMs harboring different oncogenes in different cancer types (25,28,29), support that in some cell types, in non-changing contexts DMs are not a fitness optimized form of amplification, and thus tend to be replaced by other mechanisms such as less heterogeneous chromosomally integrated HSRs. However, such fitness considerations are likely impacted by cell-type and by the characteristics of the oncogene driving the focal amplification.

In contrast, in non-constant conditions, such as the tumor microenvironment or tumors targeted by therapeutics, the uneven segregation of DMs provides an evidence-supported model for tumor heterogeneity that in turn provides tumors the diversity to withstand changes in conditions that impact fitness (13-16,70). In the single and double drug withdrawal experiments involving DM+ cells, the inventors saw rapid decreases in the DM copy number (e.g. BRAF or RAF1). It is possible that the rapid changes in DM copy number were due to selection of a pre-existing DM-negative subpopulation or that post-mitosis cells with less DMs due to uneven segregation could have been selected for upon drug withdrawal (13-16). It is also possible that DMs were exported out of cells through previously observed micronuclei exclusions (71), especially in the single drug withdrawal cases where decreases in DM copy number occurred without appreciable changes in cell viability or growth rates. The single drug withdrawal results also support that dual BRAF and MEK inhibition is required to sustain pressure for high copies of the BRAF gene.

Beyond DM plasticity, this study revealed that ‘HSR plasticity’ can also be a mode of tumor evolution in response to drug challenge. Dose reduction experiments demonstrate that HSRs can offer somewhat comparable levels of plasticity as DMs. Due to the inherent differences between DM and HSR modes of amplification, this is almost undoubtedly through distinct molecular mechanisms. In more detail, the inventors observed single-cell-derived HSR-containing cell populations that demonstrated dose-tunable BRAF and RAF1 HSR lengths. OM, WGS and FISH data reveal that such length shorting involves reducing the number of amplicon repeats rather than changing integration junctions (FIG. 4H-I). Future work will investigate whether errors and repairs made while replicating and segregating intrachromosomal long HSRs may be generating heterogeneity and thus contributing to this plasticity.

In sum, the single cell clone results support that de novo genetic alterations occur during expansion form a single cell, and/or during the stress of drug withdrawal, thus creating population heterogeneity and enabling population plasticity. In these cases, selection alone cannot explain the outcome, and clearly genomic instability, in the HSR case potentially mediated by the challenge of replicating adjacent homogeneous regions, is diversifying the population.

The tumor evolutionary and drug resistance plasticity enabled by focal amplifications extended beyond changes in amplicon copy numbers and DM versus HSR modes. In particular the inventors observed two additional parallel mechanisms, i) kinase domain duplication, representing an additional genomic rearrangement mechanism (35,53,54), and ii) activation of an alternative splicing mechanism (34). These results indicate cells harboring BRAF KDD-encoded DMs mechanism can be reproducibly selected from an HSR-predominant population upon dual MAPKi escalation treatment due to an accompanying gain in relative fitness advantage. Further research on KDD formation and KDD-mediated resistance could offer therapeutic insights for pan-cancer therapy, as this alteration occurs to many other kinases, such as EGFR and FGFR1 in glioma and lung cancer (72-75). The inventors also observed the alternative splicing mechanism as a potential method to escape reliance on high DM copy number during an oscillating dose regiment. The drug resistance provided by the splice variant, arguably lowers the number of DMs required, but maintains the DM-mediated unequal segregation-based heterogeneity.

Therapeutic approaches to target the vulnerabilities of FA-harboring cells are in academic and industry development. This study demonstrates important challenges, such as mode switching and acquisition of additional genomic rearrangements, that must be co-addressed in these pursuits. Here the inventors report that BRAF-amplified melanomas relapsed from dual MAPKi treatment show increased ferroptosis sensitivity, which extends the spectrum of ferroptosis sensitivity in melanoma therapy resistance. The inventors found that an additional mechanism, distinct from treatment-induced dedifferentiation and mesenchymal transition, can generate sensitivity to GPX4 inhibition (39). This finding links to studies of MAPKi-induced oxidative stress in melanoma. In some melanomas, BRAF^{V600E} activation leads to enhanced glycolysis and reduced oxidative phosphorylation and mitochondrial respiration (57). However, BRAF inhibition, including acquired resistance to BRAF inhibitors, can switch the energy generation dependency back to oxidative phosphorylation pathway by induction of PPARGC1A and overexpression of other mitochondrial genes (57-60,76,77). Reactive oxygen species (ROS) productively mediate redox-based energy production in mitochondrial respiration, but they can also damage lipid, protein and DNA (78). Hence respiring cells need to upregulate detoxification programs to compensate for elevated oxidative stress (61,79). The imbalance of cellular prooxidative and antioxidative mechanism can lead to cell death (80). Ferroptosis is one form of cell death that can result from such compromised redox homeostasis, mediated by iron-dependent accumulation of lipid peroxides (81).

In these studies, BRAF amplification-mediated MAPKi resistant melanoma cells did not exhibit dedifferentiation. However, they did downregulate GSS and had limited reduced glutathione levels, which would limit their capacity to detoxify lipid ROS (FIG. 23E-I). They also upregulated the iron homeostasis regulator NCOA4 (64,82) (FIG. 23E), similar to other MAPKi parental/resistant melanoma pairs with differential RSL3 sensitivity, consistent with higher vulnerability to ferroptosis induction. Relatedly, one previous report found that MAPKi acquired resistance through most MAPK reactivation mechanisms, such as RTK overexpression, NRAS^{Q61H/Q61K} mutation, KRAS^{G12C} mutation, BRAF splice variant and BRAF amplification, are all more vulnerable to undergo apoptosis via inhibition of the system x_{c}⁻ cystine/glutamate antiporter in cells treated with an HDAC inhibitor (83). These finding complements this by uncovering a different form of cell death, ferroptosis, occurring under a similar MAPKi resistance context, and extends the role of ferroptosis in MAPKi resistance beyond cases of dedifferentiation. Taken together, the melanoma dedifferentiation-independent synthetic lethality between BRAF amplification and ferroptosis identified here provides therapeutic insight for treating BRAF amplified melanomas relapsed from MAPKi treatment. While ferroptosis sensitivity was observed in both the HSR and DM harboring cells, previous reports have revealed that DM and HSR can have specific targetable vulnerabilities linked to their distinct mechanisms for generation and maintenance (84-90). Future work is needed to confirm such FA-mode-specific vulnerabilities in BRAF amplification systems.

Collectively, the inventors observed a high degree and broad range of tumor evolution and drug resistance plasticity enabled by or coupled to focal amplifications. Through perturbations by a panel of drug regiment challenges, the inventors observed i) de novo generation of extrachromosomal DMs, ii) de novo integration of DMs into chromosomal HSRs, iii) context-dependent HSR-mediated fitness advantage over DMs, iv) context-dependent DM-mediated fitness advantage over HSRs, v) co-evolution of DMs and a de novo genomic rearrangement creating a kinase domain duplication, vi) co-evolution of DMs and activation of BRAF alternative splicing, vii) propensity to couple secondary resistance mechanisms (KDD and/or alternative splicing) to DMs to reduce the total number of DMs required, and viii) a plasticity of HSRs that compares in some kinetic aspects to the known plasticity of DMs. Appreciation of the interplay of focal amplification modes with drug regiments and other resistance mechanisms is central to one's understanding of tumor evolution and drug resistance, and to developing therapeutic approaches to overcome the resulting plasticity.

### D. Methods

### 1. Cell Culture Conditions, Xenografts and Generation of Drug-Resistant Cell Lines

The M249 (RRID:), M395 (RRID: CVCL_XJ99) and M245 NRAS^{Q61K} (RRID: CVCL_D754) cell lines are part of the M series melanoma lines established from patient biopsies at UCLA under UCLA IRB approval #02-08-06 and were obtained from Dr. Antoni Ribas (91). PDX1 (NRAS^{Q61R}) and PDX13 (BRAF^{S365L}) cell lines were derived from patient-derived xenografts with the same names (47). M245 C3 and C5 sublines were reported previously (52). Me1888 (RRID: CVCL_4632) and A375 (RRID: CVCL_0132) cells lines and their variants were described previously (35,43). All cell lines have been tested for mycoplasma. All cells were cultured in RPMI 1640 with L-glutamine (Gibco), 10% (v/v) fetal bovine serum (Omega Scientific), and 1% (v/v) streptomycin (Gibco). All cells were maintained in a humidified 5% CO2 incubator. Resistance M249 cell lines were generated by exposing cells to step-wise increasing doses of vemurafenib and selumetinib, similar to the previously described approach (41). Briefly, the doses for both drugs were sequentially increased by roughly 2-fold, with each dose escalation taking place when cells resumed growth rates with doubling in 4 days or less. The initial and final doses were 0.05 μM and 2 μM, respectively. Growth and viability were assayed by staining cells with trypan blue (Sigma-Aldrich) followed by cell counting using Vi-cell XR Cell Viability Analyzer (Beckman Coulter) or by CellTiter-Glo luminescence assay. Doubling times for M249 SCs and bulk cells were calculated by fitting exponential growth curves, and their error bars were derived based on a previously published method (92). Cells were only sampled for experiments when they show reasonable growth rate at corresponding dose.

### 2. Inhibitors

BRAF inhibitors vemurafenib and dabrafenib as well as and MEK inhibitors selumetinib and trametinib were obtained from Selleckchem or LC Laboratories. Pro-ferroptotic drugs RSL3 and Erastin were obtained from Cayman Chemical and Selleckchem, respectively. Ferroptocide was described previously (56). DNA-PK inhibitor NU7026 was purchased from Selleckchem. Inhibitors were all dissolved in DMSO.

### 3. Single-Cell-Derived Clones

Resistant subclones were derived by seeding single cells from the bulk population into 96-well plates using FACSAria cell sorter. Doublets are removed by circling the right area in the FSC-height vs area plot. Seeded single cells were then cultured using aforementioned medium or a modified medium with 20% FBS for two weeks. Culture medium was not changed until clear colonies were observed in some wells. If certain treatments are needed, i.e. double drug dose changes, they are initiated upon seeding the cells. M245 resistance subclones were derived by ring selection (47).

### 4. Cytogenetics

Cells were blocked at metaphases by adding colcemid (KaryoMax, Thermo Fisher Scientific) at a final concentration of 0.05 μg/ml followed by incubation at 37° C. for 6-8 hours. Cells were then fixed using methanol:acetic acid (3:1). FISH slides were prepared by dropping fixed cells in a humid environment following the manufacture's protocol provided by Cytotest and Empire Genomics. FFPE xenograft tumor FISH slides were prepared by pepsin digestion followed by similar procedures of cell line FISH. Colored FISH images were taken and processed using confocal microscope Leica TCS SP8 X. Karyotype categorizations were based on the guidelines in FIG. 8. The fractions under certain images represent the number of cases for corresponding karyotype divided by total number of cases analyzed. If not otherwise mentioned, scale bars in FISH images represent 10 μm. Centromere probe names are abbreviated as CEN-x. DM numbers were quantified by directly counting the number of features in the FISH images or by using ecDNA quantification tool EcSeg (93). HSR lengths were quantified by dividing the probe area by chromosomal DAPI area in metaphases. The staining areas were calculated using ImageJ v1.53a. Cells fixed by the same procedure were also used for G-banding. G-banded metaphase spreads were photographed using 80i Nikon Microscope and Applied Spectral Imaging (ASI) Karyotyping system. A minimum of ten metaphases were karyotyped.

5. qPCR-Based BRAF Copy Number Assay

qPCRs for BRAF genomic DNA (gDNA) copy number measurement were performed by combining samples with PowerUp SYBR Green Master Mix (Applied Biosystems) in Optical 96-Well Reaction Plates (Applied Biosystems) with three technical replicates for each sample. Plates were then read by 7500 Real-Time PCR System (Applied Biosystems) using the standard cycling mode. Input templates for all samples were genomic DNAs extracted using DNeasy Blood & Tissue Kits (Qiagen). Unless specified, all qPCR runs used M249 parental as the reference sample and GAPDH as the endogenous control. Error bars represent t-distribution-based 95% confidence intervals from triplicates: RQ_{max/min}=2^{-ΔΔCt±t}^{_{0.05,df*}}^{SE}. RQ: relative quantity. Ct: threshold cycle. df: degree of freedom. SE: sample standard error. All primers were ordered from Eurofins Scientific and their sequences are shown below.

| | BRAF Forward: |
|---|---|
| (SEQ ID NO: 1) | |
| | 5′-TTTAGAACCTCACGCACCCC-3′ (intron 2) |
| | BRAF Reverse: |
| (SEQ ID NO: 2) | |
| | 5′-TGTTGTAGTTGTGAGCCGCA-3′ (intron 2) |
| | GAPDH Forward: |
| (SEQ ID NO: 3) | |
| | 5′-CTGGCATTGCCCTCAACG-3′ |
| | GAPDH Reverse: |
| (SEQ ID NO: 4) | |
| | 5′-AGAAGATGAAAAGAGTTGTCAGGGC-3′ |

### 6. Comparative Genomic Hybridization and Low-Pass Whole Genome Sequencing

Genomic DNA of M249-P and M249-VSR cells were isolated by using DNeasy Blood & Tissue Kits (Qiagen). Samples were run on Agilent 6×80K array. The raw data was then processed by Cytogenomics software v5.2 (Agilent Technologies). Nested genomic regions were flattened and .seg files were generated, followed by data visualization in IGV v2.10.0 (94). Regions with large copy number changes were identified by comparing every segment in M249-VSR with the corresponding segment in M249-P. The same genomic DNAs were sent to PacGenomics for low-pass WGS with coverage of 0.04. Library was prepared using KPA DNA Library Preparation Kit. Sequencing was performed on Illumina NextSeq 500 using 75 bp paired end reads (2×75 bp). CNA was inferred using Ginkgo v3.0.0 (95), which contains a step that used bowtie v1.2.1 (96) to align raw reads to hg19 genome.

### 7. Whole Genome Sequencing, Copy Number and Structural Variant Calling of M249 Series

Genomic DNA of M249-P and M249-VSR sublines were extracted by DNeasy Blood & Tissue Kits. The samples underwent whole-genome sequencing library preparation and then sequenced on Illumnia Novaseq S1 at 2×150 and 10-15× coverage. Raw reads in fastq files were aligned to hg38 using BWA-MEM v0.7.1 (97). The duplicated reads were marked by MarkDuplicates tool from GATK v4.1.2 (98). Next, CNA calls were performed using CNVkit v0.9.7 (99) with flat normal as the control. Segmentation was performed using hmm-tumor method. CNVkit results were used the input for AmpliconArchitect vi.2 (100). The same genomic region chr7:139410000-141180000, which corresponds to BRAF amplicon, was used as the seed interval for all M249 samples when running AmpliconArchitect. Structural variants were also called using SvABA v1.1.3 (101) for analyzing break points and integration junctions.

### 8. AmpliconReconstructor Analysis

AmpliconArchitect-generated breakpoint graphs were first converted to in silico digested optical map segments. AmpliconReconstructor v1.01 (45) (https://github.com/jluebeck/AmpliconReconstructor) was then run with default settings on the breakpoint graph segments and the assembled Bionano contigs from the Bionano Genomics optical genome map de novo assembly pipeline. From the collection of reconstructed breakpoint graph paths present, we identified circular or non-circular paths representing the ecDNA or HSR structures. Resulting structures were visualized with CycleViz v0.1.1

### 9. Generation of Optical Mapping Data

Ultra-high molecular weight (UHMW) DNA was extracted from frozen cells preserved in DMSO following the manufacturer's protocols (Bionano Genomics, USA). Cells were digested with Proteinase K and RNAse A. DNA was precipitated with isopropanol and bound with nanobind magnetic disks. Bound UHMW DNA was resuspended in the elution buffer and quantified with Qubit dsDNA assay kits (ThermoFisher Scientific). DNA labeling was performed following manufacturer's protocols (Bionano Genomics, USA). Standard Direct Labeling Enzyme 1 (DLE-1) reactions were carried out using 750 ng of purified UHMW DNA. The fluorescently labeled DNA molecules were imaged sequentially across nanochannels on a Saphyr instrument.

De novo assemblies of the samples were performed with Bionano's de novo assembly pipeline (Bionano Solve v3.6) using standard haplotype aware arguments. With the Overlap-Layout-Consensus paradigm, pairwise comparison of filtered DNA molecules (average length approximately 350 kbp) of >200× coverage was used to create a layout overlap graph, which was then used to generate the initial consensus genome maps. By realigning molecules to the genome maps (P value cut off <10⁻¹²) and by using only the best matched molecules, a refinement step was done to refine the label positions on the genome maps and to remove chimeric joins. Next, during an extension step, the software aligned molecules to genome maps (P<10⁻¹²), and extended the maps based on the molecules aligning past the map ends. Overlapping genome maps were then merged (P<10⁻¹⁶). These extension and merge steps were repeated five times before a final refinement (P<10⁻¹²) was applied to “finish” all genome maps.

### 10. FaNDOM Analysis

Optical map alignment of Bionano contigs to the reference genome and Bionano raw molecules to the reference genome was performed with FaNDOM v0.2 (102). Alignment and SV detection was done by calling modules named ‘wrapper_contigs.py’ and ‘wrapper_individual.py’ with default settings. Alignments were visualized with the MapOptics v1.0.0 (103) software.

### 11. RNA-Seq Analysis

Total RNA was isolated from M249-P and M249-VSR cells by using RNeasy Plus Mini Kit (Qiagen). Samples were sequenced on HiSeq3000 at 150 bp paired-end or on Novaseq SP at 50 bp paired-end. Raw data was then processed using Toil v3.9.1 pipeline to output transcripts per million (TPM), including STAR v2.7.1a that aligned raw reads to GRCh38 genome (104,105). Following data trimming and log transformation, visualizations were done in R. For calculating allele frequencies of BRAF^{V600E}, all RNA-seq fastq files were aligned using STAR, and the resultant bam files were processed according to GATK RNA-seq short variant discovery best practices until the step of haplotype calling (106). For visualization, we loaded base quality score recalibrated bam files to IGV.

### 12. Immunoblotting and Antibodies

Cell lysates were prepared by using mRIPA buffer supplemented with PMSF, leupeptin and aprotinin. Western blots were performed using following antibodies: beta-actin (AC-15, Sigma-Aldrich), beta-actin (13E5, Cell Signaling Technology), BRAF (F-7, Santa Cruz Biotechnology), BRAF (C-19, Santa Cruz Biotechnology), Goat anti-Rabbit secondary antibodies (IRDye 680RD, LI-COR), Goat anti-Mouse secondary antibodies (IRDye 800CW, LI-COR). Images were directly output by Odyssey CLx Imaging System (LI-COR).

13. Reverse Transcriptase (RT)-PCR and -qPCR

Total RNA was extracted from fresh cells using RNeasy Plus Mini Kit (Qiagen). Reverse transcriptions were then performed by using SuperScript VILO cDNA Synthesis Kit (Invitrogen). cDNA was then used for PCR and qPCR. Primers for detecting exon18-10 and exon9-10 junctions were the same as what previously published (35). The regular PCR was performed using Phusion High-Fidelity PCR Master Mix with HF Buffer (New England Biolabs). The PCR products that targeted exon18-10 and exon9-10 were then combined for each sample and run on 2% agarose gel. For qPCR, each sample was combined separately with PowerUp SYBR Green Master Mix (Applied Biosystems) and loaded on Optical 96-Well Reaction Plates (Applied Biosystems) in triplicate. Plates were then read by 7500 Real-Time PCR System (Applied Biosystems) using the standard cycling mode.

### 14. Replica Plating Screen for DM-KDD Subpopulation

Each of 41 Single cells derived clones (SCs) of M249-VSR-HSR cells (cultured at 2 μM VEM+SEL) was seeded in 6 wells of 96-well plates with the same cell number per well. Three wells of each clone were treated by 5 μM VEM+SEL white the other three stayed at 2 μM. After 6 days, cell viabilities were measured by CellTiter-Glo Luminescent Cell Viability Assay. 13 of 41 SCs were picked for a second round of the dose increase screen to confirm the findings. The viability of SCs was visualized by heatmaps using the R package ComplexHeatmap v2.6.2 (107).

### 15. Barcode-Based Clone Tracing

ClonTracer Barcoding Library (55) was purchased from Addgene. The plasmid pool was expanded by electroporation transformation. Lentivirus was made by transfecting 293T cells. M249-VSR-HSR cells were tested for their puromycin dose-response and multiplicity of infection curves. For the actual infection, 54 million M249 HSR cells were spin-infected in 12 well plate with 8 μg/ml polybrene, followed by a six-day puromycin (0.3 μg/ml) selection. Day 0 refers to the end of the selection. Next, cells underwent a standard culture growth period with kinase inhibitors present until the genomic DNA collection points on day 14 and day 35. The sequencing library was prepared by PCR amplification of barcode regions using the primer sequence provided by the manufacturer. The libraries were paired-end sequenced on Illumina NextSeq 500 at 75 bp read length.

### 16. Analysis of Copy Number Data for MAPKi Treated Melanoma

MAPKi treated melanoma copy number profiles from multiple previous studies were downloaded and compiled. The software packages used for CNA callings include CNVkit v0.9.7 (99), penncnv v1.0.5(115), CopywriteR v1.3(116), rCGH v1.20.0(117) and the circular binary segmentation (CBS) algorithm (118). When the actual normal samples are not available for certain patients, flat normals were used to call CNA. Gene level copy numbers of BRAF were determined by averaging all length normalized segments in BRAF genomic region after removing the gaps.

### 17. Simulation of BRAF Amplification Boundaries

For FIG. 6C, the expectation of amplicon boundaries around BRAF was simulated using a method from previous study (11). Briefly, to construct the solid line, real copy number profiles of treated melanoma samples used include those that have both pre-treatment and post-progression time points with BRAF CN log 2(post/pre)>0.75 and BRAF CN log 2(post/normal) >1.3 as well as those don't have pre-treatment data available and BRAF CN log 2(post/normal)>1.7. We have confirmed all selected samples have focal BRAF amplification instead of arm level. For the dashed line, random amplicons were generated by shifting each of real BRAF amplicon boundaries multiple times but still encompass BRAF gene. The boundaries are sometimes defined after merging nearby CNA segments with log 2 differences within 1 and gaps smaller than 1 Mb. The genome was binned at 10 kb size. For each bin, amplification frequency is defined by the percentage of samples that have BRAF CN log 2(post/normal)>1.3.

### 18. Dose Response Curve

The dose response curves of ferroptosis inducing agents RSL3, Erastin and Ferroptocide were performed by seeding appropriate number of cells on day 0 in 96-well plates, treating cells on day 1 with corresponding drugs and reading the plates on day 4 for viability using CellTiter-Glo luminescent assay. If not otherwise mentioned, resistance cells were maintained in full dose of MAPK inhibitors throughout the dose response experiments to keep the BRAF amplifications. Seeding density for each cell line was determined by using the same assay and the same experimental length with multiple cell number titrations. The dose series were generated by serial dilutions. All drugs used for dose response curves were dissolved in DMSO. DMSO toxicity was performed on the cell lines to determine the appropriate DMSO concentration (0.5%), which was used in all doses. The resulting values from viability assays were normalized to the zero-dose condition after subtracting background (wells with no cells). The curve fittings were performed by using three-parameter model in dre v3.0-1 R package (119).

19. Viability Assay for Inducing and Protecting from Ferroptosis

6000 cells were seeded per well in 96-well plates and treated with ferroptosis inducing agents in combination with vehicle, GSH (Sigma, G4376), Trolox (Acros Organics, 218940010) or DFO (Sigma, D9533) next day. CellTiter-Glo luminescence was assessed 24 hr after treatment. For quantification, all values are normalized to vehicle conditions. Resistance cells were maintained in full dose of MAPK inhibitors throughout the treatments to keep the BRAF amplifications.

### 20. ROS Measurements

In 12-well plates, 80000 M249-P and M249-VSR-DM cells were seeded per well and treated with next day with RSL3 in combination with Trolox or vehicle. Resistance cells were maintained in full dose of MAPK inhibitors keep the BRAF amplifications. After 24 hr, CM-H2DCFDA dye (Invitrogen C6827) was added to each well and incubated for another 20 min at 37° C. Cells were then washed with PBS, harvested by trypsinization, suspended in 250 ml PBS and filtered through cell strainers. The samples were analyzed with BD LSRII Analytic Flow Cytometer at the excitation wavelength of 488-nm.

### 21. Metabolomics-Based Glutathione Measurement

Appropriate number of cells were seeded 10 cm dishes for 72 hr growth to reach 80% confluency. MAPKi-resistant cells were maintained in full dose of VEM+SEL keep the BRAF amplifications. On the day of collection, cells were rinsed with ice-cold 150 mM NH4AcO at pH 7.3, incubated with 80% MeOH at −80° C. for 20 minutes, scrapped off from the plates and transferred to Eppendorf tubes. Cells are then vortexed for 10 seconds and centrifuged at 16000 g for 15 minutes at 4° C. The supernatants were transferred to glass vial and dried in Genevac EZ-2 Elite evaporator at 30° C. to obtain metabolite extracts.

Dried metabolites were resuspended in 50% acetonitrile (ACN):water and 1/10th was loaded onto a Luna 3 um NH2 100A (150×2.0 mm) column (Phenomenex). The chromatographic separation was performed on a Vanquish Flex (Thermo Scientific) with mobile phases A (5 mM NH4AcO pH 9.9) and B (ACN) and a flow rate of 200 l/min. A linear gradient from 15% A to 95% A over 18 min was followed by 9 min isocratic flow at 95% A and re-equilibration to 15% A. Metabolites were detection with a Thermo Scientific Q Exactive mass spectrometer run with polarity switching (+3.5 kV/−3.5 kV) in full scan mode with an m/z range of 70-975 and 70.000 resolution. TraceFinder 4.1 (Thermo Scientific) was used to quantify the targeted metabolites by area under the curve using expected retention time and accurate mass measurements (<5 ppm). Values were normalized to protein content of extracted material. Data analysis was performed using in-house R scripts.

Data analysis of melanoma dedifferentiation, ferroptosis and ROS related program

Raw RNAseq data of Me1888, Mel888-DTR, A375, A375-DTR, SKMEL28P, SKMEL28R and M series cell lines were downloaded from corresponding GEO accessions (39,43,120-124). The data was processed through Toil v3.9.1 (105) to obtain RSEM (125) expected counts and normalized by log-transformed counts per million (logCPM) approach. Principle component analysis (PCA) was performed using mean-centered logCPM values of M series cell lines and serve as the framework, on which RNAseq data of other samples were projected onto for determining their dedifferentiation stages. The scores of selected gene sets for the parental/resistance-paired cell lines was calculated using the single sample gene set enrichment analysis (ssGSEA) method in GSVA v1.38.2 R package (126). Nearly all gene sets were taken from MSigDB v7.4 (127,128) except for ROS detoxifying gene sets which was made by combining i) a subset of detoxifying genes (a combination of multiple detoxifying gene sets in MSigDB) that correlate well (Pearson correlation >0.4) with the dedifferentiation trajectory scores of M series samples and ii) top 8 genes that downregulate upon knocking down PGC1α in A375 cells (61).

### 22. Statistical Analysis and Visualization

Most statistical analysis and data visualizations were performed using R v4.0.3 in RStudio v1.3.1093.

### E. Tables

Supplementary Table S1. Optical mapping-inferred amplicon junctions for M249 VSR.

| | | | | | | Number of Supporting Molecules | | | | | Depth Normalized Number of Supporting Molecules | | | | |
|---|---|---|---|---|---|---|---|---|---|---|---|---|---|---|---|
| | | | | Break- | | | SC2-2- | | | | | SC2-2- | | | |
| | | | | point | | SC2 | 0.1 | | | | SC2 | 0.1 | M249- | | |
| Chr | | Chr | | Karyo- | junction | Long | Short | M249-VSR-DM | SC401 | M249-P | Long | Short | VSR-DM | SC401 | M249-P |
| 1* | Pos1† | 2* | Pos2† | type | type | HSR | HSR | DM | DM | no FA | HSR | HSR | DM | DM | no FA |
| 7 | 139,518K | 7 | 141,069K | S | head-to- | 850 | 16 | 403 | 250 | 0 | 0.00620012 | 0.000117899 | 0.002154309 | 0.002422035 | 0 |
| | | | | | tail | | | | | | | | | | |
| 3 | 196,788K | 7 | 140,520K | B | chromosome | 14 | 12 | 0 | 0 | 0 | 0.00010212 | 8.84241E−05 | 0 | 0 | 0 |
| | | | | | integration | | | | | | | | | | |
| 3 | 196,830K | 7 | 140,520K | A | chromosome | 10 | 8 | 0 | 0 | 0 | 7.29426E−05 | 5.89494E−05 | 0 | 0 | 0 |
| | | | | | integration | | | | | | | | | | |
| 7 | 139,617K | 7 | 139,907K | C1 | tail-to- | 37 | 1 | 2 | 2 | 0 | 0.000269888 | 7.36867E−06 | 1.06914E−05 | 1.93763E−05 | 0 |
| | | | | | tail | | | | | | | | | | |
| 7 | 139,632K | 7 | 140,281K | C3 | tail-to- | 37 | 0 | 1 | 1 | 0 | 0.000269888 | 0 | 5.34568E−06 | 9.68814E−06 | 0 |
| | | | | | tail | | | | | | | | | | |
| 7 | 139,822K | 7 | 140,188K | C2 | head-to- | 19 | 0 | 0 | 0 | 0 | 0.000138591 | 0 | 0 | 0 | 0 |
| | | | | | tail | | | | | | | | | | |
| 7 | 140,568K | 7 | 140,724K | I | head-to- | 96 | 2 | 5 | 5 | 1 | 0.000700249 | 1.47373E−05 | 2.67284E−05 | 4.84407E−05 | 6.17592E−06 |
| | | | | | head | | | | | | | | | | |
| | | | | | inversion | | | | | | | | | | |
| 7 | 139,933K | 7 | 139,955K | C4 | tail-to- | 0 | 7 | 0 | 0 | 0 | 0 | 5.15807E−05 | 0 | 0 | 0 |
| | | | | | tail | | | | | | | | | | |
| 7 | 139,863K | 7 | 141,059K | C5 | head-to- | 0 | 13 | 0 | 0 | 0 | 0 | 9.57927E−05 | 0 | 0 | 0 |
| | | | | | tail | | | | | | | | | | |

| | | | | | |
|---|---|---|---|---|---|
| Total Number of Molecules | 13709413 | 13570966 | 18706692 | 10321900 | 16191909 |

SUPPLEMENTARY TABLE S2
WGS-inferred amplicon junctions for M249 VSR.

| | | | | number of | | corrected |
|---|---|---|---|---|---|---|
| | | | svaba insert | supporting | | insert |
| sample | ID | svaba junction* | sequence* | reads | corrected junction† | sequence† |
| M249- | M249-VSR- | chr7:141,085,190(+) | C | 223 | chr7:141,085,190(+) to | C |
| VSR- | DM_S2 | to chr7:141,105,472(−) | | | chr7:141,105,472(−) | |
| DM | | | | | | |
| M249- | M249-VSR- | chr7:141,125,773(+) | ATCACTTACACA | 103 | chr7:141,125,773(+) to | ATCACTTAC |
| VSR- | DM_S2 | to chr7:141,129,896(−) | TCAGAAAGCTGG | | chr7:141,129,896(−) | ACATCAGAA |
| DM | | | TTATA (SEQ | | | AGCTGGTTA |
| | | | ID NO: 25) | | | TA (SEQ |
| | | | | | | ID NO: 25) |
| M249- | M249-VSR- | chr7:141,129,975(+) | CATTTTA | 161 | chr7:141,129,975(+) to | CATTTTA |
| VSR- | DM_S2 | to | | | chr7:141,135,422(+) | |
| DM | | chr7:141,135,422(+) | | | | |
| M249- | M249-VSR- | chr7:141,085,174(+) | AGTAGAGATGGG | 232 | [[chr7:141,085,190(+) to | [[C]] |
| VSR- | HSR_S3 | to chr7:141,105,476(−) | GTC (SEQ ID | | chr7:141,105,472(−)]] | |
| HSR | | | NO: 26) | | | |
| M249- | M249-VSR- | chr7:141,125,773(+) | ATCACTTACACA | 61 | chr7:141,125,773(+) to | ATCACTTACA |
| VSR- | HSR S3 | to chr7:141,129,896(−) | TCAGAAAGCTGG | | chr7:141,129,896(−) | CATCAGAAAG |
| HSR | | | TTATA (SEQ | | | CTGGTTATA |
| | | | ID NO: 25) | | | (SEQ ID NO: |
| | | | | | | 25) |
| M249- | M249-VSR- | chr7:141,129,975(+) | CATTTTA | 144 | chr7:141,129,975(+) to | |
| VSR- | HSR_S3 | to chr7:141,135,422(+) | | | chr7:141,135,422(+) | |
| HSR | | | | | | CATTTTA |
| SC1 | M249-VSR- | chr7:141,085,175(+) | AGTAGAGATGGG | 233 | [chr7:141,085,190(+) to | [[C]] |
| | 181005- | to chr7:141,105,476(−) | GTC (SEQ ID | | chr7:141,105,472(−)]] | |
| | SC1_S7 | | NO: 26) | | | |
| SC1 | M249-VSR- | chr7:141,125,773(+) | ATCACTTACACA | 100 | chr7:141,125,773(+) to | ATCACTTACA |
| | 181005- | to chr7:141,129,896(−) | TCAGAAAGCTGG | | chr7:141,129,896(−) | CATCAGAAAG |
| | SC1_S7 | | TTATA (SEQ | | | CTGGTTATA |
| | | | ID NO: 25) | | | (SEQ ID NO: |
| | | | | | | 25) |
| SC1 | M249-VSR- | chr7:141,129,975(+) | CATTTTA | 141 | chr7:141,129,975(+) to | CATTTTA |
| | 181005- | to | | | chr7:141,135,422(+) | |
| | SC1_S7 | chr7:141,135,422(+) | | | | |
| SC2 | M249-VSR- | chr7:141,085,174(+) | AGTAGAGATGGG | 245 | [[chr7:141,085,190(+) to | [[C]] |
| | 181005- | to chr7:141,105,472(−) | GTC (SEQ ID | | chr7:141,105,472(−)]] | |
| | SC2_S8 | | NO: 26) | | | |
| SC2 | M249-VSR- | chr7:141,125,773(+) | ATCACTTACACA | 227 | chr7:141,125,773(+) to | ATCACTTACA |
| | 181005- | to chr7:141,129,896(−) | TCAGAAAGCTGG | | chr7:141,129,896(−) | CATCAGAAAG |
| | SC2_S8 | | TTATA (SEQ | | | CTGGTTATA |
| | | | ID NO: 25) | | | (SEQ ID NO: |
| | | | | | | 25) |
| SC2 | M249-VSR- | chr7:141,129,975(+) | CATTTTA | 285 | chr7:141,129,975(+) to | CATTTTA |
| | 181005- | to chr7:141,135,422(+) | | | chr7:141,135,422(+) | |
| | SC2_S8 | | | | | |

*svaba junction and insert sequence: the junction and insert sequence shown by svaba local alignment output.
†Corrected junction and insert sequence: the junction and insert sequence after the manual correction as svaba sometimes does not automatically recognize certain sequences as inserts.
[[double brackets]]: there is a correction,
black text: there is no correction.

### F. References

The following references and the publications referred to throughout the specification, to the extent that they provide exemplary procedural or other details supplementary to those set forth herein, are specifically incorporated herein by reference.
1. Hanahan D, Weinberg R A. Hallmarks of cancer: The next generation. Cell. 2011; 144:646-74.
2. Collins S, Groudine M. Amplification of endogenous myc-related DNA sequences in a human myeloid leukaemia cell line. Nature. 1982; 298:679-81.
3. Beroukhim R, Mermel C H, Porter D, Wei G, Raychaudhuri S, Donovan J, et al. The landscape of somatic copy-number alteration across human cancers. Nature. 2010; 463:899-905.
4. Ercan D, Zejnullahu K, Yonesaka K, Xiao Y, Capelletti M, Rogers A, et al. Amplification of EGFR T790M causes resistance to an irreversible EGFR inhibitor. Oncogene. 2010; 29:2346-56.
5. Shi H, Moriceau G, Kong X, Lee M K, Lee H, Koya R C, et al. Melanoma whole-exome sequencing identifies V600E B-RAF amplification-mediated acquired B-RAF inhibitor resistance. Nat Commun. 2012; 3:724-8.
6. Wu S, Turner K M, Nguyen N, Raviram R, Erb M, Santini J, et al. Circular ecDNA promotes accessible chromatin and high oncogene expression. Nature. 2019; 575:699-703.
7. Cox D, Yuncken C, Spriggs A I. Minute Chromatin Bodies in Malignant Tumours of Childhood. Lancet. 1965; 286:55-8.
8. Hamkalo B A, Farnham P J, Johnston R, Schimke R T. Ultrastructural features of minute chromosomes in a methotrexate-resistant mouse 3T3 cell line. Proc Natl Acad Sci USA. 1985; 82:1126-30.
9. Sarokin L, Carlson M. Nucleic Acids Research Nucleic Acids Research. Methods. 1984; 12:8235-51.
10. Zhu Y, Gujar A D, Wong C H, Tjong H, Ngan C Y, Gong L, et al. Oncogenic extrachromosomal DNA functions as mobile enhancers to globally amplify chromosomal transcription. Cancer Cell. 2021; 39:694-707.e7.
11. Helmsauer K, Valieva M E, Ali S, Chamorro Gonzilez R, Schapflin R, Roefzaad C, et al. Enhancer hijacking determines extrachromosomal circular MYCN amplicon architecture in neuroblastoma. Nat Commun. 2020; 11:5823.
12. Morton A R, Dogan-Artun N, Faber Z J, MacLeod G, Bartels C F, Piazza M S, et al. Functional Enhancers Shape Extrachromosomal Oncogene Amplifications. Cell. 2019; 179:1330-1341.e13.
13. LEVAN A, LEVAN G. Have double minutes functioning centromeres? Hereditas. 1978; 88:81-92.
14. Lundberg G, Rosengren A H, Hakanson U, Stewénius H, Jin Y, Stewénius Y, et al. Binomial mitotic segregation of MYCN-carrying double minutes in neuroblastoma illustrates the role of randomness in oncogene Amplification. PLoS One. 2008; 3:e3099.
15. Turner K M, Deshpande V, Beyter D, Koga T, Rusert J, Lee C, et al. Extrachromosomal oncogene amplification drives tumour evolution and genetic heterogeneity. Nature. 2017; 543:122-5.
16. Verhaak R G W, Bafna V, Mischel P S. Extrachromosomal oncogene amplification in tumour pathogenesis and evolution. Nat Rev Cancer. Springer U S; 2019; 19:283-8.
17. Biedler J L, Spengler B A. A novel chromosome abnormality in human neuroblastoma and antifolate-resistant chinese hamster cell lines in culture. J Natl Cancer Inst. 1976; 57:683-95.
18. Toledo F, Le Roscouet D, Buttin G, Debatisse M. Co-amplified markers alternate in megabase long chromosomal inverted repeats and cluster independently in interphase nuclei at early steps of mammalian gene amplification. EMBO J. 1992; 11:2665-73.
19. Ma C, Martin S, Trask B, Hamlin J L. Sister chromatid fusion initiates amplification of the dihydrofolate reductase gene in Chinese hamster cells. Genes Dev. 1993; 7:605-20.
20. Shimizu N, Shingaki K, Kaneko-Sasaguri Y, Hashizume T, Kanda T. When, where and how the bridge breaks: Anaphase bridge breakage plays a crucial role in gene amplification and HSR generation. Exp Cell Res. 2005; 302:233-43.
21. Kaufman R J, Brown P C, Schimke R T. Amplified dihydrofolate reductase genes in unstably methotrexate-resistant cells are associated with double minute chromosomes. Proc Natl Acad Sci USA. 1979; 76:5669-73.
22. Shoshani O, Brunner S F, Yaeger R, Ly P, Nechemia-Arbely Y, Kim D H, et al. Chromothripsis drives the evolution of gene amplification in cancer. Nature. 2021; 591:137-41.
23. Rausch T, Jones D T W, Zapatka M, Stiitz A M, Zichner T, Weischenfeldt J, et al. Genome sequencing of pediatric medulloblastoma links catastrophic DNA rearrangements with TP53 mutations. Cell. 2012; 148:59-71.
24. Carroll S M, DeRose M L, Gaudray P, Moore C M, Needham-Vandevanter D R, Von Hoff D D, et al. Double minute chromosomes can be produced from precursors derived from a chromosomal deletion. Mol Cell Biol. 1988; 8:1525-33.
25. Von Hoff D D, Forseth B, Clare C N, Hansen K L, VanDevanter D. Double minutes arise from circular extrachromosomal DNA intermediates which integrate into chromosomal sites in human HL-60 leukemia cells. J Clin Invest. 1990; 85:1887-95.
26. Coquelle A, Rozier L, Dutrillaux B, Debatisse M. Induction of multiple double-strand breaks within an hsr by meganuclease I-SceI expression or fragile site activation leads to formation of double minutes and other chromosomal rearrangements. Oncogene. 2002; 21:7671-9.
27. Koche R P, Rodriguez-Fos E, Helmsauer K, Burkert M, MacArthur I C, Maag J, et al. Extrachromosomal circular DNA drives oncogenic genome remodeling in neuroblastoma. Nat Genet. 2020; 52:29-34.
28. Ruiz J C, Wahl G M. Chromosomal destabilization during gene amplification. Mol Cell Biol. 1990; 10:3056-66.
29. Kaufman R J, Brown P C, Schimke R T. Loss and stabilization of amplified dihydrofolate reductase genes in mouse sarcoma S-180 cell lines. Mol Cell Biol. 1981; 1:1084-93.
30. Kim H, Nguyen N P, Turner K, Wu S, Gujar A D, Luebeck J, et al. Extrachromosomal DNA is associated with oncogene amplification and poor outcome across multiple cancers. Nat Genet. 2020; 52:891-7.
31. Griffith M, Griffith O L. Mitelman Database (Chromosome Aberrations and Gene Fusions in Cancer). Dict Bioinforma Comput Biol. 2004; DOI: 10.1002/9780471650126.dob0996.
32. Flaherty K T, Infante J R, Daud A, Gonzalez R, Kefford R F, Sosman J, et al. Combined BRAF and MEK Inhibition in Melanoma with BRAF V600 Mutations. N Engl J Med. 2012; 367:1694-703.
33. Nazarian R, Shi H, Wang Q, Kong X, Koya R C, Lee H, et al. Melanomas acquire resistance to B-RAF(V600E) inhibition by RTK or N-RAS upregulation. Nature. 2010; 468:973-7.
34. Poulikakos P I, Persaud Y, Janakiraman M, Kong X, Ng C, Moriceau G, et al. RAF inhibitor resistance is mediated by dimerization of aberrantly spliced BRAF(V600E). Nature. 2011; 480:387-90.
35. Kemper K, Krijgsman O, Kong X, Cornelissen-Steijger P, Shahrabi A, Weeber F, et al. BRAFV600E Kinase Domain Duplication Identified in Therapy-Refractory Melanoma Patient-Derived Xenografts. Cell Rep. 2016; 16:263-77.
36. Paraiso KHT, Xiang Y, Rebecca V W, Abel E V., Chen Y A, Munko A C, et al. PTEN loss confers BRAF inhibitor resistance to melanoma cells through the suppression of BIM expression. Cancer Res. 2011; 71:2750-60.
37. Wagle N, Emery C, Berger M F, Davis M J, Sawyer A, Pochanard P, et al. Dissecting therapeutic resistance to RAF inhibition in melanoma by tumor genomic profiling. J Clin Oncol. 2011; 29:3085-96.
38. Sun C, Wang L, Huang S, Heynen GJJE, Prahallad A, Robert C, et al. Reversible and adaptive resistance to BRAF(V600E) inhibition in melanoma. Nature. 2014; 508:118-22.
39. Tsoi J, Robert L, Paraiso K, Galvan C, Sheu K M, Lay J, et al. Multi-stage Differentiation Defines Melanoma Subtypes with Differential Vulnerability to Drug-Induced Iron-Dependent Oxidative Stress. Cancer Cell. 2018; 33:890-904.e5.
40. Hugo W, Shi H, Sun L, Piva M, Song C, Kong X, et al. Non-genomic and Immune Evolution of Melanoma Acquiring MAPKi Resistance. Cell. 2015; 162:1271-85.
41. Moriceau G, Hugo W, Hong A, Shi H, Kong X, Yu C C, et al. Tunable-Combinatorial Mechanisms of Acquired Resistance Limit the Efficacy of BRAF/MEK Cotargeting but Result in Melanoma Drug Addiction. Cancer Cell. Elsevier Inc.; 2015; 27:240-56.
42. Xue Y, Martelotto L, Baslan T, Vides A, Solomon M, Mai T T, et al. An approach to suppress the evolution of resistance in BRAF V600E-mutant cancer. Nat Med. 2017; 23:929-37.
43. Kong X, Kuilman T, Shahrabi A, Boshuizen J, Kemper K, Song J Y, et al. Cancer drug addiction is relayed by an ERK2-dependent phenotype switch. Nature. Nature Publishing Group; 2017; 550:270-4.
44. L'Abbate A, Tolomeo D, Cifola I, Severgnini M, Turchiano A, Augello B, et al. MYC-containing amplicons in acute myeloid leukemia: genomic structures, evolution, and transcriptional consequences. Leukemia. 2018; 32:2152-66.
45. Luebeck J, Coruh C, Dehkordi S R, Lange J T, Turner K M, Deshpande V, et al. AmpliconReconstructor integrates NGS and optical mapping to resolve the complex structures of focal amplifications. Nat Commun. 2020; 11:4374.
46. Suda K, Tomizawa K, Osada H, Maehara Y, Yatabe Y, Sekido Y, et al. Conversion from the “oncogene addiction” to “drug addiction” by intensive inhibition of the EGFR and MET in lung cancer with activating EGFR mutation. Lung Cancer. 2012; 76:292-9.
47. Hong A, Moriceau G, Sun L, Lomeli S, Piva M, Damoiseaux R, et al. Exploiting drug addiction mechanisms to select against mapki-resistant melanoma. Cancer Discov. 2018; 8:74-93.
48. Alt F W, Kellems R E, Bertino J R, Schimke R T. Selective multiplication of dihydrofolate reductase genes in methotrexate-resistant variants of cultured murine cells. 1978. Biotechnology. 1992; 24:397-410.
49. Nunberg J H, Kaufman R J, Schimke R T, Urlaub G, Chasin L A. Amplified dihydrofolate reductase genes are localized to a homogeneously staining region of a single chromosome in a methotrexate-resistant Chinese hamster ovary cell line. Proc Natl Acad Sci USA. 1978; 75:5553-6.
50. Biedler J L, Melera P W, Spengler B A. Specifically altered metaphase chromosomes in antifolate-resistant Chinese hamster cells that overproduce dihydrofolate reductase. Cancer Genet Cytogenet. 1980; 2:47-60.
51. McVey M, Lee S E. MMEJ repair of double-strand breaks (director's cut): deleted sequences and alternative endings. Trends Genet. 2008; 24:529-38.
52. Hong A, Piva M, Liu S, Hugo W, Lomeli S H, Zoete V, et al. Durable suppression of acquired mek inhibitor resistance in cancer by sequestering mek from erk and promoting antitumor t-cell immunity. Cancer Discov. 2021; 11:714-35.
53. Klempner S J, Bordoni R, Gowen K, Kaplan H, Stephens P J, Ou SHI, et al.
Identification of BRAF kinase domain duplications across multiple tumor types and response to RAF inhibitor therapy. JAMA Oncol. 2016; 2:272-4.
54. Eleveld T F, Oldridge D A, Bernard V, Koster J, Daage L C, Diskin S J, et al. Relapsed neuroblastomas show frequent RAS-MAPK pathway mutations. Nat Genet. 2015; 47:864-71.
55. Bhang H E C, Ruddy D A, Radhakrishna V K, Caushi J X, Zhao R, Hims M M, et al.
Studying clonal dynamics in response to cancer therapy using high-complexity barcoding. Nat Med. 2015; 21:440-8.
56. Llabani E, Hicklin R W, Lee H Y, Motika S E, Crawford L A, Weerapana E, et al. Diverse compounds from pleuromutilin lead to a thioredoxin inhibitor and inducer of ferroptosis. Nat Chem. 2019; 11:521-32.
57. Haq R, Shoag J, Andreu-Perez P, Yokoyama S, Edelman H, Rowe G C, et al. Oncogenic BRAF regulates oxidative metabolism via PGC1α and MITF. Cancer Cell. 2013; 23:302-15.
58. Cesi G, Walbrecq G, Zimmer A, Kreis S, Haan C. ROS production induced by BRAF inhibitor treatment rewires metabolic processes affecting cell growth of melanoma cells. Mol Cancer. 2017; 16:102.
59. Trotta A P, Gelles J D, Serasinghe M N, Loi P, Arbiser J L, Chipuk J E. Disruption of mitochondrial electron transport chain function potentiates the pro-apoptotic effects of MAPK inhibition. J Biol Chem. 2017; 292:11727-39.
60. Schöckel L, Glasauer A, Basit F, Bitschar K, Truong H, Erdmann G, et al. Targeting mitochondrial complex I using BAY 87-2243 reduces melanoma tumor growth. Cancer Metab. 2015; 3:11.
61. Vazquez F, Lim J H, Chim H, Bhalla K, Girnun G, Pierce K, et al. PGC1α Expression Defines a Subset of Human Melanoma Tumors with Increased Mitochondrial Capacity and Resistance to Oxidative Stress. Cancer Cell. 2013; 23:287-301.
62. Shen S, Faouzi S, Souquere S, Roy S, Routier E, Libenciuc C, et al. Melanoma Persister Cells Are Tolerant to BRAF/MEK Inhibitors via ACOX1-Mediated Fatty Acid Oxidation. Cell Rep. 2020; 33:108421.
63. Del Rey M Q, Mancias J D. NCOA4-mediated ferritinophagy: A potential link to neurodegeneration. Front Neurosci. 2019; 13:238.
64. Gao M, Monian P, Pan Q, Zhang W, Xiang J, Jiang X. Ferroptosis is an autophagic cell death process. Cell Res. 2016; 26:1021-32.
65. Engelman J A, Zejnullahu K, Mitsudomi T, Song Y, Hyland C, Joon O P, et al. MET amplification leads to gefitinib resistance in lung cancer by activating ERBB3 signaling. Science. 2007; 316:1039-43.
66. Vandevanter D R, Piaskowski V D, Casper J T, Douglass E C, Von Hoff D D. Ability of circular extrachromosomal DNA molecules to carry amplified MYCN protooncogenes in human neuroblastomas in vivo. J Natl Cancer Inst. 1990; 82:1815-21.
67. Nathanson D A, Gini B, Mottahedeh J, Visnyei K, Koga T, Gomez G, et al. Targeted therapy resistance mediated by dynamic regulation of extrachromosomal mutant EGFR DNA. Science. 2014; 343:72-6.
68. Akbani R, Akdemir K C, Aksoy B A, Albert M, Ally A, Amin S B, et al. Genomic Classification of Cutaneous Melanoma. Cell. 2015; 161:1681-96.
69. Storlazzi C T, Lonoce A, Guastadisegni M C, Trombetta D, D'Addabbo P, Daniele G, et al. Gene amplification as doubleminutes or homogeneously staining regions in solid tumors: Origin and structure. Genome Res. 2010; 20:1198-206.
70. Decarvalho A C, Kim H, Poisson L M, Winn M E, Mueller C, Cherba D, et al. Discordant inheritance of chromosomal and extrachromosomal DNA elements contributes to dynamic disease evolution in glioblastoma. Nat Genet. Springer US; 2018; 50:708-17.
71. Oobatake Y, Shimizu N. Double-strand breakage in the extrachromosomal double minutes triggers their aggregation in the nucleus, micronucleation, and morphological transformation. Genes Chromosom Cancer. 2020; 59:133-43.
72. Gallant J N, Sheehan J H, Shaver T M, Bailey M, Lipson D, Chandramohan R, et al. EGFR kinase domain duplication (EGFR-KDD) is a novel oncogenic driver in lung cancer that is clinically responsive to afatinib. Cancer Discov. 2015; 5:1155-63.
73. Fenstermaker R A, Ciesielski M J, Castiglia G J. Tandem duplication of the epidermal growth factor receptor tyrosine kinase and calcium internalization domains in A-172 glioma cells. Oncogene. 1998; 16:3435-43.
74. Frederick L, Wang X Y, Eley G, James C D. Diversity and frequency of epidermal growth factor receptor mutations in human glioblastomas. Cancer Res. 2000; 60:1383-7.
75. Zhang J, Wu G, Miller C P, Tatevossian R G, Dalton J D, Tang B, et al. Whole-genome sequencing identifies genetic alterations in pediatric low-grade gliomas. Nat Genet. 2013; 45:602-12.
76. Corazao-Rozas P, Guerreschi P, André F, Gabert P E, Lancel S, Dekiouk S, et al. Mitochondrial oxidative phosphorylation controls cancer cell's life and death decisions upon exposure to MAPK inhibitors. Oncotarget. 2016; 7:39473-85.
77. Gopal Y N V, Rizos H, Chen G, Deng W, Frederick D T, Cooper Z A, et al. Inhibition of mTORC1/2 overcomes resistance to MAPK pathway inhibitors mediated by PGC1α and oxidative phosphorylation in melanoma. Cancer Res. 2014; 74:7037-47.
78. Schieber M, Chandel N S. ROS function in redox signaling and oxidative stress. Curr Biol. 2014; 24:R453-62.
79. Yuan L, Mishra R, Patel H, Alanazi S, Wei X, Ma Z, et al. Braf mutant melanoma adjusts to BRAF/MEK inhibitors via dependence on increased antioxidant SOD2 and increased reactive oxygen species levels. Cancers (Basel). 2020; 12:1-14.
80. Rahal A, Kumar A, Singh V, Yadav B, Tiwari R, Chakraborty S, et al. Oxidative stress, prooxidants, and antioxidants: The interplay. Biomed Res Int. 2014; 2014:761264.
81. Dixon S J, Lemberg K M, Lamprecht M R, Skouta R, Zaitsev E M, Gleason C E, et al. Ferroptosis: An iron-dependent form of nonapoptotic cell death. Cell. 2012; 149:1060-72.
82. Hou W, Xie Y, Song X, Sun X, Lotze M T, Zeh H J, et al. Autophagy promotes ferroptosis by degradation of ferritin. Autophagy. 2016; 12:1425-8.
83. Wang L, Leite de Oliveira R, Huijberts S, Bosdriesz E, Pencheva N, Brunen D, et al. An Acquired Vulnerability of Drug-Resistant Melanoma with Therapeutic Potential. Cell. 2018; 173:1413-1425.e14.
84. Snapka R M, Varshavsky A. Loss of unstably amplified dihydrofolate reductase genes from mouse cells is greatly accelerated by hydroxyurea. Proc Natl Acad Sci USA. 1983; 80:7533-7.
85. Yu L, Zhao Y, Quan C, Ji W, Zhu J, Huang Y, et al. Gemcitabine Eliminates Double Minute Chromosomes from Human Ovarian Cancer Cells. PLoS One. 2013; 8:e71988.
86. Von Hoff D D, McGill J R, Forseth B J, Davidson K K, Bradley T P, Van Devanter D R, et al. Elimination of extrachromosomally amplified MYC genes from human tumor cells reduces their tumorigenicity. Proc Natl Acad Sci USA. 1992; 89:8165-9.
87. Cai M, Zhang H, Hou L, Gao W, Song Y, Cui X, et al. Inhibiting homologous recombination decreases extrachromosomal amplification but has no effect on intrachromosomal amplification in methotrexate-resistant colon cancer cells. Int J Cancer. 2019; 144:1037-48.
88. Tian X, Liu C, Wang X, Wang F, Wang L, Xu L, et al. Sei-1 promotes double minute chromosomes formation through activation of the PI3K/Akt/BRCA1-Abraxas pathway and induces double-strand breaks in NIH-3T3 fibroblasts. Cell Death Dis. 2018; 9:341.
89. Sun W, Quan C, Huang Y, Ji W, Yu L, Li X, et al. Constitutive ERK1/2 activation contributes to production of double minute chromosomes in tumour cells. J Pathol. 2015; 235:14-24.
90. Watanabe T, Marotta M, Suzuki R, Diede S J, Tapscott S J, Niida A, et al. Impediment of Replication Forks by Long Non-coding RNA Provokes Chromosomal Rearrangements by Error-Prone Restart. Cell Rep. 2017; 21:2223-35.
91. Sondergaard J N, Nazarian R, Wang Q, Guo D, Hsueh T, Mok S, et al. Differential sensitivity of melanoma cell lines with BRAFV600Emutation to the specific Raf inhibitor PLX4032. J Transl Med. 2010; 8:39.
92. Kim D K. Estimating doubling time of cells in vitro. Vitr Cell Dev Biol—Anim J Soc Vitr Biol. 1995; 31:419-20.
93. Rajkumar U, Turner K, Luebeck J, Deshpande V, Chandraker M, Mischel P, et al. EcSeg: Semantic Segmentation of Metaphase Images Containing Extrachromosomal DNA. iScience. 2019; 21:428-35.
94. Robinson J T, Thorvaldsdóttir H, Winckler W, Guttman M, Lander E S, Getz G, et al. Integrative genomics viewer. Nat Biotechnol. 2011; 29:24-6.
95. Garvin T, Aboukhalil R, Kendall J, Baslan T, Atwal G S, Hicks J, et al. Interactive analysis and assessment of single-cell copy-number variations. Nat Methods. 2015; 12:1058-60.
96. Langmead B. Alignment with Bowtie. Curr Protoc Bioinforma. 2011; 1-24.
97. Li H. Aligning sequence reads, clone sequences and assembly contigs with BWA-MEM. arXiv Prepr arXiv. 2013;
98. Van der Auwera G A, O'Connor B. Genomics in the cloud: using Docker, GATK, and WDL in Terra. O'Reilly Media, Inc.; 2020.
99. Talevich E, Shain A H, Botton T, Bastian B C. CNVkit: Genome-Wide Copy Number Detection and Visualization from Targeted DNA Sequencing. PLoS Comput Biol. 2016; 12:e1004873.
100. Deshpande V, Luebeck J, Nguyen NPD, Bakhtiari M, Turner K M, Schwab R, et al. Exploring the landscape of focal amplifications in cancer using AmpliconArchitect. Nat Commun. 2019; 10:392.
101. Wala J A, Bandopadhayay P, Greenwald N F, O'Rourke R, Sharpe T, Stewart C, et al. SvABA: Genome-wide detection of structural variants and indels by local assembly. Genome Res. 2018; 28:581-91.
102. Raeisi Dehkordi S, Luebeck J, Bafna V. FaNDOM: Fast nested distance-based seeding of optical maps. Patterns. 2021; 2:100248.
103. Burgin J, Molitor C, Mohareb F. MapOptics: A light-weight, cross-platform visualization tool for optical mapping alignment. Bioinformatics. 2019; 35:2671-3.
104. Dobin A, Davis C A, Schlesinger F, Drenkow J, Zaleski C, Jha S, et al. STAR: Ultrafast universal RNA-seq aligner. Bioinformatics. 2013; 29:15-21.
105. Vivian J, Rao A A, Nothaft F A, Ketchum C, Armstrong J, Novak A, et al. Toil enables reproducible, open source, big biomedical data analyses. Nat Biotechnol. 2017; 35:314-6.
106. Van der Auwera G A, Carneiro M O, Hartl C, Poplin R, del Angel G, Levy-Moonshine A, et al. From fastQ data to high-confidence variant calls: The genome analysis toolkit best practices pipeline. Curr Protoc Bioinforma. 2013; 11.10.1-11.10.33.
107. Gu Z, Eils R, Schlesner M. Complex heatmaps reveal patterns and correlations in multidimensional genomic data. Bioinformatics. 2016; 32:2847-9.
108. Kemper K, Krijgsman O, Cornelissen-Steijger P, Shahrabi A, Weeber F, Song J, et al. Intra- and inter-tumor heterogeneity in a vemurafenib-resistant melanoma patient and derived xenografts. EMBO Mol Med. 2015; 7:1104-18.
109. Long G V., Fung C, Menzies A M, Pupo G M, Carlino M S, Hyman J, et al. Increased MAPK reactivation in early resistance to dabrafenib/trametinib combination therapy of BRAF-mutant metastatic melanoma. Nat Commun. 2014; 5:392.
110. Shen C H, Kim S H, Trousil S, Frederick D T, Piris A, Yuan P, et al. Loss of cohesin complex components STAG2 or STAG3 confers resistance to BRAF inhibition in melanoma. Nat Med. 2016; 22:1056-61.
111. Van Allen E M, Wagle N, Sucker A, Treacy D J, Johannessen C M, Goetz E M, et al. The genetic landscape of clinical resistance to RAF inhibition in metastatic melanoma. Cancer Discov. 2014; 4:94-109.
112. Villanueva J, Infante J R, Krepler C, Reyes-Uribe P, Samanta M, Chen H Y, et al. Concurrent MEK2 Mutation and BRAF Amplification Confer Resistance to BRAF and MEK Inhibitors in Melanoma. Cell Rep. 2013; 4:1090-9.
113. Wang V E, Xue J Y, Frederick D T, Cao Y, Lin E, Wilson C, et al. Adaptive Resistance to Dual BRAF/MEK Inhibition in BRAF-Driven Tumors through Autocrine FGFR Pathway Activation. Clin Cancer Res. 2019; 25:7202-17.
114. Wagle N, Van Allen E M, Treacy D J, Frederick D T, Cooper Z A, Taylor-Weiner A, et al. MAP kinase pathway alterations in BRAF-mutant melanoma patients with acquired resistance to combined RAF/MEK inhibition. Cancer Discov. 2014; 4:61-8.
115. Wang K, Li M, Hadley D, Liu R, Glessner J, Grant S F A, et al. PennCNV: An integrated hidden Markov model designed for high-resolution copy number variation detection in whole-genome SNP genotyping data. Genome Res. 2007; 17:1665-74.
116. Kuilman T, Velds A, Kemper K, Ranzani M, Bombardelli L, Hoogstraat M, et al. CopywriteR: DNA copy number detection from off-target sequence data. Genome Biol. 2015; 16:49.
117. Commo F, Guinney J, Ferté C, Bot B, Lefebvre C, Soria J C, et al. RCGH: A comprehensive array-based genomic profile platform for precision medicine. Bioinformatics. 2016; 32:1402-4.
118. Olshen A B, Venkatraman E S, Lucito R, Wigler M. Circular binary segmentation for the analysis of array-based DNA copy number data. Biostatistics. 2004; 5:557-72.
119. Ritz C, Baty F, Streibig J C, Gerhard D. Dose-response analysis using R. PLoS One. 2015; 10:e0146021.
120. Zamunér A R, Forti M, Andrade C P, Avila M A, da Silva E. Respiratory Sinus Arrhythmia and its Association with Pain in Women with Fibromyalgia Syndrome. Pain Pract. 2016; 16:704-11.
121. Müller J, Krijgsman O, Tsoi J, Robert L, Hugo W, Song C, et al. Low MITF/AXL ratio predicts early resistance to multiple targeted drugs in melanoma. Nat Commun. 2014; 5:5712.
122. Gerosa L, Chidley C, Frohlich F, Sanchez G, Lim S K, Muhlich J, et al. Receptor-Driven ERK Pulses Reconfigure MAPK Signaling and Enable Persistence of Drug-Adapted BRAF-Mutant Melanoma Cells. Cell Syst. 2020; 11:478-494.e9.
123. Ghandi M, Huang F W, Jand-Valbuena J, Kryukov G V., Lo C C, McDonald E R, et al. Next-generation characterization of the Cancer Cell Line Encyclopedia. Nature. 2019; 569:503-8.
124. Song C, Piva M, Sun L, Hong A, Moriceau G, Kong X, et al. Recurrent tumor cell-intrinsic and -extrinsic alterations during mapki-induced melanoma regression and early adaptation. Cancer Discov. 2017; 7:1248-65.
125. Li B, Dewey C N. RSEM: Accurate transcript quantification from RNA-Seq data with or without a reference genome. BMC Bioinformatics. 2011; 12:323.
126. Hanzelmann S, Castelo R, Guinney J. GSVA: Gene set variation analysis for microarray and RNA-Seq data. BMC Bioinformatics. 2013; 14:7.
127. Liberzon A, Birger C, Thorvaldsdóttir H, Ghandi M, Mesirov J P, Tamayo P. The Molecular Signatures Database Hallmark Gene Set Collection. Cell Syst. 2015; 1:417-25.
128. Subramanian A, Tamayo P, Mootha V K, Mukherjee S, Ebert B L, Gillette M A, et al. Gene set enrichment analysis: A knowledge-based approach for interpreting genome-wide expression profiles. Proc Natl Acad Sci USA. 2005; 102:15545-50.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method for treating melanoma in a subject comprising administering a composition comprising a ferroptosis-inducing agent to the subject; wherein the subject:
i) has amplified BRAF gene;
ii) has one or more of increased MITF expression, increased PGC-1α gene expression, increased mitochondrial respiration programs, increased expression of lipid oxidation pathways, insufficient upregulation of ROS detoxification pathways, decreased levels of reduced glutathione (GSH), reduced glutathione to oxidized glutathione (GSH/GSSG) ratios, decreased levels of total glutathione, and increased expression of NCOA4; or
iii) has been evaluated for peroxisome proliferator-activated receptor-γ coactivator (PGC-1α).

2. (canceled)

3. The method of claim 1, wherein the increased mitochondrial repiration programs comprises an increase in one or more of tricarboxylic acid (TCA) cycle, electron transport chain (ETC), oxidative phosphorylation, and mitochondrial biogenesis.

4. The method of claim 3, wherein increased expression of lipid oxidation pathways comprises increased expression or activity of PPARα and ACOX1 genes or proteins.

5. The method of claim 1, wherein insufficient upregulation of ROS detoxification pathways is determined by evaluating glutathione synthetase (GSS) and/or glutathione peroxidase 4 (GPX4).

6. (canceled)

7. The method of claim 1, wherein the method further comprises administration of an additional therapy and wherein the additional therapy comprises an immunotherapy.

8. (canceled)

9. The method of claim 7, wherein the immunotherapy comprises adoptive T cell transfer.

10. The method of claim 7, wherein the immunotherapy comprises a MAPK inhibitor.

11. The method of claim 10, wherein the MAPK inhibitor comprises a B-Raf inhibitor or a MEK inhibitor.

12-13. (canceled)

14. The method of claim 1, wherein the ferroptosis-inducing agent comprises a GPX4 inhibitor.

15. The method of claim 1, wherein the ferroptosis-inducing agent comprises one or more of erastin, sulfazine, and RSL3, salts or derivatives thereof.

16. The method of claim 1, wherein the subject has been determined to have:
amplified BRAF gene;
increased MITF expression; and/or
increased PGC-1α activity or expression.

17. The method of claim 1, wherein the subject has been previously treated for melanoma with a prior treatment and wherein the prior treatment comprises a MAPK inhibitor.

18. (canceled)

19. The method of claim 17, wherein the MAPK inhibitor comprises a B-Raf inhibitor or a MEK inhibitor.

20. The method of claim 19, wherein the B-Raf inhibitor comprises vemurafenib.

21-22. (canceled)

23. The method of claim 17, wherein the subject has been determined to be resistant to the prior treatment.

24. (canceled)

25. The method of claim 7, wherein the immunotherapy comprises an immune checkpoint inhibitor and wherein the immune checkpoint inhibitor comprises one or both of an anti-PD-1 antibody and an anti-CTLA4 antibody.

26. (canceled)

27. The method of claim 1, wherein the method further comprises determining one or more of BRAF gene amplification, MITF expression or activity, PGC-1α activity or expression, and combinations thereof, in a biological sample from the subject and wherein the biological sample comprises cancerous cells.

28. (canceled)

29. The method of claim 27, wherein MITF or PGC-1α is differentially expressed compared to a control and wherein the control comprises a non-cancerous sample, a MAPK inhibitor-sensitive cancerous sample, or an immunotherapy-resistant sample.

30-31. (canceled)

32. A method for classifying a subject diagnosed with melanoma, the method comprising:
a. obtaining a biological sample from the subject; and
b. detecting the expression or activity level of one or more biomarkers selected from MITF, PGC-1α, TCA cycle, ETC, oxidative phosphorylation, mitochondrial biogenesis, PPARα, ACOX1, GSS, GPX4; and/or the amplification of the BRAF gene in the biological sample from the subject.

33-51. (canceled)

52. A method of predicting sensitivity of melanoma cancer cells to ferroptosis inducers in a subject having melanoma, said method comprising:
a. obtaining a biological sample from the subject;
b. measuring one or more biomarkers comprising one or more of MITF, PGC-1α, TCA cycle, ETC, oxidative phosphorylation, mitochondrial biogenesis, PPARα, ACOX1, GSS, GPX4; and/or the amplification of the BRAF gene in the biological sample from the subject;
c. determining that the subject will be sensitive to ferroptosis inducing agents when MITF activity or expression is increased, PGC-1α activity or expression is increased, one or more of TCA cycle, ETC, oxidative phosphorylation, mitochondrial biogenesis are increased, PPARα and/or ACOX1 expression or activity is increased, GSS and/or GPX4 is insufficiently upregulated, and/or BRAF gene amplification is detected.

53-69. (canceled)


SEQUENCE LISTING (26 sequences)

SEQ ID NO: 1 - DNA, 20 bp, Artificial Sequence
  Synthetic Polynucleotide
tttagaacctcacgcacccc

SEQ ID NO: 2 - DNA, 20 bp, Artificial Sequence
  Synthetic Polynucleotide
tgttgtagttgtgagccgca

SEQ ID NO: 3 - DNA, 18 bp, Artificial Sequence
  Synthetic Polynucleotide
ctggcattgccctcaacg

SEQ ID NO: 4 - DNA, 25 bp, Artificial Sequence
  Synthetic Polynucleotide
agaagatgaaaagagttgtcagggc

SEQ ID NO: 5 - DNA, 98 bp, Artificial Sequence
  Synthetic Polynucleotide
ctgttaccttcagagggaactgttgggtgagttctggcacgtaggccctcactgctgtgtattgctacactgtaaatatactgcagtttatttggcca

SEQ ID NO: 6 - DNA, 92 bp, Artificial Sequence
  Synthetic Polynucleotide
accttcagagggaactgttgggtgagttctggcacgtaggccctcactgctgtgtattgctacactgtaaatatactgcagtttatttggcc

SEQ ID NO: 7 - DNA, 89 bp, Artificial Sequence
  Synthetic Polynucleotide
ctgttaccttcagagggaactgttgggtgagttctggcacgtaggccctcactgctgtgtattgctacactgtaaatatactgcagttt

SEQ ID NO: 8 - DNA, 45 bp, Artificial Sequence
  Synthetic Polynucleotide
gctgtgtattgctacactgtaaatatactgcagtttatttggcca

SEQ ID NO: 9 - DNA, 40 bp, Artificial Sequence
  Synthetic Polynucleotide
gtattgctacactgtaaatatactgcagtttatttggcca

SEQ ID NO: 10 - DNA, 22 bp, Artificial Sequence
  Synthetic Polynucleotide
ctgtaccttcagagggaactgt

SEQ ID NO: 11 - DNA, 22 bp, Artificial Sequence
  Synthetic Polynucleotide
ctgttaccttcagagggaactg

SEQ ID NO: 12 - DNA, 27 bp, Artificial Sequence
  Synthetic Polynucleotide
ctgttaccttcagagggaactgttggg

SEQ ID NO: 13 - DNA, 39 bp, Artificial Sequence
  Synthetic Polynucleotide
ctgttaccttcagagggaactgttgggtgagttctggca

SEQ ID NO: 14 - DNA, 62 bp, Artificial Sequence
  Synthetic Polynucleotide
ctgttaccttcagagggaactgttgggtgagttctggcacgtaggccctcactgctgtgtat

SEQ ID NO: 15 - DNA, 66 bp, Artificial Sequence
  Synthetic Polynucleotide
ctgttaccttcagagggaactgttgggtgagttctggcacgtaggccctcactgctgtgtattgct

SEQ ID NO: 16 - DNA, 72 bp, Artificial Sequence
  Synthetic Polynucleotide
ctgttaccttcagagggaactgttgggtgagttctggcacgtaggccctcactgctgtgtattgctacactg

SEQ ID NO: 17 - DNA, 73 bp, Artificial Sequence
  Synthetic Polynucleotide
ctgttaccttcagagggaactgttgggtgagttctggcacgtaggccctcactgctgtgtattgctacactgt

SEQ ID NO: 18 - DNA, 79 bp, Artificial Sequence
  Synthetic Polynucleotide
ctgttaccttcagagggaactgttgggtgagttctggcacgtaggccctcactgctgtgtattgctacactgtaaatat

SEQ ID NO: 19 - DNA, 96 bp, Artificial Sequence
  Synthetic Polynucleotide
gttaccttcagagggaactgttgggtgagttctggcacgtaggccctcactgctgtgtattgctacactgtaaatatactgcagtttatttggcca

SEQ ID NO: 20 - DNA, 81 bp, Artificial Sequence
  Synthetic Polynucleotide
ctgttacgggtgagttctggcacgtaggccctcactgctgtgtattgctacactgtaaatatactgcagtttatttggcca

SEQ ID NO: 21 - DNA, 93 bp, Artificial Sequence
  Synthetic Polynucleotide
ctgttaccttcagagggaactgttggttctggcacgtaggccctcactgctgtgtattgctacactgtaaatatactgcagtttatttggcca

SEQ ID NO: 22 - DNA, 89 bp, Artificial Sequence
  Synthetic Polynucleotide
ctgttaccttcagagggaactgttgggtgagttcggccctcactgctgtgtattgctacactgtaaatatactgcagtttatttggcca

SEQ ID NO: 23 - DNA, 60 bp, Artificial Sequence
  Synthetic Polynucleotide
ctgttaccttcagagggaactgttgggtgagttctggcacgtaggccctcactgctgtgt

SEQ ID NO: 24 - DNA, 70 bp, Artificial Sequence
  Synthetic Polynucleotide
ctgttaccttcagagggaactgttgggtgagttctggcacgtaggccctcactgctgtgtattgctacac

SEQ ID NO: 25 - DNA, 29 bp, Artificial Sequence
  Synthetic Polynucleotide
atcacttacacatcagaaagctggttata

SEQ ID NO: 26 - DNA, 15 bp, Artificial Sequence
  Synthetic Polynucleotide
agtagagatggggtc